(19) United States
(12) Patent Application Publication
(10) Pub. No.: US 2024/0083952 A1
(43) Pub. Date: Mar. 14, 2024

(54) ORDER AND DISORDER AS A DESIGN PRINCIPLE FOR STIMULI-RESPONSIVE BIOPOLYMER NETWORKS

(71) Applicant: Duke University, Durham, NC (US)

(72) Inventors: Ashutosh Chilkoti, Durham, NC (US); Stefan Roberts, Raleigh, NC (US)

(21) Appl. No.: 18/303,530

(22) Filed: Apr. 19, 2023

Related U.S. Application Data
(63) Continuation of application No. 16/625,899, filed on Dec. 23, 2019, now Pat. No. 11,680,083.
(60) Provisional application No. 62/527,836, filed on Jun. 30, 2017.
(60) Provisional application No. 62/534,019, filed on Jul. 18, 2017.

(51) Int. Cl. C07K 14/00; A61K 9/00; A61K 9/51; A61K 35/12; A61K 35/74

(52) U.S. Cl. CPC C07K 14/001; A61K 9/0024; A61K 9/5169; A61K 35/12; A61K 35/74

(57) ABSTRACT

Disclosed herein are partially ordered polypeptides, which include a plurality of disordered domains and a plurality of structured domains. The partially ordered polypeptides may have phase transition behavior and form aggregates at, above, or below certain temperatures. Further provided are cellular scaffolds comprised of the partially ordered polypeptides.

### CROSS-REFERENCE TO RELATED APPLICATIONS

This application is a continuation of U.S. patent application Ser. No. 16/625,899, filed Dec. 23, 2019, which is the U.S. national stage entry, under 35 U.S.C. 371, of International Application No. PCT/US2018/040409, filed Jun. 29, 2018, which claims priority to U.S. Provisional Patent Application No. 62/527,836, filed Jun. 30, 2017; and U.S. Provisional Patent Application No. 62/534,019, filed Jul. 18, 2017, the entire contents of each of which are hereby incorporated by reference.

### STATEMENT REGARDING FEDERALLY SPONSORED RESEARCH

This invention was made with government support under grant GM061232 awarded by the National Institutes of Health, and grant NSF DMR-11-21107 awarded by the National Science Foundation. The government has certain rights in the invention.

### SEQUENCE LISTING

The application includes a Sequence Listing which has been submitted electronically in XML format and is hereby incorporated by reference in its entirety. Said XML Sequence Listing, created on Aug. 9, 2023, is named 028193-9261-US04 Sequence Listing.xml, and is 29,441 bytes in size.

### FIELD

This disclosure relates to polypeptides with phase transition behavior and their use as cellular scaffolds.

### INTRODUCTION

Both purely crystalline and amorphous materials have been extensively studied for their interesting properties, but they comprise a very small portion of the total materials space. Most material properties are a consequence of the interplay between their ordered and disordered domains. This phenomenon is one of the hallmarks of biological materials. For example, silk fibers owe their extraordinary attributes to the interactions of ordered and disordered domains at the inter- and intra-molecular level. With the recent expansion of research on intrinsically disordered proteins (IDPs), the importance of disorder-order interactions has become further undeniable. To understand how this interplay creates macroscopic material properties, ordered and disordered nanoscale modules have to be synthesized with molecular precision. The emergence of genetically encoded synthesis of peptide polymers makes it possible to design building blocks with this level of control over sequence and structure. There is a need for advanced materials that can be rationally designed and precisely tuned.

### SUMMARY

In an aspect, the disclosure relates to a partially ordered polypeptide (POP) including: a plurality of disordered domains; and a plurality of structured domains, wherein the POP exhibits phase transition behavior.

In some embodiments, the disordered domain includes at least one of: (i) an amino acid sequence of [VPGXG]ₘ (SEQ ID NO:1), wherein X is any amino acid except proline and m is an integer greater than or equal to 1; (ii) a PG motif including an amino acid sequence selected from PG, P(X)ₙG (SEQ ID NO:2), and (B)ₘP(X)ₙG(Z)ₚ (SEQ ID NO:3), or a combination thereof, wherein m, n, and p are independently an integer from 1 to 15, and wherein U, X, and Z are independently any amino acid; (iii) a non-repetitive polypeptide including a sequence of at least 60 amino acids, wherein at least about 10% of the amino acids are proline (P), and wherein at least about 20% of the amino acids are glycine (G); (iv) a non-repetitive polypeptide including a sequence of at least 60 amino acids, wherein at least about 40% of the amino acids are selected from the group consisting of valine (V), alanine (A), leucine (L), lysine (K), threonine (T), isoleucine (I), tyrosine (Y), serine (S), and phenylalanine (F); and (v) a non-repetitive polypeptide including a sequence of at least 60 amino acids, wherein the sequence does not contain three contiguous identical amino acids, wherein any 5-10 amino acid subsequence does not occur more than once in the non-repetitive polypeptide, and wherein when the non-repetitive polypeptide includes a subsequence starting and ending with proline (P), the subsequence further includes at least one glycine (G). In some embodiments, the disordered domain includes an amino acid sequence of [VPGXG]ₘ (SEQ ID NO:1), wherein X is Val, or Ala, or mixture of Ala and Val, and wherein m is an integer from 1 to 50. In some embodiments, X is a mixture of Ala and Val in a ratio from 10:1 to 1:10 (Ala:Val). In some embodiments, X is a mixture of Ala and Val in a ratio of 1:1 or 1:4. In some embodiments, the structured domain includes at least one of: (i) a polyproline domain, each polyproline domain including at least 5 proline residues and having at least about 50% of the amino acids in a PPI polyproline helical conformation or a PPII polyproline helical conformation; and (ii) a polyalanine domain, each polyalanine domain including at least 5 alanine residues and having at least about 50% of the amino acids in an alpha-helical conformation. In some embodiments, the structured domain includes a polyalanine domain. In some embodiments, at least about 60% of the amino acids in each polyalanine domain are in an alpha-helical conformation. In some embodiments, the polyalanine domain includes an amino acid sequence of [Bₚ(A)_{q}Zᵣ]ₙ (SEQ ID NO:4) or [(BAₛ)ₜZᵣ]ₙ (SEQ ID NO:5), wherein B is Lys, Arg, Asp, or Glu; A is Ala; Z is Lys, Arg, Asp, or Glu; n is an integer from 1 to 50; p is an integer from 0 to 2; q is an integer from 1 to 50; r is an integer from 0 to 2; s is an integer from 1 to 5; and t is an integer from 1 to 50. In some embodiments, the structured domain includes (A)₂₅ (SEQ ID NO:6), K(A)₂₅K (SEQ ID NO:7), (KAAAA)₅K (SEQ ID NO:8), or D(A)₂₅K (SEQ ID NO:9), or a combination thereof. In some embodiments, the POP includes alternating disordered domains and structured domains. In some embodiments, about 4% to about 75% of the POP includes structured domains. In some embodiments, the POP is soluble below a lower critical solution temperature (LCST). In some embodiments, the POP has a transition temperature of heating (Tt-heating) and a transition temperature of cooling (Tt-cooling). In some embodiments, the transition temperature of heating (Tt-heating) and transition temperature of cooling (Tt-cooling) are identical. In some embodiments, the transition temperature of heating (Tt-heating) is greater than the transition temperature of cooling (Tt-cooling). In some embodiments, the transition temperature of heating (Tt-heating) is concentration-dependent. In some embodiments, the transition temperature of cooling (Tt-cooling) is concentration-independent. In some embodiments, the Tt-heating is primarily determined by the disordered domains, and wherein the Tt-cooling is primarily determined by the structured domains. In some embodiments, the POP forms an aggregate above the Tt-heating. In some embodiments, the aggregate resolubilizes when cooled to below the Tt-cooling. In some embodiments, the aggregate is a stable three-dimensional matrix. In some embodiments, the aggregate is fractal-like. In some embodiments, the aggregate is porous with a void volume. In some embodiments, the void volume is tunable.

In a further aspect, the disclosure relates to a scaffold including a plurality of the polypeptide as detailed herein at a temperature greater than the transition temperature, such that the polypeptide forms an aggregate. Another aspect of the disclosure provides a cellular scaffold including the scaffold as detailed herein and a plurality of cells.

In a further aspect, the disclosure relates to a method for forming a cellular scaffold, the method including: mixing cells with a plurality of the polypeptide as detailed herein at a first temperature less than the transition temperature of the polypeptide, such that the polypeptide does not form an aggregate; and incubating the polypeptides at a second temperature suitable for cellular growth and greater than the transition temperature, such that the polypeptides form an aggregate with the cells encapsulated within, to form the cellular scaffold. In some embodiments, the method further includes implanting the cellular scaffold into a subject.

In a further aspect, the disclosure relates to a method for forming a cellular scaffold, the method including: mixing cells with a plurality of the polypeptide as detailed herein to form a mixture, at a first temperature less than the transition temperature of the polypeptide, such that the polypeptide does not form an aggregate; and injecting the mixture at the first temperature into a subject, wherein the subject is at a second temperature greater than the transition temperature, such that the polypeptides form an aggregate with the cells encapsulated within, to form the cellular scaffold in the subject.

In a further aspect, the disclosure relates to a method for forming a scaffold, the method including: injecting into a subject a plurality of the polypeptide as detailed herein at a first temperature less than the transition temperature of the polypeptide, such that the polypeptide does not form an aggregate prior to injection, wherein the subject is at a second temperature greater than the transition temperature, such that the polypeptides form an aggregate to form the scaffold in the subject.

In some embodiments, the cells within the scaffold (e.g., injected along with the scaffold) integrate into the surrounding cells or tissues of the subject. In some embodiments, the cells of the subject surrounding the scaffold integrate into the scaffold. In some embodiments, the scaffold modifies the surrounding cells or tissues of the subject. In some embodiments, the cells within the scaffold, the cells integrating into the scaffold, or the cells modified by the scaffold form new vasculature. In some embodiments, the methods further include reducing the temperature to the first temperature, such that the aggregate/scaffold solubilizes; and separating the cells from the solubilized scaffold. In some embodiments, the separating step includes centrifugation. In some embodiments, the cells comprise stem cells, bacterial cells, or human tissue cells. In some embodiments, the scaffold has low immunogenicity or low antigenicity. In some embodiments, the scaffold promotes at least one of cell growth, recruitment, and differentiation.

In a further aspect, the disclosure relates to a drug delivery composition including: a plurality of POPs as detailed herein, self-assembled into an aggregate above the Tt-heating; and an agent encapsulated within the aggregate. In some embodiments, the drug delivery composition modifies the surrounding cells or tissues of the subject. In some embodiments, the drug delivery composition (and agent therein) recruits dendritic cells.

In a further aspect, the disclosure relates to a method of delivering an agent to a subject, the method including: encapsulating the agent in an aggregate, the aggregate including a plurality of POPs as detailed herein; and administering the aggregate to the subject.

In a further aspect, the disclosure relates to a method of treating a disease in a subject in need thereof, the method including administering the drug delivery composition as detailed herein to the subject. In some embodiments, administering the drug delivery composition results in the formation of new vasculature, wound healing, or a combination thereof in the subject.

In a further aspect, the disclosure relates to a method of increasing the maximum tolerated dose of an agent, the method including: encapsulating the agent in an aggregate of POPs as detailed herein; and administering the agent-encapsulated aggregate to a subject.

In some embodiments, the agent includes a small molecule, a polynucleotide, a polypeptide, a carbohydrate, or a combination thereof.

The disclosure provides for other aspects and embodiments that will be apparent in light of the following detailed description and accompanying figures.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1A-FIG. 1G: Partially ordered polymer library and structural characterization. (FIG. 1A) Recombinant POPs were constructed with 3 ELP components and 4 polyalanine helices at amino acid percentages up to 50%. Ultraviolet (UV)-Circular dichroism (CD) reveals definitive helical peaks at 222 and 208 nm, with peak amplitudes minimally altered by (FIG. 1B) polyalanine domain and (FIG. 1C) ELP but highly dependent on (FIG. 1D) total alanine content (dynode voltage >500 at <200 nm; data not used for analysis). (FIG. 1E) This structural signature is consistent with helix-coil predictions (Agadir). (FIG. 1F) ¹⁵N-HSQC and (FIG. 1G) H(N)CO (residue labels are the associated C′ of the previous residue) 2D solution NMR spectra for E1-H2-25% were used to more precisely quantify total structural content. Each polyalanine domain was determined to have an average helicity of 90%.

FIG. 2A-FIG. 2F: Turbidity and hysteresis. OD measurements as a function of temperature show sharp, reversible phase behavior and hysteresis for POPs. (FIG. 2A) Hysteresis scales as a function of total helical content. (FIG. 2B-FIG. 2D) For a given E(X), the composition of the alanine domain modulates the Tₜ-heating and Tₜ-cooling with greater hydrophilicity leading to increased temperatures. Hysteresis is also dependent on the composition (charge distribution) of the polyalanine domains with an increase in charge producing a decrease in hysteresis. The Tₜ-cooling is concentration independent and solely determined by the polyalanine domains. (FIG. 2E) Therefore, for a given H(X), the Tₜ-heating can be independently controlled with ELP composition, providing a method to orthogonally control Tₜ-heating and Tₜ-cooling. (FIG. 2F) Polymers can be cyclically heated and cooled with no change in thermal behavior. Optical density measurements were taken at 350 nm in PBS at 50 μM unless otherwise indicated. Heating and cooling rates were kept at 1° C./min. OD amplitudes are non-interpretable due to difference in aggregate formation and settling.

FIG. 3A-FIG. 3D: Proposed mechanism for hysteresis. Simulations of the hysteretic cycle were performed using a coarse-grained model. Heating and cooling were achieved by modulating the interaction strengths between ELP domains. (FIG. 3A) Snapshots extracted from a phenomenological simulation of POPs shown in the middle, surrounded by cartoon representations of the four states observed for POP during heating and cooling. Rod-like objects represent alanine domains and string-like tethers represent ELPs. The colors indicate their initial cluster with shading indicating different proteins in the same initial cluster. The one-sided arrows provide a pictorial summary of the expected rates for transitions between different states (fast for 2-3 and slow for 4-1). Within entangled aggregates we observe two types of morphologies viz., entangled spheres or entangled cylinders. There is a reversible spheres to cylinders transition at even higher temperatures. (FIG. 3B) A simplified representation of experimental data is annotated by the species populating each regime. The ordinate is labeled as a measure of optical density consistent with experimental work. (FIG. 3C-FIG. 3D) Enlarged snapshots from the cooling arm of panel (FIG. 3A) demonstrate that the highlighted POP is not able to isolate itself into a single cluster and that the decrease in aggregate density is limited by the presence of domain swapped proteins.

FIG. 4A-FIG. 4D: Arrested phase separation into fractal networks. (FIG. 4A) E1-H5-25% (2 mM, PBS) aggregation during a heating and cooling cycle shows a reversible transition from an optically translucent liquid to an opaque solid-like structure (passes inversion test) with syneresis observed at higher temperatures. (FIG. 4B) At the microscale, E1 and E1-H5-25% (400 μM, PBS) form liquid-like coacervates and fractal networks, respectively; scale bar 50 μm. (FIG. 4C) The intricacy of the network is more clearly seen with a 20 μm thick 3D reconstruction of E1-H5-25% (200 μM, PBS); scale bar 50 μM. (FIG. 4D) Network architecture at the meso scale is that of interconnected “beads on a string”, as revealed by SIM; scale bars 10 μm (left) and 1 μm (right).

FIG. 5A-FIG. 5C: Network stability and void volume. (FIG. 5A) As determined by the limited fluorescence recovery 25 min after bleaching, 12.5% and 25% networks have a high kinetic stability and limited liquid-like properties; Inset pictures are shown for E1-H5-25% at 400 μM. (FIG. 5B and FIG. 5C) Void volumes can be tuned from 60-90% by altering polymer concentration. Scale bars are 50 μm.

FIG. 6A-FIG. 6J: In vivo stability and tissue incorporation of POPs. (FIG. 6A) E1-H5-25% POP subcutaneous (s.c.) injections were significantly more stable than their E1 counterparts with just 5% of the injected dose degraded at 120 hrs; 200 μL 250 μM injections; p<0.05 for all data points after 0 hr. (FIG. 6B) Whereas ELPs diffuse into the s.c. space, POP depots were externally apparent, retaining the shape and volume of the initial injection up to dissection and ex vivo analysis. (FIG. 6C) Representative CT-SPECT images of the depots confirm increased diffusivity of ELPs and increased stability of POPs. (FIG. 6D) POPs were injected into BL/6 mice and explanted for analysis over 21 days. Representative images are shown with arrows pointing at externally evident vascularization of the biomaterial. (FIG. 6E) POPs rapidly integrated into the subcutaneous environment with sufficient strength to endure moderate extension less than 24 hours after injection. (FIG. 6F) There is a high initial cell incorporation with some change over the observed time periods; for *, p<0.05. (FIG. 6G and FIG. 6H) Flow cytometry for cells involved in the innate immune reveals subsequent spikes in neutrophils, inflammatory monocytes, and macrophages, with a loss in all hematopoietic cells (CD45+) by day 21; for *, p<0.05. (FIG. 6I) The loss in inflammation corresponds with an increase in vascularization, quantified by number of visible capillaries in histological sections (n=3 separate injections, with * p<0.05). (FIG. 6J) An example tissue slice 10 days post injection shows an area of particularly high vascularization density.

FIG. 7A-FIG. 7C: Purity of POPs. (FIG. 7A) All POPs, listed in (FIG. 7B), were purified to >95% as determined by SDS-PAGE gels. (FIG. 7C) The molecular weight (MW) of E1-H5-25% was confirmed by Matrix Assisted Laser Desorption/Ionization (MALDI) and is within 1.5% of the predicted MW. Small bands observed in the sodium dodecyl sulfate polyacrylamide gel electrophoresis (SDS-PAGE) are also confirmed to be truncation products of the longer polymer, likely the result of ribosomal pausing. The presence of small amounts of truncation product did not have discernable effects on the properties of POPs.

FIG. 8A-FIG. 8C: Additional structural characterization. (FIG. 8A) 12.5% polymers show indicative peaks at 208 and 222 nm, with all polyalanine domains showing similar peak amplitudes at 222 nm. (FIG. 8B) Peak amplitudes scale appropriately with the inclusion of helical domains for E1-H1 polymers. (FIG. 8C) E1-H3-25%, which does not transition at low temperatures, shows the preservation of helical signature peaks at high temperatures with some loss in peak amplitudes. (Data not used for analysis when dynode voltage >500 V at <200 nm for all).

FIG. 9A-FIG. 9C: Additional NMR analysis. The 15N-HSQC spectrum for E1-H1-25% (FIG. 9A) and E1-H2-25% (FIG. 9C) and the 13C-HSQC spectrum for E1-H2-25% (FIG. 9B) show the identifiable amino acid peaks despite the repetitiveness of the polymers. The addition salts in PBS does not appreciably alter the chemical shift positions for the polymers.

FIG. 10A-FIG. 10G: Additional turbidity data. (FIG. 10A) 12.5% POPs also show variability in hysteresis due to helix composition as well as (FIG. 10B) concentration independence and a tunable Tₜ-heating. (FIG. 10C) Tₜ-heating can be tuned by changing molecular weight without changing the total percentage of helicity, though Tₜ-cooling remains unaltered. (FIG. 10D) Attachment of Alexa Fluor 488 to the lysines on POPs does not significantly alter their phase behavior. Raw optical density measurements for (FIG. 10E) E1-H(X)-12.5% polymers, (FIG. 10F) E1-H(X)-25% polymers and (FIG. 10G) E1-H5-25% of different molecular weights illustrate their sharp phase behavior and varying degrees of hysteresis. Optical density measurements were taken in PBS. Heating and cooling rates were kept at 1° C./min. OD amplitudes are non-interpretable due to difference in aggregate formation, settling, and detector saturation.

FIG. 11A-FIG. 11D: Kinetics of hysteresis. Altering the (FIG. 11A) heating rates and (FIG. 11B) cooling rates also does not change the phase behavior, though some settling occurs at slower cooling rates. (FIG. 11C) Despite their hysteretic nature, polymers are capable of fully recovering from heating and cooling cycles. Ten cycles show no change in transition temperatures. (FIG. 11D) E1-H5-25% (50 μM, PBS) shows no recovery for 24 hours when heated and cooled to the hysteretic range above the Tₜ-cooling. Subsequent cooling after 24 hours shows rapid dissolution.

FIG. 12A and FIG. 12B: Temperature dependent CD. (FIG. 12A and FIG. 12B) E1-H1-25% shows a spectral shift consistent with distortions for helical peptides at the expected transition temperature. This polymer also shows an isodichroic point at 225 nm for both 12.5 and 25%. 10 μM, water, 1 mm path length for all experiments. Dynode voltage <500 for all.

FIG. 13A-FIG. 13E: Rheology. (FIG. 13A) A strain sweep from 0.01 to 100% reveals the linear viscoelastic region (LVER) of POPs. (FIG. 13B) Frequency sweeps within the LVER (1% strain) reveal solid-like material properties for POPs which scale non-linearly with concentration. (FIG. 13C) ELPs show more liquid-like behavior (G″>G′) and decrease mechanical integrity compared to POPs. (FIG. 13D) POPs exhibit plastic, frequency dependent viscosity whereas ELPs behave as Newtonian fluids. (FIG. 13E) Temperature sweeps of E1-H5-25% show an increase in elastic moduli as the polymer aggregates. The G′ plateau and drop-off after transition is likely due to aggregate shrinking and loss of contact with the plates. The change in normal force (F_{N}) (inset) reflects this loss of contact. All experiments in PBS after 30 min equilibration at 37° C.

FIG. 14A-FIG. 14C: Fractal Dimensions of POP Networks. (FIG. 14A) Single plane confocal images of E1-H5-25% in PBS were analyzed using a box counting algorithm in FracLac for ImageJ (FIG. 14B). The fractal dimension was determined graphically (FIG. 14C). Images for E1-H5-12.5% and 25% (n=3) revealed fractal dimensions ranging from 1.6 to 1.9 and varying with concentration.

FIG. 15A-FIG. 15C: Additional Structured Illumination Microscopy (SIM) images. The “beads-on-a-string” mesoscale architecture is consistent across concentrations and helical percentages; scale bar 10 μm. (FIG. 15A) E1-H5-25% at 100 μM; (FIG. 15B) E1-H5-12.5% at 100 μM; and (FIG. 15C) E1-H5-25% at 500 μM.

FIG. 16A-FIG. 16C: Biodistribution of POPs. (FIG. 16A) Body weight measures for all mice used are given along with the (FIG. 16B) dose of ¹²⁵I for each group. Dosages were used for data normalization and differences in doses on reflect an increase in bound iodine for POPs which is not expected to be experimentally relevant. (FIG. 16C) Radiation measured for organs after 120 hrs reveals some small distribution differences for POPs and ELPs, but none expected to be harmful.

FIG. 17A-FIG. 17F: Single photon emission computed tomography (SPECT)-Computed tomography (CT) analysis. (FIG. 17A) SPECT-CT images are shown for three mice imaged for SPECT analysis following s.c. injection of ELP in their right hind flank. (FIG. 17B) SPECT-CT images are shown for three mice imaged for SPECT analysis following s.c. injection of POP in their right hind flank. Analysis of ELP vs POP following injection, (FIG. 17C) volume; (FIG. 17D) surface to volume ratio; (FIG. 17E) surface area; and (FIG. 17F): activity density.

FIG. 18A-FIG. 18C: Excised depots. (FIG. 18A) Mice did not significantly change body weight across time points (day 1 and 3 not collected). POP (E1-H5-25%-120) and Matrigel depots are shown (FIG. 18B) pre- and (FIG. 18C) post-excision from the subcutaneous right flank. Scale bars=5 mm.

FIG. 19A-FIG. 19G: Flow cytometry gates for 250 μM POP. (FIG. 19A) Gates for removing cell debris and isolating singlets and live cells are shown. (FIG. 19B-FIG. 19G) Flow cytometry gating procedures to isolate all cell types are shown with examples from each time point for 250 μM POP samples. Cell subtypes are described in more detail in the Examples.

FIG. 20A-FIG. 20C: Flow gates for comparison groups. Additional example flow gates are shown for (FIG. 20A) Day 5 750 μM E1-H5-25%-120, (FIG. 20B) Day 5 Matrigel, and (FIG. 20C) Day 20 Matrigel.

FIG. 21A-FIG. 21E: Additional cell subtype analysis. (FIG. 21A) There was not significant change in the 250 μM POP (E1-H5-25%-120) subcutaneous injection sizes across all time points, though (FIG. 21B) total live cells did increase. (FIG. 21C) CD45+ subtypes for 250 μM POP show an initial spike in neutrophils, followed by monocytes and finally macrophages. (FIG. 21D) Epithelial cells and endothelial cells show little to no trend. (FIG. 21E) At day 5, the 250 μM and 750 μM POP groups were almost identical, with some slight change in the CD45+/− populations. * for p<0.05 for all plots. Isolated * notes significance to all other groups.

FIG. 22A-FIG. 22C: Additional Histological Analysis. (FIG. 22A) Histological slices for explanted E1-H5-25%-120 injections were collected from the center of the depots and stained with Hematoxylin and Eosin (H&E). (FIG. 22B) Representative slices for days 1-5 show cells present in the depots from day 1 with a visible increase in density over time. Cells primarily migrate from surrounding tissue (also note in FIG. 22C), but some denser internal regions can also be seen by day 5. (FIG. 22C) H&E stained slices from day 10 and day 21 show a continued increase in cell density and the emergence of vasculature. Colored dots (and arrows) on the slices indicate the presence of blood vessels or capillaries. At day 10, the vasculature is denser around the edges, near surrounding tissue, but it becomes more uniformly distributed by day 21. Of note, chronic inflammatory markers such as foreign body giant cells and the formation of thick fibrin capsules were not observed in any of the stained sections.

FIG. 23A-FIG. 23F: Comparison to Matrigel. (FIG. 23A) POPs recruit significantly more cells at day 5 and day 10. (FIG. 23B) An H&E stained Matrigel sample at Day 10 shows minimal cell recruitment and no vascularization. (FIG. 23C) POP shows significantly increase vascularization at day 10 (n=3). (FIG. 23D) POPs recruit more non-hematopoietic cells, (FIG. 23E) more neutrophils, fewer macrophages, and (FIG. 23F) more endothelial cells than Matrigel. * for p<0.05 for all plots.

FIG. 24A-FIG. 24G: Ultraviolet (UV) Crosslinkable POPs. (FIG. 24A) Through inclusion of the unnatural amino acid para-azidophenylalanine (pAzF), POPs can be made reactive in the presence of UV light. (FIG. 24B) The polymers tolerate the inclusion, retaining their thermal reversibility and hysteresis. (FIG. 24C) pAzF form nitrine groups under UV exposure capable of interacting non-specifically with protein chains. (FIG. 24D) As demonstrated by an SDS-page gel of the supernatant after reaction and centrifugation at 4° C., the reaction is rapid, with only 10 seconds of UV exposure required to fully remove all soluble components. (FIG. 24E) Fluorescent SDS-PAGE gel of POP made with and without the inclusion of pAzF in the production media. Az-POP contains pAzF residues that react with DBCO-Cy5, while the negative control is not fluorescently labeled via click chemistry. (FIG. 24F & FIG. 24G) Az-POP networks appear identical to POP networks without an Az component, and are not significantly different in void volume. Changes in concentration still allow tuning the void volume, as with non-Az-POPs.

FIG. 25A-FIG. 25G: Methods of altering mechanical stability. (FIG. 25A) Increasing polymer molecular weight or (FIG. 25B) the total polymer helicity increases mechanical stiffness. (FIG. 25C) Helix composition has a significant effect on G′, whereas the effect of (FIG. 25D) changing ELP composition is not as dramatic. (FIG. 25E) POP physical crosslinking can be combined with covalent, chemical crosslinking. The addition of a chemical crosslinker increases the elastic moduli. Using an ELP with equal and equivalently spaced lysines, we also show that chemical crosslinking increases ELP stability, though not to the same degree as POPs. (FIG. 25F) Matrigel also behaves as a solid, but soft gel. (FIG. 25G) The G′ at 1% strain and 1 Hz were compared to E1-H5-25%, and all polymers showed statistically (p<0.05) different mechanical properties except the change in ELP composition. All polymers were tested at 5 wt % in PBS with 30 min equilibrations at 37° C. prior to oscillations.

FIG. 26: Single Helix Polymer Coacervates. E1-H5-6.25% (200 μM, PBS), which contains only one helical domain per polymer, does not form a fractal network; rather, the polymer forms a colloidal suspension of coacervates, similar to that of ELP. Scale bars=20 μm, 10 μm for insert. Images from left to right show increase of heating.

FIG. 27A-FIG. 27C: Confocal microscopy analysis of POPs. Representative single z-slices, 20 μm z-stacks, and Imaris surface renders of z-stacks (including magnified insets) for E1-H5-25% at (FIG. 27A) 50 μM, (FIG. 27B) 200 μM, and (FIG. 27C) 800 μM. Scale bars=2 μm; scale bars in insets=10 μm.

FIG. 28A-FIG. 28B: Void volume and Composition. (FIG. 28A) Similar porosities were observed for all tested polymer compositions (200 μM, PBS), though the inclusion of a chemical cross-linker did have a slight, significant effect (p<0.05 compared to E1-H5-25% control.). (FIG. 28B) Representative z-stacks (20 μm thick) are shown for each tested composition. Scale bars=20 μm.

### DETAILED DESCRIPTION

Described herein are partially ordered polypeptides (POPs). The polypeptides have phase transition behavior, and may be used to form aggregates with a variety of uses such as scaffolds for cell growth. The polypeptides include a combination of structured domains and disordered domains. The ratio and length of the structured and disordered domains may be varied, which may be used to tune the temperature at which the polypeptides change phase(s).

Many natural biomaterials derive their meso and microscale properties from the nanoscale interactions of ordered domains and disordered regions. To mimic this multiscale synergy, we designed a set of partially ordered polypeptides (POPs) in which we precisely tune nanoscale order and disorder through design of amino acid sequences. The stimuli-responsiveness of the disordered components including an elastin-like polypeptide (ELP), and the structural stability of the ordered domains including polyalanine helices, combine to produce materials with emergent properties that are not realized in materials that include each sequence block alone. The resultant materials are thermally-responsive, fractal protein networks with tunable thermal stability. We have further explored the mechanisms driving these interactions using molecular dynamics simulations and have demonstrated their ability to support cell penetration and growth in vivo.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” as used herein as applied to one or more values of interest, refers to a value that is similar to a stated reference value. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Antigen” refers to a molecule capable of being bound by an antibody or a T cell receptor. The term “antigen”, as used herein, also encompasses T-cell epitopes. An antigen is additionally capable of being recognized by the immune system and/or being capable of inducing a humoral immune response and/or cellular immune response leading to the activation of B-lymphocytes and/or T-lymphocytes. In some embodiments, the antigen contains or is linked to a Th cell epitope. An antigen can have one or more epitopes (B-epitopes and T-epitopes). Antigens may include polypeptides, polynucleotides, carbohydrates, lipids, small molecules, and combinations thereof. Antigens may also be mixtures of several individual antigens. “Antigenicity” refers to the ability of an antigen to specifically bind to a T cell receptor or antibody and includes the reactivity of an antigen toward pre-existing antibodies in a subject. “Immunogenicity” refers to the ability of any antigen to induce an immune response and includes the intrinsic ability of an antigen to generate antibodies in a subject. As used herein, the terms “antigenicity” and “immunogenicity” are different and not interchangeable.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P.J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, TX; SAS Institute Inc., Cary, NC.). The healthy or normal levels or ranges for a target or for a protein activity may be defined in accordance with standard practice. A control may be an agent or cell without a POP. A control may be a molecule, or sample comprising a molecule, with a polypeptide or polymer, that is different from a POP as detailed herein, conjugated thereto or encapsulated within. A control may be a subject, or a sample therefrom, whose disease state is known. The subject, or sample therefrom, may be healthy, diseased, diseased prior to treatment, diseased during treatment, or diseased after treatment, or a combination thereof. The control may include, for example, an agent or cell alone or by itself.

The term “expression vector” indicates a plasmid, a virus or another medium, known in the art, into which a nucleic acid sequence for encoding a desired protein can be inserted or introduced.

The term “host cell” is a cell that is susceptible to transformation, transfection, transduction, conjugation, and the like with a nucleic acid construct or expression vector. Host cells can be derived from plants, bacteria, yeast, fungi, insects, animals, etc. In some embodiments, the host cell includes Escherichia coli.

“Polynucleotide” as used herein can be single stranded or double stranded, or can contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including uracil, adenine, thymine, cytosine, guanine, inosine, xanthine hypoxanthine, isocytosine, and isoguanine. Polynucleotides can be obtained by chemical synthesis methods or by recombinant methods.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three dimensional structure of a polypeptide monomer. “Quaternary structure” refers to the three dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Recombinant” when used with reference, e.g., to a cell, or nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed, or not expressed at all.

“Sample” or “test sample” as used herein can mean any sample in which the presence and/or level of a target is to be detected or determined or any sample comprising an agent, cell, or POP as described herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” as used herein can mean a mammal that wants or is in need of the herein described conjugates. The subject may be a patient. The subject may be a human or a non-human animal. The subject may be a mammal. The mammal may be a primate or a non-primate. The mammal can be a primate such as a human; a non-primate such as, for example, dog, cat, horse, cow, pig, mouse, rat, camel, llama, goat, rabbit, sheep, hamster, and guinea pig; or non-human primate such as, for example, monkey, chimpanzee, gorilla, orangutan, and gibbon. The subject may be of any age or stage of development, such as, for example, an adult, an adolescent, or an infant.

“Substantially identical” can mean that a first and second amino acid sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids.

“Treatment” or “treating,” when referring to protection of a subject from a disease, means preventing, suppressing, repressing, ameliorating, or completely eliminating the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

“Variant” as used herein with respect to a polynucleotide means (i) a portion or fragment of a referenced nucleotide sequence; (ii) the complement of a referenced nucleotide sequence or portion thereof; (iii) a polynucleotide that is substantially identical to a referenced polynucleotide or the complement thereof; or (iv) a polynucleotide that hybridizes under stringent conditions to the referenced polynucleotide, complement thereof, or a sequences substantially identical thereto.

A “variant” can further be defined as a peptide or polypeptide that differs in amino acid sequence by the insertion, deletion, or conservative substitution of amino acids, but retain at least one biological activity. Representative examples of “biological activity” include the ability to be bound by a specific antibody or polypeptide or to promote an immune response. Variant can mean a substantially identical sequence. Variant can mean a functional fragment thereof. Variant can also mean multiple copies of a polypeptide. The multiple copies can be in tandem or separated by a linker. Variant can also mean a polypeptide with an amino acid sequence that is substantially identical to a referenced polypeptide with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids. See Kyte et al., J. Mol. Biol. 1982, 157, 105-132. The hydropathic index of an amino acid is based on a consideration of its hydrophobicity and charge. It is known in the art that amino acids of similar hydropathic indexes can be substituted and still retain protein function. In one aspect, amino acids having hydropathic indices of ±2 are substituted. The hydrophobicity of amino acids can also be used to reveal substitutions that would result in polypeptides retaining biological function. A consideration of the hydrophilicity of amino acids in the context of a polypeptide permits calculation of the greatest local average hydrophilicity of that polypeptide, a useful measure that has been reported to correlate well with antigenicity and immunogenicity, as discussed in U.S. Pat. No. 4,554,101, which is fully incorporated herein by reference. Substitution of amino acids having similar hydrophilicity values can result in polypeptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant can be a polynucleotide sequence that is substantially identical over the full length of the full gene sequence or a fragment thereof. The polynucleotide sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

### 2. PARTIALLY ORDERED POLYPEPTIDE (POP)

Described herein are partially ordered polypeptides (POPs). Each POP may include a plurality of disordered domains, and a plurality of structured domains. The POP may exhibit phase transition behavior by changing solubility and aggregate dissolution/formation with temperature.

The disordered domains and the structured domains of the POP can be arranged in any number of possible ways. In some embodiments, one or more disordered domains are positioned between at least two adjacent structured domains of the POP. In some embodiments, the POP includes a plurality of structured domains repeated in tandem and a plurality of disordered domains repeated in tandem. In some embodiments, the plurality of structured domains repeated in tandem are positioned C-terminal to the plurality of disordered domains repeated in tandem. In some embodiments, the plurality of structured domains repeated in tandem are positioned N-terminal to the plurality of disordered domains repeated in tandem. In some embodiments, the POP is arranged as [disordered domain]_{q}-[structured domain]ᵣ-[disordered domain]ₛ-[structured domain]ₜ, wherein q, r, s, and t are independently an integer from 0 to 100, such as from 1 to 100, from 2 to 100, from 1 to 50 or from 2 to 50. In some embodiments, the POP is arranged as [disordered domain]_{q}-[structured domain]ᵣ, wherein q and r are independently an integer from 1 to 100. In some embodiments, q, r, s, and t are independently an integer from 0 to 10, from 0 to 20, from 0 to 30, from 0 to 40, from 0 to 50, from 0 to 60, from 0 to 70, from 0 to 80, from 0 to 90, from 0 to 100, from 1 to 10, from 1 to 20, from 1 to 30, from 1 to 40, from 1 to 150, from 1 to 60, from 1 to 70, from 1 to 80, from 1 to 90 or from 1 to 100.

a. Disordered Domain

The POP may include a plurality of disordered domains. The disordered domain may comprise any polypeptide that has minimal or no secondary structure as observed by CD, and have phase transition behavior. The disordered domain may include an amino acid sequence of repeated amino acids, non-repeated amino acids, or a combination thereof.

In some embodiments, about 20% to about 99%, such as about 25% to about 97%, about 35% to about 95% or about 50% to about 94% of the POP comprises disordered domains. At least about 2%, 3%, 4%, 5%, 10%, 15%, 20%, 25%, 30%, 35%, 40%, 45%, 50%, 55%, 60%, 65%, 70%, 75%, 80%, 85%, 90%, or 95% of the POP may comprise disordered domains.

In some embodiments, the disordered domain comprises an amino acid sequence of [VPGXG]ₘ (SEQ ID NO:1), wherein X is any amino acid and m is an integer greater than or equal to 1. In some embodiments, m is an integer from 1 to 500. In some embodiments, m is at least, at most, or exactly 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195,200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500. In some embodiments, m may be less than 500, less than 400, less than 300, less than 200, or less than 100. In some embodiments, m is from 1 to 500, from 1 to 400, from 1 to 300, from 1 to 200, or from 60 to 180. In some embodiments, m is 60, 120, or 180. In some embodiments, X is any amino acid except proline. In some embodiments, X is Val, or Ala, or a mixture of Ala and Val. In some embodiments, X is Val. In some embodiments, X is Ala. In some embodiments, X is a mixture of Ala and Val. In some embodiments, X is a mixture of Ala and Val in a ratio of 1:1, 1:2, 1:3, 1:4, 1:5, 1:6, 1:7, 1:8, 1:9, 1:10, 2:1, 3:1, 4:1, 5:1, 6:1, 7:1, 8:1, 9:1, or 10:1. In some embodiments, X is a mixture of Ala and Val in a ratio of 1:1 or 1:4. In some embodiments, X is a mixture of Ala and Val in a ratio from 10:1 to 1:10 (Ala:Val), such as from 5:1 to 1:5 or from 1:1 to 1:4.

In some embodiments, the disordered domain comprises a PG motif. The PG motif may include an amino acid sequence selected from PG, P(X)ₙG (SEQ ID NO:2), and (B)ₘP(X)ₙG(Z)ₚ (SEQ ID NO:3), or a combination thereof, wherein m, n, and p are independently an integer from 1 to 15, and wherein U, X, and Z are independently any amino acid. In some embodiments, m, n, and p are independently an integer less than 2, 3, 4, 5, 6, 7, 8, 9, 10, 11, 12, 13, 14, or 15. In some embodiments, m, n, and p are independently an integer greater than or equal to 1, 2, 3, 4, 5, 6, 7, 8, 9, 10, 11, 12, 13, 14, or 15. P(X)ₙG (SEQ ID NO:2) may include PXG, PXXG (SEQ ID NO:10), PXXXG (SEQ ID NO:11), PXXXXG (SEQ ID NO:12), PXXXXXG (SEQ ID NO:13), PXXXXXXG (SEQ ID NO:14), PXXXXXXXG (SEQ ID NO:15), PXXXXXXXXG (SEQ ID NO:16), PXXXXXXXXXG (SEQ ID NO:17), PXXXXXXXXXXG (SEQ ID NO:18), PXXXXXXXXXXXG (SEQ ID NO:19), PXXXXXXXXXXXXG (SEQ ID NO:20), PXXXXXXXXXXXXXG (SEQ ID NO:21), PXXXXXXXXXXXXXXG (SEQ ID NO:22), or PXXXXXXXXXXXXXXXG (SEQ ID NO:23), or a combination thereof. The disordered domain may further include additional amino acids at the C-terminal and/or N-terminal end of the PG motif. These amino acids surrounding the PG motif may also be part of the overall repeated motif. The amino acids that surround the PG motif may balance the overall hydrophobicity and/or charge so as to control the phase transition behavior of the disordered domain.

In some embodiments, the disordered domain comprises a non-repetitive polypeptide. The non-repetitive polypeptide may include a sequence of at least 60 amino acids, wherein at least about 10% of the amino acids are proline (P), and wherein at least about 20% of the amino acids are glycine (G). The non-repetitive polypeptide may include a sequence of at least 60 amino acids, wherein at least about 40% of the amino acids are selected from the group consisting of valine (V), alanine (A), leucine (L), lysine (K), threonine (T), isoleucine (I), tyrosine (Y), serine (S), and phenylalanine (F). The non-repetitive polypeptide may include a sequence of at least 60 amino acids, wherein the sequence does not contain three contiguous identical amino acids, wherein any 5-10 amino acid subsequence does not occur more than once in the non-repetitive polypeptide, and wherein when the non-repetitive polypeptide comprises a subsequence starting and ending with proline (P), the subsequence further comprises at least one glycine (G). The non-repetitive polypeptide may include less than about 50, 100, 200, 300, or 400 amino acids. The non-repetitive polypeptide may include at least about 50, 60, 70, 80, 90, or 100 amino acids.

b. Structured Domain

The POP may include a plurality of structured domains. The structured domain may have a secondary structure as observed by CD, such as, for example, an alpha helix. The structured domain may comprise at least one of a polyproline domain and a polyalanine domain. In some embodiments, the POP comprises alternating disordered domains and structured domains. In some embodiments, the structured domain comprises only polyalanine domains. In some embodiments, the structured domain comprises only polyproline domains.

In some embodiments, about 4% to about 75%, such as about 5% to about 70%, about 6% to about 60% or about 7% to about 50% of the POP comprises structured domains. At least about 2%, 3%, 4%, 5%, 10%, 15%, 20%, 25%, 30%, 35%, 40%, 45%, 50%, 55%, 60%, 65%, 70%, 75%, 80%, 85%, 90%, or 95% of the POP may comprise structured domains.

i) Polyalanine

In some embodiments, the structured domain comprises a polyalanine domain. Each polyalanine domain may include at least 5 alanine residues. Each polyalanine domain may have at least about 50% of the amino acids in an alpha-helical conformation. In some embodiments, the polyalanine domain comprises an amino acid sequence of [Bₚ(A)_{q}Zᵣ]ₙ (SEQ ID NO:4) or [(BAₛ)ₜZᵣ]ₙ (SEQ ID NO:5), or a combination thereof, wherein B is Lys, Arg, Asp, or Glu; A is Ala; Z is Lys, Arg, Asp, or Glu; n is an integer from 1 to 50; p is an integer from 0 to 2; q is an integer from 1 to 50; r is an integer from 0 to 2; s is an integer from 1 to 5; and t is an integer from 1 to 50. In some embodiments, the structured domain comprises (A)₂₅ (SEQ ID NO:6), K(A)₂₅K (SEQ ID NO:7), (KAAAA)₅K (SEQ ID NO:8), or D(A)₂₅K (SEQ ID NO:9), or a combination thereof.

ii) Polyproline

In some embodiments, the structured domain comprises a polyproline domain. Each polyproline domain may include at least 5 proline resides. Each polyproline domain may have at least about 50% of the amino acids in a Polyproline Helix I (PPI) polyproline helical conformation or a Polyproline Helix II (PPII) polyproline helical conformation, or a combination thereof. PPI includes a helical conformation with backbone dihedral angles of roughly [−75,160] and having cis isomers of the peptide bonds. PPII includes a helical conformation with backbone dihedral angles of roughly [−75,150] and having trans isomers of the peptide bonds. At least about 50%, 55%, 60%, 65%, 70%, 75%, 80%, 85%, 90%, or 95% of the amino acids in each polyproline domain may be in a helical conformation. In some embodiments, about 50% to about 95% of the amino acids in each polyproline domain are in helical conformation.

c. UV Crosslinkable Amino Acid Derivative

The POP may also include amino acid derivatives that are not naturally occurring, such as a UV crosslinkable amino acid derivative. The non-native amino acid derivative can be used to introduce covalent crosslinks between different POPs and within the same POP. For example, POPs that include the UV crosslinkable amino acid derivative can be exposed to UV light, which can result in covalent crosslinks being formed between the amino acid derivative and a side chain of an amino acid of another POP or with a side chain of an amino acid of the same POP (having the amino acid derivative). The UV crosslinkable amino acid derivative may be any amino acid that has been functionalized with an azide group. In some embodiments, the amino acid derivative is para-azidophenylalanine.

The UV crosslinkable amino acid derivative may be included at varying amounts without affecting the POP's ability to transition at different temperatures. For example, the UV crosslinkable amino acid derivative may be included within the POP from about 0.1% to about 20% (of the POP), such as from about 0.5% to about 15% or from about 1% to about 10% (of the POP).

d. Transition Behavior

The POP may demonstrate phase transition behavior by changing solubility and aggregate formation with temperature. The phase transition behavior of the POP may derive from the phase transition behavior of the disordered domains of the POP. “Phase transition” or “transition” may refer to the aggregation of a polypeptide, which occurs sharply at a specific temperature. The phase transition may be reversible, although the specific temperature of dissolution may be the same or different from the specific temperature of aggregation.

In some embodiments, the POP is soluble below a lower critical solution temperature (LCST). LCST is the temperature below which the polypeptide is miscible.

A transition temperature (Tt) is a temperature at which the POP changes from one state to another. States may include, for example, soluble polypeptides, gels, and aggregates of varying sizes and dimensions. The POP may have a transition temperature of heating (Tt-heating) and a transition temperature of cooling (Tt-cooling). In some embodiments, the transition temperature heating (Tt-heating) is concentration-dependent. In some embodiments, the transition temperature cooling (Tt-cooling) is concentration-independent. The Tt-heating may be primarily determined by the disordered domains. The Tt-cooling may be primarily determined by the structured domains.

Below the transition temperature (LCST or Tt), the POP may be highly soluble. Upon heating above the transition temperature, the POP may hydrophobically collapse and aggregate, forming a separate phase.

The POP may phase transition at a variety of temperatures. The POP may have a transition temperature from about 0° C. to about 100° C., from about 10° C. to about 50° C., or from about 20° C. to about 42° C. The transition temperature of heating (Tt-heating) and transition temperature of cooling (Tt-cooling) may be identical. As used herein, temperatures may be “identical” when the temperatures are within 2.0° C., 1.0° C., 0.5° C., or 0.1° C. of each other. In some embodiments, the transition temperature of heating (Tt-heating) is greater than the transition temperature of cooling (Tt-cooling). In embodiments where the POP has a Tt-heating greater than the Tt-cooling, the difference between the two transition temperatures may be referred to as a hysteresis. In some embodiments, the POP has a hysteresis of about 5° C. to about 70° C., such as about 5° C. to about 60° C. or about 10° C. to about 50° C.

The phase transition behavior of the POP may be utilized in purification of the POP according to a method referred to as “inverse transition cycling,” in which the POP's reversible phase transition behavior is used to cycle the solution through soluble and insoluble phases, thereby removing contaminants. Phase transition may also be triggered using kosmotropic salts, such as, for example, ammonium sulfate or sodium chloride. The kosmotropic salt may be added to a solution comprising the POP, with the kosmotropic salt being added until the POP forms aggregates or is precipitated out of solution. The aggregates may be pelleted by centrifugation and resuspended in a second solution or buffer. Aggregates of the POP may re-solubilize into solution once cooled below their Tt or when the kosmotropic salt is removed from the solution. In some embodiments, the POP is purified without any chromatographic purification. In some embodiments, the POP is generated recombinantly and purified from bacterial culture, such as, for example, from E. coli.

i) Aggregate

The POP may form an aggregate when the temperature is greater than the Tt-heating. The aggregate may resolubilize when cooled to below a temperature less than the Tt-cooling.

The aggregate formed by a plurality of POPs may have advantageous properties that can arise from the structure of the POPs. For example, the aggregate may have physical, non-covalent crosslinks. These physical, non-covalent crosslinks may arise from helical bundling of the structured domain(s) interacting with each other. The aggregate may also have covalent crosslinks (e.g., chemical crosslinks) in addition to physical, non-covalent crosslinks. Covalent crosslinks can be included in the aggregate in order to increase their mechanical stability without altering their porous architecture. In some embodiments, the aggregate can be formed from a plurality of POPs and can then be further stabilized by covalent crosslinking (after the formation of the aggregate). Covalent crosslinks can be introduced via a UV crosslinkable amino acid derivative having an azide functionality as described herein. Further examples of crosslinks that can be incorporated into the aggregate include, but are not limited to, small molecule crosslinks and cysteine disulfide bridges. An example of a chemical, small molecule crosslink is tetrakis(hydroxymethyl)phosphonium chloride (THPC), which can crosslink lysines within POPs.

In addition, the aggregate formed by a plurality of POPs may have solid-like properties that distinguish it from liquid-like coacervate structures. For example, the aggregate may have a storage modulus (G′) that is greater than its loss modulus (G″), such as having a G′ 2× greater, 5× greater, 10× greater, 15× greater, 20× greater, 25× greater, 30× greater, 35× greater, 50× greater or 100× greater than its G″. In some embodiments, the aggregate has a G′ from 2× greater to 100× greater than its G″, such as from 10× greater to 50× greater or from 20× greater to 35× greater than its G″.

The aggregate formed from a plurality of POPs may be a variety of sizes and dimensions. In some embodiments, the aggregate is a stable three-dimensional matrix. In some embodiments, the aggregate is fractal-like. In some embodiments, the aggregate is gel-like. In some embodiments, the aggregate is porous with a void volume, e.g., the non-protein rich phase of the aggregate. In some embodiments, the void volume is tunable. For example, the aggregate may have a void volume from about 60% to about 90% (of the volume of the aggregate). In addition, the aggregate may comprise pores having a diameter of about 1 μm to about 100 μm, such as about 1 μm to about 10 μm, about 3 μm to about 5 μm, about 25 μm to about 60 μm, about 30 μm to about 50 μm, or about 3 μm to about 50 μm.

### 3. POLYNUCLEOTIDES

Further provided are polynucleotides encoding the POPs detailed herein. A vector may include the polynucleotide encoding the POPs detailed herein. To obtain expression of a polypeptide, one may subclone the polynucleotide encoding the polypeptide into an expression vector that contains a promoter to direct transcription, a transcription/translation terminator, and if for a nucleic acid encoding a protein, a ribosome binding site for translational initiation. An example of a vector is pet24. Suitable bacterial promoters are well known in the art. Further provided is a host cell transformed or transfected with an expression vector comprising a polynucleotide encoding a POP as detailed herein. Bacterial expression systems for expressing the protein are available in, e.g., E. coli, Bacillus sp., and Salmonella (Paiva et al., Gene 1983, 22, 229-235; Mosbach et al., Nature 1983, 302, 543-545). Kits for such expression systems are commercially available. Eukaryotic expression systems for mammalian cells, yeast, and insect cells are well known in the art and are also commercially available. Retroviral expression systems can be used in the present invention.

The POP may be expressed recombinantly in a host cell according to one of skill in the art. The POP may be purified by any means known to one of skill in the art. For example, the POP may be purified using chromatography, such as liquid chromatography, size exclusion chromatography, or affinity chromatography, or a combination thereof. In some embodiments, the POP is purified without chromatography. In some embodiments, the POP is purified using inverse transition cycling.

### 4. SCAFFOLD

Further provided herein is a scaffold comprising a plurality of POPs. The scaffold may be formed at a temperature greater than the transition temperature of the POP, such that the polypeptide forms an aggregate. The scaffold may be injectable.

Further provided herein is a cellular scaffold. A cellular scaffold includes the scaffold and a plurality of cells. The cells may include a variety of types. In some embodiments, the cells comprise stem cells, bacterial cells, or human tissue cells, or a combination thereof.

The scaffold may have low immunogenicity or low antigenicity or both. The scaffold may promote at least one of cell growth, cell recruitment, and cell differentiation, or a combination thereof. The scaffold, or cellular scaffold, may be suitable for cell transplantation, tissue regeneration, cell culture, and cell-based in vitro assays. In addition, the scaffold and/or cellular scaffold may promote the formation of vasculature, wound healing, or a combination thereof.

### 5. DRUG DELIVERY COMPOSITION

Further provided herein is a drug delivery composition. The drug delivery composition may include a plurality of POPs as detailed herein, self-assembled into an aggregate above the Tt-heating, and an agent encapsulated within the aggregate.

a. Agent

The agent may be a therapeutic. In some embodiments, the agent is selected from a small molecule, nucleotide, polynucleotide, protein, polypeptide, carbohydrate, lipid, and a combination thereof. In some embodiments, the agent comprises a small molecule. In some embodiments, the agent comprises a protein. In some embodiments, the agent comprises a cancer therapeutic. In some embodiments, the agent recruits cells, such as, for example, dendritic cells. In some embodiments, the drug delivery composition recruits dendritic cells.

### 6. ADMINISTRATION

The POPs as detailed above can be formulated into a composition in accordance with standard techniques well known to those skilled in the pharmaceutical art. Accordingly, a composition may comprise the POP or aggregate thereof. The composition may be prepared for administration to a subject. Such compositions comprising a POP can be administered in dosages and by techniques well known to those skilled in the medical arts taking into consideration such factors as the age, sex, weight, and condition of the particular subject, and the route of administration.

The POP, aggregate thereof, or composition thereof can be administered prophylactically or therapeutically. In prophylactic administration, the POP can be administered in an amount sufficient to induce a response. In therapeutic applications, the POPs are administered to a subject in need thereof in an amount sufficient to elicit a therapeutic effect. An amount adequate to accomplish this is defined as “therapeutically effective dose.” Amounts effective for this use will depend on, e.g., the particular composition of the POP regimen administered, the manner of administration, the stage and severity of the disease, the general state of health of the patient, and the judgment of the prescribing physician. In some embodiments, the POP may be co-administered with an agent, cells, or a combination thereof.

The POP can be administered by methods well known in the art as described in Donnelly et al. (Ann. Rev. Immunol. 1997, 15, 617-648); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. The POP can be complexed to particles or beads that can be administered to an individual, for example, using a vaccine gun. One skilled in the art would know that the choice of a pharmaceutically acceptable carrier, including a physiologically acceptable compound, depends, for example, on the route of administration.

The POPs can be delivered via a variety of routes. Typical delivery routes include parenteral administration, e.g., intradermal, intramuscular or subcutaneous delivery. Other routes include oral administration, intranasal, intravaginal, transdermal, intravenous, intraarterial, intratumoral, intraperitoneal, and epidermal routes. In some embodiments, the POP is administered intravenously, intraarterially, or intraperitoneally to the subject.

### 7. METHODS

a. Methods for Forming a Cellular Scaffold

Provided herein are methods for forming a cellular scaffold. In some embodiments, the methods include mixing cells with a plurality of POPs at a first temperature less than the transition temperature of the polypeptide, such that the polypeptide does not form an aggregate, and incubating the POPs at a second temperature suitable for cellular growth and greater than the transition temperature, such that the polypeptides form an aggregate with the cells encapsulated within, to form the cellular scaffold. In some embodiments, the methods further include implanting the pre-formed cellular scaffold into a subject.

In some embodiments, the methods include mixing cells with a plurality of POPs to form a mixture, at a first temperature less than the transition temperature of the polypeptide, such that the polypeptide does not form an aggregate, and injecting the mixture at the first temperature into a subject, wherein the subject is at a second temperature greater than the transition temperature, such that the polypeptides form an aggregate with the cells encapsulated within, to form the cellular scaffold in the subject.

In some embodiments, the methods include injecting into a subject a plurality of POPs at a first temperature less than the transition temperature of the polypeptide, such that the polypeptide does not form an aggregate prior to injection, wherein the subject is at a second temperature greater than the transition temperature, such that the polypeptides form an aggregate to form the scaffold in the subject.

In some embodiments, the methods further include reducing the temperature to the first temperature, such that the aggregate/scaffold solubilizes, and separating the cells from the solubilized scaffold. The first temperature may be the Tₜ-cooling. In some embodiments, the separating step comprises centrifugation.

In some embodiments, the cells within the scaffold integrate into the surrounding cells or tissues of the subject. In some embodiments, the cells of the subject surrounding the scaffold integrate into the scaffold. In some embodiments, the cells within the scaffold modify the surrounding cells or tissues of the subject.

In some embodiments, the cells within the scaffold, the cells integrating into the scaffold, or the cells modified by the scaffold form new vasculature. The new vasculature may be formed within about 10 days of administration. In some embodiments, the newly created vasculature comprises capillaries or vasculature having capillary-like characteristics. In some embodiments, the newly created vasculature comprises arterioles or vasculature having arteriole-like characteristics. In some embodiments, the newly created vasculature comprises capillaries, arterioles or a combination thereof.

b. Methods of Delivering an Agent to a Subject

Provided herein are methods of delivering an agent to a subject. The methods may include encapsulating the agent in an aggregate, the aggregate comprising a plurality of POPs as detailed herein, and administering the aggregate to the subject.

c. Methods of Treating a Disease in a Subject

Provided herein are methods of treating a disease in a subject. The methods may include administering a drug delivery composition, scaffold, and/or cellular scaffold as detailed herein to the subject.

In some embodiments, the methods may include treating a disease where new vascularization is needed at a site of injury (e.g., vascular tissue engineering). The methods may include administering a drug delivery composition, scaffold, and/or cellular scaffold as described herein to a site of injury, e.g., a site of injury that needs creation of new vasculature. The methods of creating new vasculature for treating a disease may form the new vasculature within about 10 days of administration of the drug delivery composition, scaffold, and/or cellular scaffold. In some embodiments, the newly created vasculature comprises capillaries or vasculature having capillary-like characteristics. In some embodiments, the newly created vasculature comprises arterioles or vasculature having arteriole-like characteristics. In some embodiments, the newly created vasculature comprises capillaries, arterioles or a combination thereof. Further description on vascular tissue engineering can be found in Serbo et al., Stem Cell Res. Ther., 2013 Jan. 24; 4(1):8 and Lovett et al., Tissue Eng. Part B Rev., 2009 Sep.; 15(3):353-70, both of which are incorporated by reference herein in their entirety.

In some embodiments, the methods may include treating a disease where the administration of the drug delivery composition, scaffold, and/or cellular scaffold disclosed herein results in wound healing. For example, the methods may include administering a drug delivery composition, scaffold, and/or cellular scaffold as described herein to a site of injury, e.g., a site of injury in need of wound healing. The site of injury can be an acute injury or a chronic injury. An example of an acute injury includes, but is not limited to, a deep cut. Examples of a chronic injury include, but are not limited to, diabetic foot ulcers, pressure ulcers, and venous leg ulcers. In some embodiments, the disease in need of wound healing is diabetes mellitus and the site of injury in need of wound healing is a diabetic ulcer. Further description on wound healing can be found in W. International. Acellular Matrices for the Treatment of Wounds (2011); W. International. Best Practice Guidelines: Wound Management in Diabetic Foot Ulcers (2013); and Jarbrink, K. et al. Prevalence and incidence of chronic wounds and related complications: a protocol for a systematic review. Syst Rev 5, 152; all of which are incorporated by reference herein in their entirety.

d. Methods of Increasing the Maximum Tolerated Dose of an Agent

Provided herein are methods of increasing the maximum tolerated dose of an agent. The methods may include encapsulating the agent in an aggregate of POPs as detailed herein, and administering the agent-encapsulated aggregate to a subject.

### 8. EXAMPLES

### Example 1

### Materials and Methods

Synthesis of polymer genes. All polymers were cloned into a modified pet24 vector using a previously described process known as recursive directional ligation by plasmid reconstruction (PRe-RDL)(McDaniel, J. R., et al. Biomacromolecules 2010, 11, 944-952). Briefly, single stranded oligomers encoding the desired sequences were annealed into cassettes with CC and GG overhangs. The overhangs enabled their concatemerization and ligation (Quick Ligase, NEB, Ipswich, MA) into the pet24 vector. Using this process, we created a library of elastin-like polypeptide and polyalanine cassettes which could be pieced together through multiple cycles of PRe-RDL to form the final partially ordered polymers. All of the base oligomer cassettes used for polymer construction can be found below. Plasmids were transfected into chemically competent Eb5a (EdgeBio, Gaithersburg, MD) cells for cloning and BL21(DE3) (EdgeBio, Gaithersburg, MD) cells for protein expression. Sequences are shown in TABLE 4.

TABLE 4
DNA Cassettes for Pre-RDL.

| | | |
|---|---|---|
| E1 | Forward | TGTGGGTGTTCCGGGCGTAGGTGTCCCAGGTGTGGGCGTACC |
| | | GGGCGTTGGTGTTCCTGGTGTCGGCGTGCCGGG (SEQ ID |
| | | NO: 24) |
| | Reverse | CGGCACGCCGACACCAGGAACACCAACGCCCGGTACGCCCAC |
| | | ACCTGGGACACCTACGCCCGGAACACCCACACC (SEQ ID |
| | | NO: 25) |
| E2 | Forward | CGTGGGTGTTCCGGGCGTAGGTGTCCCAGGTGCGGGCGTACC |
| | | GGGCGTTGGTGTTCCTGGTGTCGGCGTGCCGGG (SEQ ID |
| | | NO: 26) |
| | Reverse | CGGCACGCCGACACCAGGAACACCAACGCCCGGTACGCCCG |
| | | CACCTGGGACACCTACGCCCGGAACACCCACGCC (SEQ ID |
| | | NO: 27) |
| E3 | Forward | CGCCGGAGTGCCAGGCGTGGGTGTTCCAGGAGCAGGCGTTC |
| | | CAGGTGTGGGTGTTCCTGG (SEQ ID NO: 28) |
| | Reverse | AGGAACACCCACACCTGGAACGCCTGCTCCTGGAACACCCAC |
| | | GCCTGGCACTCCGGCGCC (SEQ ID NO: 29) |
| H1 | Forward | TGCGGCCGCAGCTGCGGCGGCAGCCGCGGCTGCCGCGGCTG |
| | | CAGCGGCAGCCGCGGCTGCGGCGGCCGCAGCTGCGGG (SEQ |
| | | ID NO: 30) |
| | Reverse | CGCAGCTGCGGCCGCCGCAGCCGCGGCTGCCGCTGCAGCCG |
| | | CGGCAGCCGCGGCTGCCGCCGCAGCTGCGGCCGCACC (SEQ |
| | | ID NO: 31) |
| H2 | Forward | TAAAGCGGCCGCAGCTGCGGCGGCAGCCGCGGCTGCCGCGG |
| | | CTGCAGCGGCAGCCGCGGCTGCGGCGGCCGCAGCTGCGAAA |
| | | GG (SEQ ID NO: 32) |
| | Reverse | TTTCGCAGCTGCGGCCGCCGCAGCCGCGGCTGCCGCTGCAG |
| | | CCGCGGCAGCCGCGGCTGCCGCCGCAGCTGCGGCCGCTTTA |
| | | CC (SEQ ID NO: 33) |
| H3 | Forward | TAAAGCGGCCGCAGCTAAAGCCGCGGCAGCGAAAGCAGCCGC |
| | | GGCGAAAGCCGCAGCTGCGAAAGCGGCAGCCGCGAAGGG |
| | | (SEQ ID NO: 34) |
| | Reverse | CTTCGCGGCTGCCGCTTTCGCAGCTGCGGCTTTCGCCGCGGC |
| | | TGCTTTCGCTGCCGCGGCTTTAGCTGCGGCCGCTTTACC |
| | | (SEQ ID NO: 35) |
| H5 | Forward | TGATGCGGCCGCAGCTGCGGCGGCAGCCGCGGCTGCCGCGG |
| | | CTGCAGCGGCAGCCGCGGCTGCGGCGGCCGCAGCTGCGAAA |
| | | GG (SEQ ID NO: 36) |
| | Reverse | TTTCGCAGCTGCGGCCGCCGCAGCCGCGGCTGCCGCTGCAG |
| | | CCGCGGCAGCCGCGGCTGCCGCCGCAGCTGCGGCCGCATCA |
| | | CC (SEQ ID NO: 37) |

Expression and purification of POPs. For protein expression, 5 mL starter cultures were grown overnight from −80° C. DMSO stocks. Cells were then pelleted, resuspended in 1 mL of terrific broth, and used, along with 1 mL 100 μg mL⁻¹ of kanamycin (EMD Millipore, Billerica, MA) to inoculate 1 L of media. Cells were shaken at 200 rpm for 8 hrs at 25° C. before induction. For induction of protein expression, 1 mL of 1 M isopropyl β-D-1-thiogalactopyranoside (Goldbio, St. Louis, MO) was added to the flask and cultures were placed at 16° C. and 200 rpm overnight. Expression at lower temperature was necessary to prevent the formation of truncation products at ELP-polyalanine junctions. Cells were then pelleted and resuspended in 10 mL of 1×PBS for every 1 L of culture grown. Pulse sonication on ice, with a total active time of 3 minutes, was used to lyse cells. Cell lysates were treated with 10% PEI (MP Biomedical, Santa Ana, CA) (2 mL L⁻¹ culture) to remove contaminating DNA and centrifuged at 14k rpm for 10 min at 4° C. Polymer was purified from the resulting soluble fraction using a modified version of inverse thermal cycling (Meyer, D. E. et al. Nat. Biotechnol. 1999, 17, 1112-1115). The fraction was heated to 65° C. or until a phase separation was observed. For more hydrophilic polymers, this often required the addition of 1-2 M NaCl to depress the transition temperature. Once aggregated, the polymer solutions were centrifuged at 14k rpm for 10 min at 35° C., and the resulting pellet was re-suspended in 5-10 mL PBS. The heating and cooling centrifugation cycles were repeated 2-3 more times until a purity of 95% was achieved, as analyzed by SDS-PAGE. Pure polymers were dialyzed at 4° C. with frequent water changes for 2 days and lyophilized for storage.

Secondary structure characterization. Circular dichroism experiments were carried using an Aviv Model 202 instrument and 1 mm quartz cells (Hellma USA, Plainview, NY). Unless otherwise noted, scans were carried out in PBS (pH=7.4) with a polymer concentration of 10 μM. Polymers were scanned in triplicate from 260 nm to 185 nm in 1 nm steps with a 1 s averaging time. Data points with a dynode voltage above 500V were ignored for analysis. All measurements were done at 20° C. unless otherwise specified. Temperature ramping was done in 5° C./min increments with a 1 min equilibration at each step.

For NMR, polymers were grown in M9 minimal media with ¹⁵N—NH₄Cl and ¹³C-Glucose (Cambridge Isotopes, Tewksbury, MA) as the only nitrogen and carbon sources to ensure protein labelling. Samples were prepared in PBS (pH=7.4) unless otherwise noted. All NMR spectra were collected on an INOVA 600 (Varian Instruments, Palo Alto, CA) spectrometer with a triple resonance cryoprobe equipped with a z-field gradient coil. Resonance assignments were made using a set of triple resonance experiments including HNCO, HN(CA)CO, HN(CO)CA, HNCA, HCAN, and HCA(CO)N. The NMR spectra were processed using NMRpipe (Delaglio, F. et al. Journal of Biomolecular NMR 1995, 6), and were analyzed using NMRviewJ. Chemical shifts in the proton dimension were referenced relative to TMSP (trimethylsilylpropanoic acid) as 0 ppm. Quantification of helicity was accomplished using the identified alanine peaks of the H(N)CO spectra for E1-H2-25%. Chemical shift positions were placed on a spectrum of values ranging from fully disordered (177.19 ppm) to fully helical (180.78 ppm), as determined Vendruscolo et al. (De Simone, A., et al. J. Am. Chem. Soc. 2009, 131, 16332-16333; Camilloni, C., et al. Biochemistry 2012, 51, 2224-2231) and the central alanine peak of the 15° C. H(N)CO respectively, producing the values in TABLE 2. The method to calculate helicity was adapted from 62D algorithm developed by Vendruscolo et al. (Camilloni, C., et al. Biochemistry 2012, 51, 2224-2231). Alanines corresponding to carbon chemical shifts of peaks 2-7 were designated as fringe amino acids at the edges of the helix. This designation is consistent with our helix-coil transition theory prediction in which 6 alanines occur at values lower than the core set. All other alanines were assumed to be in the helix core. A subsequent averaging of the helicity values produces a helicity for each H2 polyalanine domain of 91%.

TABLE 2
CD Structural Analysis.

| | Helix | Beta Sheet | Turn | Disorder |
|---|---|---|---|---|
| Polymers | (%) | (%) | (%) | (%) |
| (ELP1)₈₀ | 1.6 | 23.9 | 14.8 | 59.7 |
| ELP1-H1-25% | 47 | 13.7 | 6.1 | 33.2 |
| ELP1-H2-25% | 35.8 | 34.5 | 0.3 | 29.4 |
| ELP1-H3-25% | 27.5 | 44.9 | 0 | 27.6 |
| ELP1-H4-25% | 40.1 | 16.8 | 6.5 | 36.6 |
| ELP1-H5-12.5% | 19.4 | 32.1 | 4.4 | 44.1 |

Temperature-dependent turbidity. The transition temperature (Tt) of each sample was determined by monitoring the optical density at 350 nm as a function of temperature on a UV-vis spectrophotometer (Cary 300 Bio; Varian Instruments, Palo Alto, CA) equipped with a multicell thermoelectric temperature controller. The Tt was defined as the point of greatest inflection (maximum of the first derivative) for the optical density. Unless otherwise stated, all samples were heated and cooled at 1° C. min⁻¹ in PBS at concentrations between 10 and 1000 μm.

Molecular dynamics simulations. The phenomenological simulations were designed to test the role of having two energy scales on the coarse structural features. We chose the interaction strengths of the ELP beads such that this range would span from highly soluble to aggregating polymers. This was quantified by running simulations with a range of energies and after equilibrating for 100 ns, decreasing these interaction strengths by 0.05 kcal/mol every 25 ns. We then quantified the number of polymers in the largest cluster, where two proteins were considered interacting if two beads were within 8 Angstroms, as a proxy for aggregation versus solubility in our simulations. These polymers were strongly aggregating with an interaction strength of 0.35 kcal/mol and readily disaggregated when that interaction dropped to 0.25 kcal/mol. As such, we used a range of interactions strengths for the ELPs that spanned at least 0.05 kcal/mol to 0.40 kcal/mol. This range of interaction strengths is our simulation equivalent to increasing the temperature of the system from below the LCST to above the LCST. Unfortunately, without any further constraints, we cannot be more quantitative in the scaling between the strength of our interactions and the experimental equivalent temperatures. We used a similar technique to parameterize the alanine domain bead interaction strength. Here our constraint in choosing an interaction strength is based on being strong enough to push it significantly into the aggregation prone regime. As such, we used interaction strengths of at least 1 kcal/mol for the alanine beads.

To test for effects related to hysteresis we utilized two different schemes for initial conditions. The first scheme, denoted the dimer initial conditions, was designed to create states that we think are representative of the pathway that the system will pass through as it approaches the LCST from below. Simulations of two proteins were equilibrated for 100 ns in a simulation box of 250A. This allowed the alanine domains in these dimers to pre-aggregate into a core. 25 different conformations of these dimers were then randomly placed in the simulations for the full system. At high ELP interaction strengths these simulations docked together. This means that the ELPs that are exposed around the alanine cores find each other. There is some degree of alanine cores merging together into larger cores that converge toward their thermodynamically favorable radius.

The second scheme, denoted the coil initial conditions, was designed to create a thermodynamically equilibrated aggregated state that we think the dimer initial condition simulations would eventually converge toward. We started the simulation with each polymer generated randomly. The only correction was to prevent steric clashes. These simulations showed a rapid initial collapse as the alanine domains found other alanine domains, and, if the ELP domains were above the LCST, the collapse of ELP domains as well. These simulations converged toward conformations with clusters of alanine domains that were well connected. After equilibrating for 100 ns, the interaction strength of the ELPs were decreased by 0.10 kcal/mol every 25 ns to model crossing from above the LCST to below the LCST. These simulations showed swelling as the ELPs no longer favored being in a high density but the connectivity of alanine domains between the two domains prevented the system from separating.

Fluorescence imaging and analysis. POPs were fluorescently labeled using Alexa Fluor 488 NHS Ester (Thermo Fisher, Waltham, MA) with a reaction efficiency of 20%. Excess dye was removed with dialysis and polymers were lyophilized for storage. For all experiments, the dyed polymers were diluted into an undyed stock such that no more than 5% of POPs in solution were labelled. Confocal images were taken on a Zeiss 710 inverted microscope with temperature controlled incubation. To prevent dehydration, 50 μL of sample solution was added to 384 well #1.5 glass bottom plates (Cellvis, Mountain View, CA) for imaging. Solutions were added below the T, and allowed to transition and equilibrated for 5 minutes on the microscope stage. For FRAP experiments, samples (n=3 for each group) were equilibrated for 30 min to prevent thermal movement of the focusing stage, and fluorescence intensity analysis was done using Zen software (ZEISS Microscopy, Jena, Germany). For void volume analysis, 20 μm image stacks (n=3 for each concentration) were taken with a pinhole size of 1 Airy unit and vertical slice intervals of 230 nm. Three dimensional reconstructions of the resultant networks and quantification of their void volume was done in IMARIS 8 (Bitplane. Belfast, Ireland). Surface renders were constructed with a minimum object detail of 200 nm and local background thresholding with the diameter of the largest sphere that fits into the object set a 1 μm. A consistent minimum threshold of 1000 FU was used across samples. Concentrations beyond 800 μM were not evaluated, as quantification using our methodology was not possible as the feature size approached the spatial resolution of the confocal fluorescence microscope. Network fractal dimensions were determined using the 2D box counting algorithm from the FracLac plugin for ImagJ (Schneider, C. A., et al. Nature Methods 2012, 9, 671-675; Karperien, A. FracLac for Image J, version 2.5. Structured illumination microscopy images were taken with assistance from Dr. Kai Wang using an in-house microscope constructed at Janelia Farm in the lab of Dr. Eric Betzig. Technical details and the experimental setup have been previously published (Shao, L., et al. Nat. Methods 2011, 8, 1044-1046). Because the SIM was an upright microscope, polymer was compressed between a glass slide and a coverslip before imaging. All other sample preparation was identical to that for confocal microscopy. Image stacks of 8-12 μm were taken and maximum intensity projections were created and analyzed in ImageJ.

Pharmacokinetic and SPECT analysis. All constructs were endotoxin purified to <1 EU/mL and prepared at 500 μM in sterilized PBS and reacted with ¹²⁵Iodine (Perkin Elmer, Boston MA) in Pierce® pre-coated IODOGEN tubes (Fisher Scientific, Hampton, NH)(Wood, W. G., et al. J. Clin. Chem. Clin. Bio. 1981, 19, 1051-1056). The product was centrifugally purified through 40K MWCO Zeba Spin Desalting Columns (Thermo Scientific, Rockford, IL) at 2500 rpm for 3 min at 4° C. to remove unreacted radioiodine from the conjugate. After labeling, each construct was diluted down to a final biopolymer concentration of 250 μM. The resulting activity dose for the POP was 1.18 mCi mL-1, while the ELP dose was 1.37 mCi mL-1.

Female athymic nude mice were purchased from Charles River and housed in a centralized animal facility at Duke University. All procedures were approved by the Duke University Institutional Animal Care and Use Committee and were in compliance with the NIH Guide for the Care and Use of Laboratory Animals. 50 μL of the POP was prepared in an Eppendorf tube at 63 μCi to provide a reference imaging standard. Prior to either the depot injection, blood draw, or single-photon emission computed tomography (SPECT) imaging, each mouse was anesthetized using a 1.6% isoflurane vaporizer feed at an O₂ flow rate of 0.6 L min⁻¹. For depot injections, each mouse received a soluble 200 μL injection of their respective solution at 250 μM into the subcutaneous space on the right hind flank. The whole body activity of the mouse was then measured in an AtomLab 400 dose calibrator (Biodex, Shirley, NY). A total of 12 athymic nude mice (n=6 for each group) were used for pharmacokinetic analysis of depot stability and distribution. An initial 10 μL blood sample was drawn and pipetted into 1000 mg mL⁻¹ heparin with subsequent blood draws at time points of 45 min, 4 h, 8 h, 24 h, 48 h, 72 h, 96 H, and 120 h to determine the release profile for the depots. 6 total athylmic nude mice also were imaged using SPECT at time points of 0, 48, and 120 hrs. Mice were then transferred under anesthesia to the bed of the U-SPECT-II/CT for imaging using a 0.350 collimator (MILabs B.V., Utrecht, Netherlands) courtesy of G. Al Johnson in the Duke CIVM. Anesthesia was maintained with a 1.6% isoflurane feed at an O₂ flow rate of 0.6 L min⁻¹. SPECT acquisition was conducted over a time frame of 15 minutes in ‘list-mode’ and at a ‘fine’ step-mode. Upon completion, a subsequent CT scan was carried out at a current of 615 μA and a voltage of 65 kV. Mice were then returned to their cages. Post-imaging SPECT reconstruction was carried out using MILabs proprietary software without decay correction and centered on the ¹²⁵I photon range of 15-45 keV. All images were reconstructed at a voxel size of 0.2 mm. Reconstructed SPECT images were then registered with their corresponding CT scans to provide spatial alignment for anatomical reference.

Upon completion of the study, all mice were euthanized and dissected. The subcutaneous depots were excised and visually examined for physical differences. In addition, the heart, thyroid, lungs, liver, kidneys, spleen, skin, muscle and pancreas were collected and analyzed using a Wallac 1282 Gamma Counter (Perkin Elmer, Boston, MA) to determine the relative biodistribution of the different constructs. All blood samples and the set of PK standards were similarly analyzed using the gamma counter. The counts per minute detected for each sample were converted to their corresponding activity. Blood samples were then scaled to determine the total amount in circulation according to the formula Total=CPM/.01*BW*72 mL/kg (Diehl, K. H. et al. J. Appl. Toxicol. 2001, 21, 15-23). Depot retention was analyzed by measuring the total photon intensity of the depot SPECT image in ImageJ. Measured photon intensity was converted to total depot activity using a calibration factor determined from the imaging standard. This calibration was determined by performing a linear regression of the known activities of the standard over time against the corresponding SPECT intensity measurements. The factor was applied to each depot and the calculated activity compared against the original whole body injected dose at 0 h to determine its percent retention.

Cell Recruitment. Female C57BL/6 mice were purchased from Charles River and housed in a centralized animal facility at Duke University. All procedures were approved by the Duke University Institutional Animal Care and Use Committee and were in compliance with the NIH Guide for the Care and Use of Laboratory Animals. For analysis of POP persistence and cell recruitment, female C57BL/6 mice (used for their complete immune system over the nude mice used in the previous study for easier depot observation) received soluble injections in the subcutaneous space of the right and left hind flanks. Mice were injected with either 200 μL of 250 μM E1-H5-25%-120 (19), 200 μL of 750 μM E1-H5-25%-120 (4), or Matrigel (Standard Formulation, Corning, Tewksbury, MA) (7). Matrigel was chosen for comparison since fully disordered ELPs were shown to dissipate too quickly for long term analysis of cell recruitment. POPs were endotoxin purified to <1 EU/mL and sterile filtered prior to injection. At respective time points, mice were euthanized and dissected. Left hind injections were excised and placed in 10% neutral buffered formalin (Sigma, St. Louis, MO) for histological analysis (n=3 for all groups). Fixed depots were embedded in paraffin, and 5 um slices from the center of each depot were stained with Hematoxylin and Eosin (H&E). H&E stained slides were imaged using an Axio 506 color camera mounted on a Zeiss Axio Imager Widfield microscope. Images at 200× magnification were stitched and exported for analysis. Blood vessels and capillaries were manually counted in ImageJ (n=3). For changes in depot size, images of excised depots were taken at a controlled distance and imported into ImageJ for quantification.

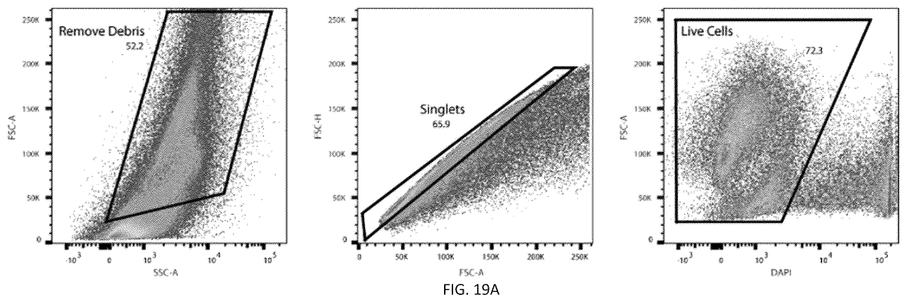

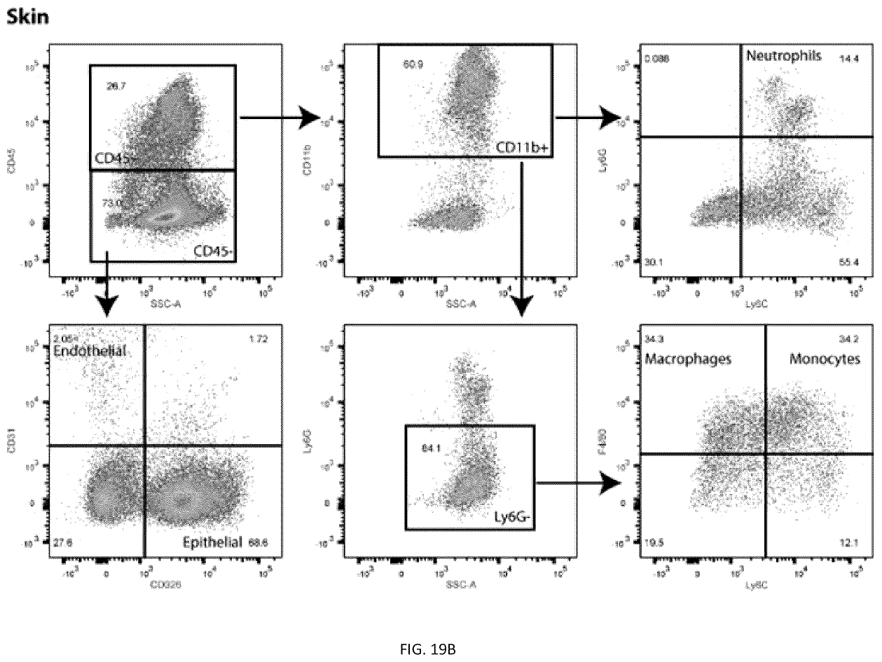

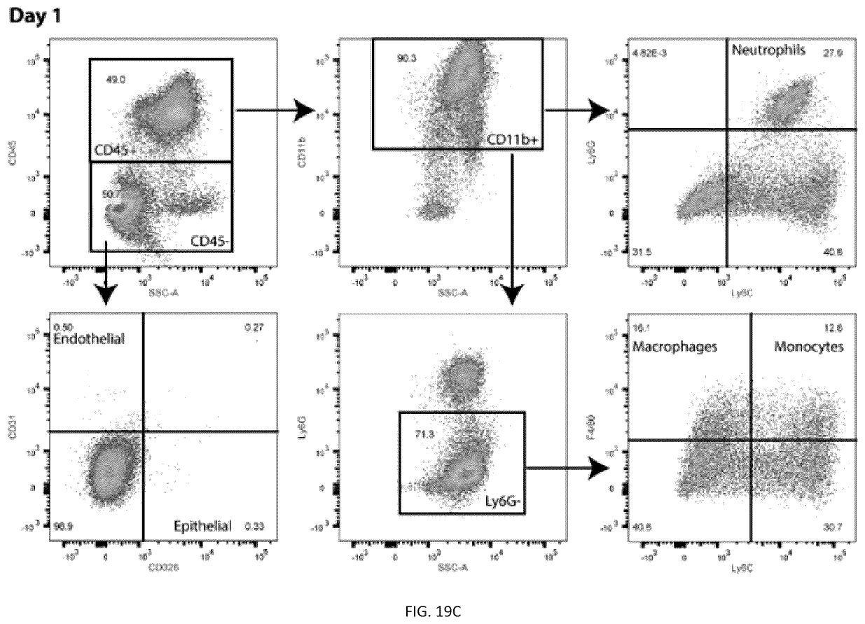

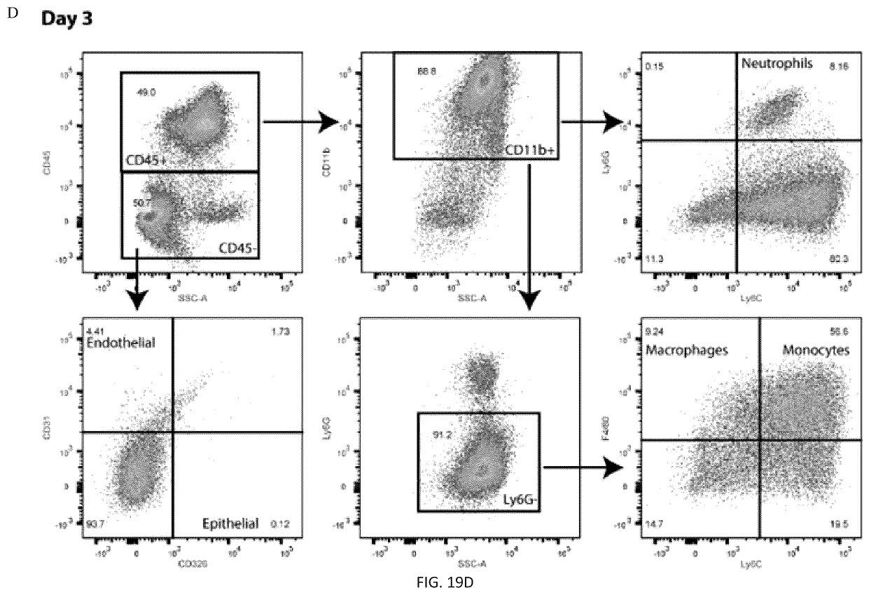

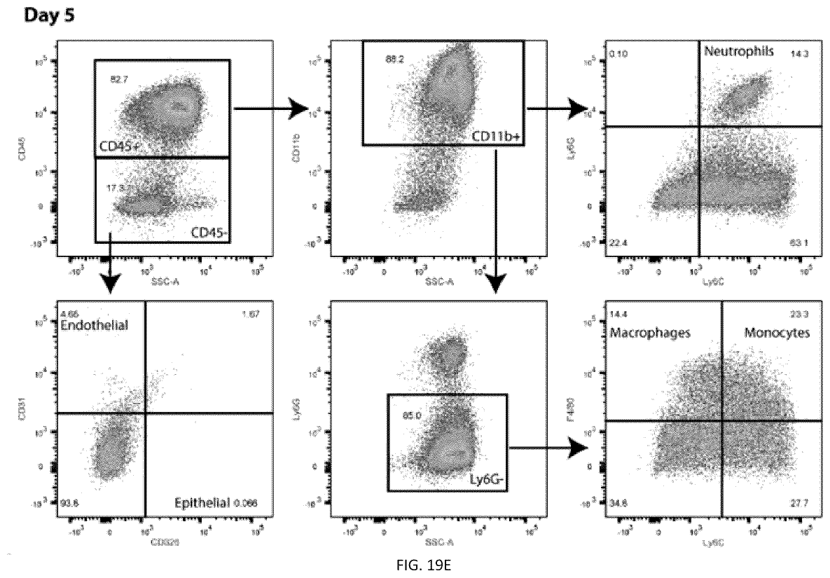

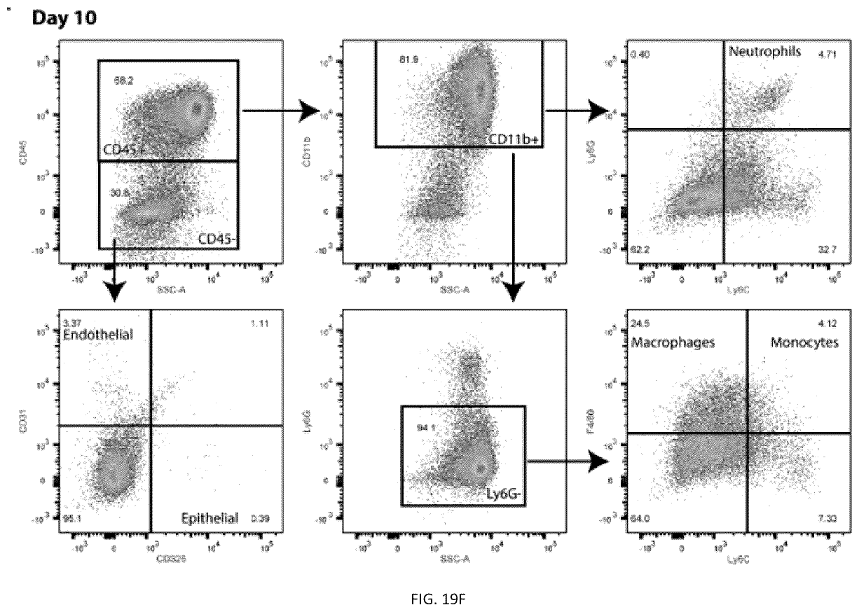

For flow cytometry, excised right hind injections (n=3-4) were transferred to 2 mL PBS and digested with 0.5 mg/ml Collagenase IV (Sigma, St. Louis, MO) and 50 units of DNase I (Sigma, St. Louis, MO) at 37° C. for 45 min. Digested tissue was filtered through a 70 μm cell strainer and repeatedly washed with sterile 2% FBS (Thermo Fisher, Waltham, MA) in PBS. After ACK (Thermo Fisher, Waltham, MA) lysis, cells were counted using a hemocytometer. Cells were stained as previously described (Chen, J. et al. Biomaterials 2013, 34, 8776-8785). Briefly, cells were blocked with 2.4 G2 antibody (BD Biosciences, San Jose, CA) and stained with anti-mouse CD45-BV-510 (30-F11, BD Biosciences, San Jose, CA), F4/80-PerCP-Cy5.5 (BM8, Biolegend, San Diego, CA), CD11b-APC (M1/70, Biolegend, San Diego, CA), Ly6C-FITC (AL-21 Biolegend, San Diego, CA), Ly6G-PE (IA8, Biolegend, San Diego, CA), CD31-PE-Cy7 (390, Biolegend, San Diego, CA), CD326-APC-Cy7 (G8.8, Biolegend, San Diego, CA), and DAPI (Biolegend, San Diego, CA). Cell subtypes were defined as: neutrophils (CD45+CD11b+Ly6C+Ly6G+F4/80−), inflammatory monocytes (CD45+, CD11b+F4/80+Ly6C+Ly6G−), tissue macrophages (CD45+CD11b+F4/80+Ly6C−Ly6G−), endothelial cells (CD45−CD31+CD326−), and epithelial cells (CD45−CD326+CD31−). Cell type analysis was done on a FACSCANTO II (BD Biosciences, San Jose, CA) with a minimum of 50000 live cells analyzed for each sample. AbC anti-rat antibody control beads (Thermo Fisher, Waltham, MA) were used as compensation controls for all antibody dyes, and a 1:1 mixture of live and dead cells was used as a compensation control for DAPI. Gating procedures are shown in FIG. 19. Flow cytometry data was gated and quantified with FlowJo (FlowJo LLC, Portland, Oregon) and exported for analysis.

Statistical Analysis. All statistical analysis was carried out using Prism 6 (Graphpad Inc, La Jolla, CA). When comparing individual groups, two-tailed t-tests were used to determine statistical significance. ANOVA was used to evaluate significance among three or more groups and the Tukey-Kramer method was used as a post hoc test for comparisons between groups. Experimental group sizes are given within the descriptions of each experiment.

### Example 2

### Polymer Library Design

We chose elastin-like polypeptides (ELPs), a family of repetitive polypeptides based on a consensus (VPGXG) pentapeptide repeat derived from the disordered regions of tropoelastin and that exhibit a tunable lower critical solution temperature (LCST) phase behavior (McDaniel, J. R., et al. Biomacromolecules 2013, 14, 2866-2872; Li, N. K., et al. Biomacromolecules 2014, 15, 3522-3530; Meyer, D. E. & Chilkoti, A. Biomacromolecules 2004, 5, 846-851; Roberts, S., et al. FEBS Lett. 2015, 589, 2477-2486) as our disordered component. ELPs have been characterized as models of elastomeric disorder and their intrinsic disorder is thought to be at least partially responsible for their LCST behavior. They are biocompatible polymers with numerous applications including protein purification, drug delivery, and tissue engineering. Polyalanine helices are also an important element of tropoelastin where they combine with disordered domains to produce the elasticity and resilience that make elastin an important component of the extracellular matrix. In aqueous solutions, alanine-rich sequences are known to have high intrinsic alpha-helical propensity, and they can drive self-association through intermolecular interactions either via helical bundling or the formation of beta-sheet rich fibrils. Accordingly, we selected polyalanine as the scaffold for the ordered domains. We hypothesized that recombinant polymers composed of polyalanine domains doped into an ELP scaffold, thus mimicking the exon composition and organization of tropoelastin, would produce biomaterials with unique, tunable properties.

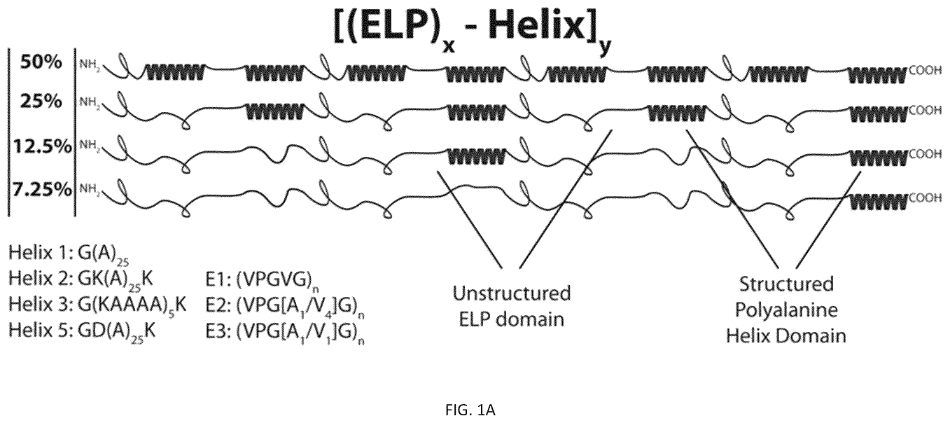

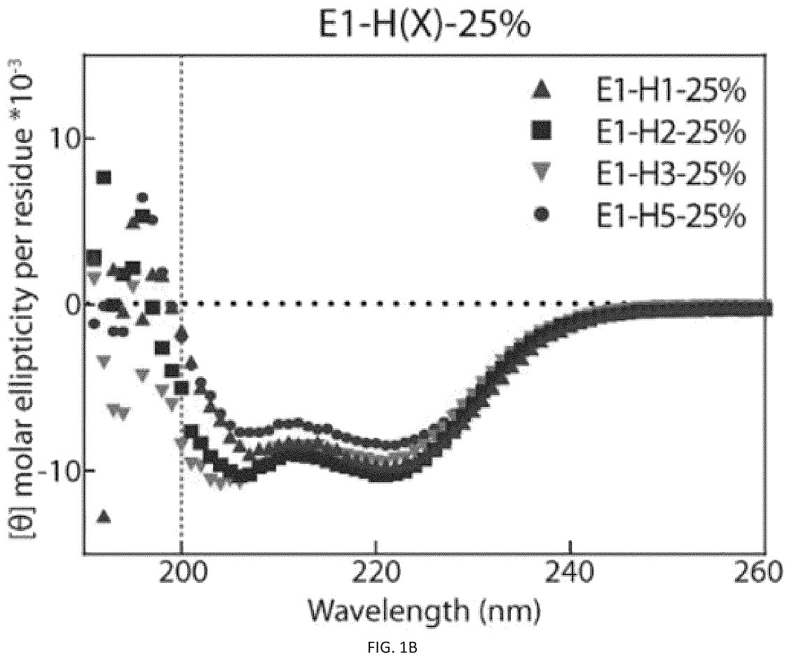

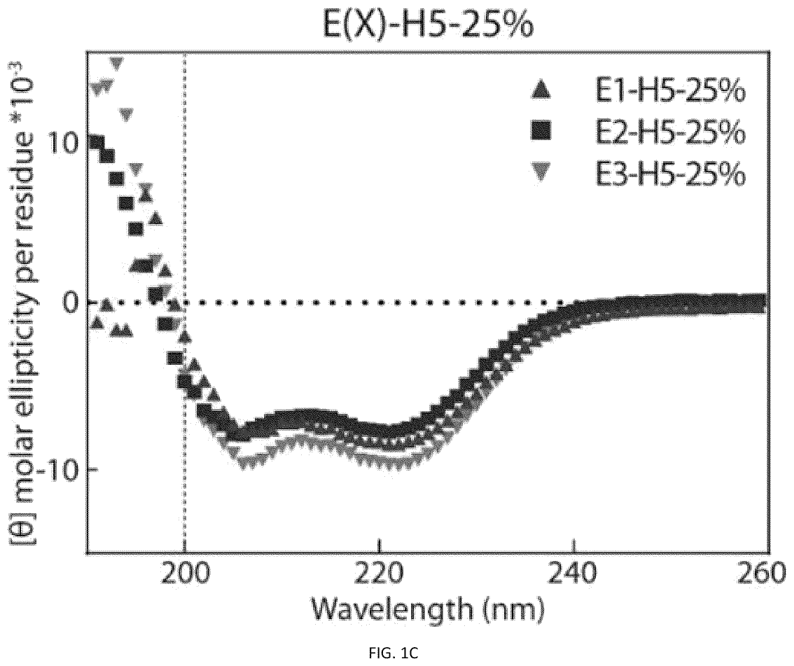

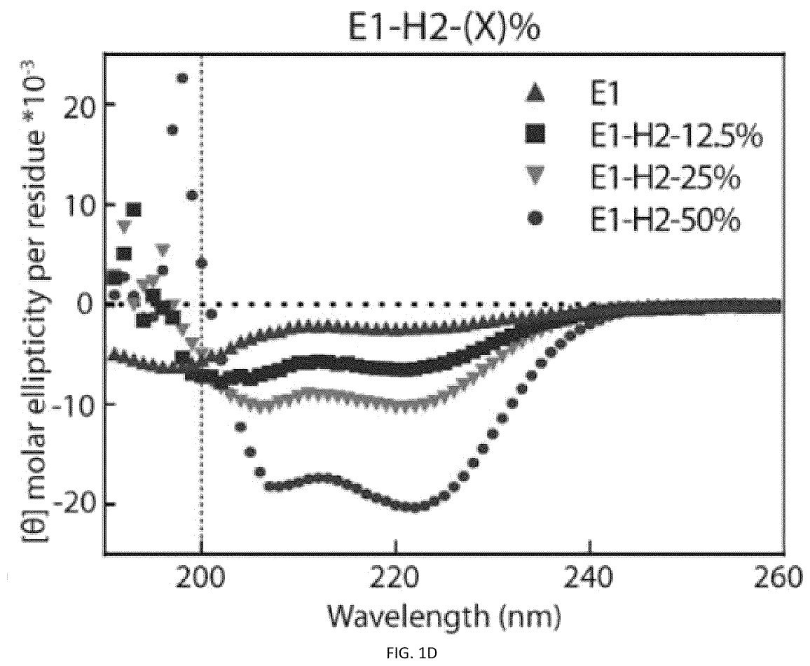

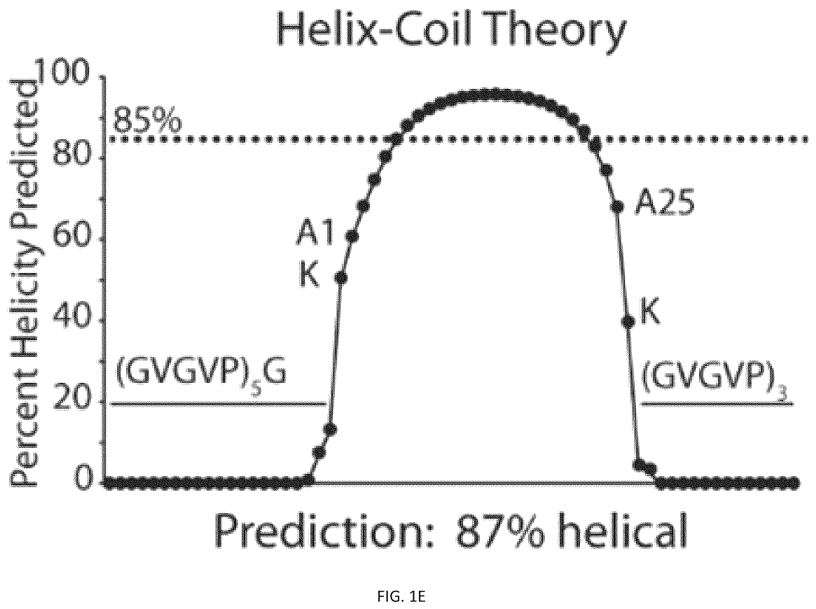

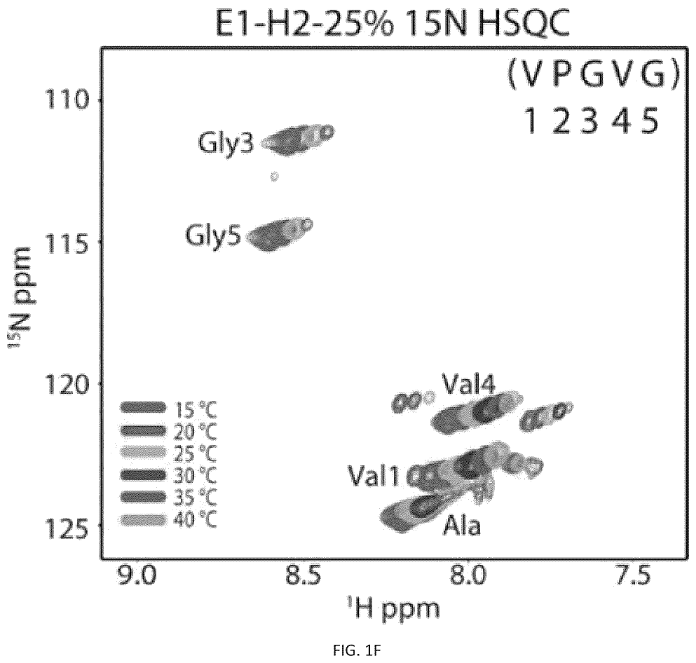

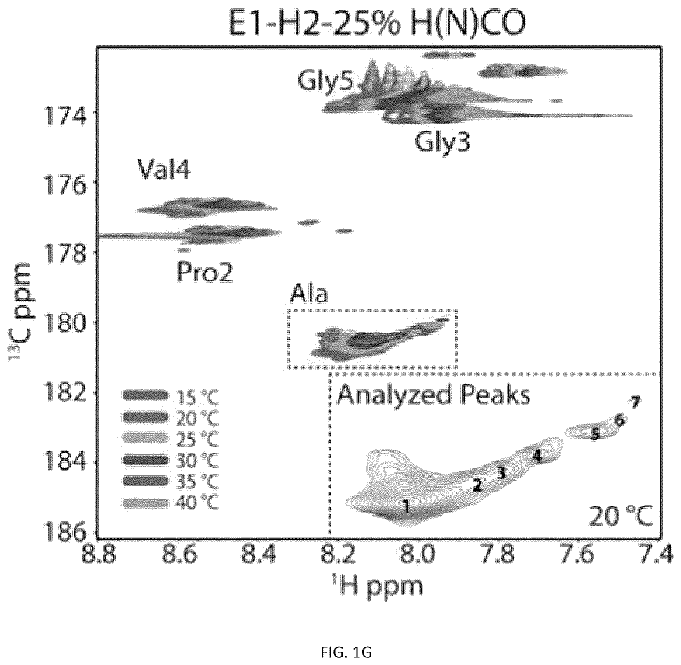

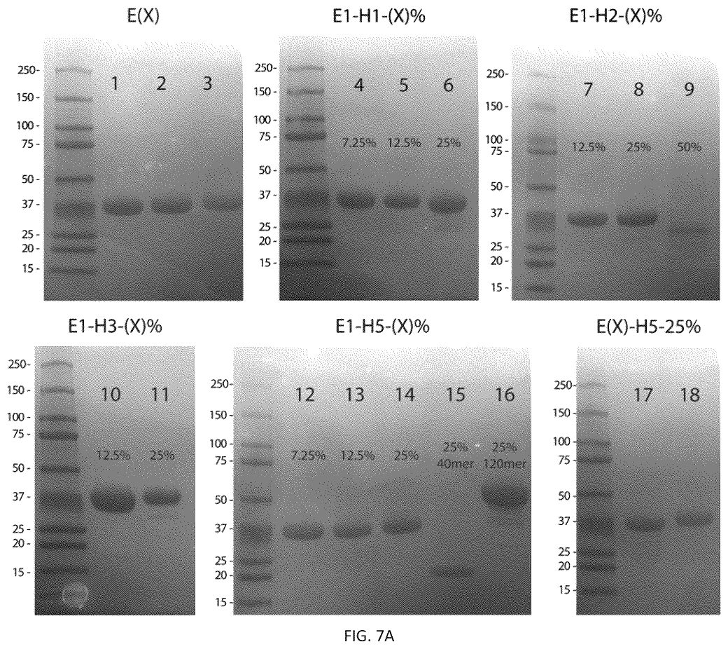

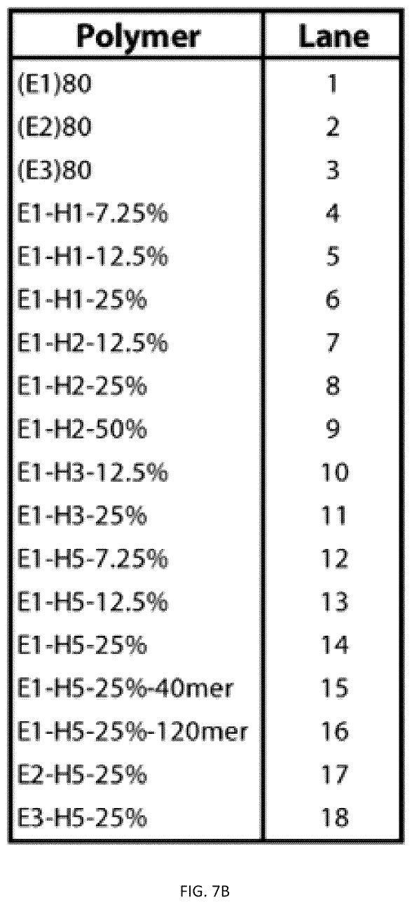

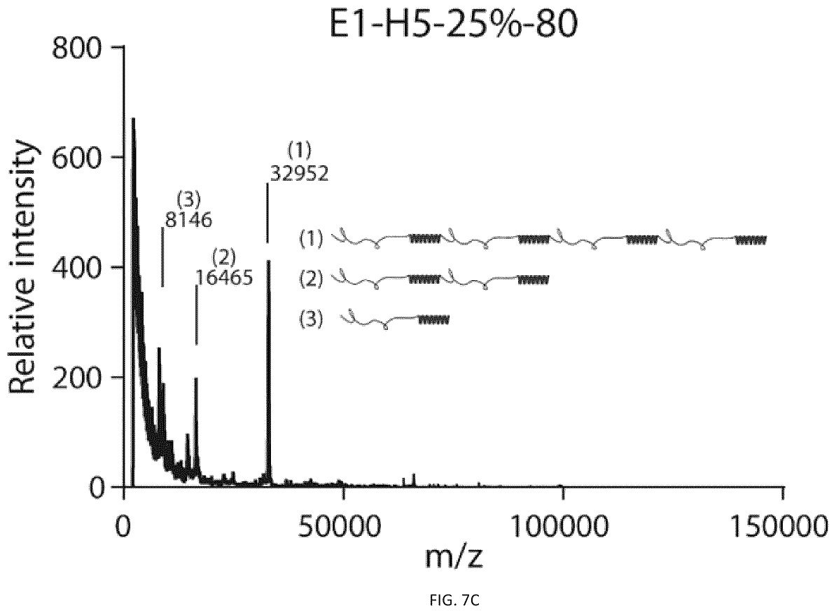

Four polyalanine helices (H1, 2, 3, and 5) with different charge distributions were incorporated into three ELPs (E1-3) of varying side chain hydrophobicities at either 7.25%, 12.5%, 25%, or 50% of the total amino acid number (FIG. 1A). Polyalanine domain compositions were chosen to maximize helicity while controlling hydrophilicity through charge-charge interactions. ELP compositions were chosen to span a range of LCSTs suitable for in vivo injection. The naming convention for our partially ordered polymers (POPs) specifies the ELP (EX), the helix (HY), and the percent helicity (Z %): EX-HY-Z %. The molecular weights (MW) for all polymers are in TABLE 1. All POPs and ELP controls were recombinantly expressed from plasmid-borne synthetic genes in E. coli and purified to >95% by inverse transition cycling (Meyer, D. E. & Chilkoti, A. Nat. Biotechnol. 1999, 17, 1112-1115). POP purity and homogeneity was verified by sodium dodecyl sulfate polyacrylamide gel electrophoresis (SDS-PAGE) (FIG. 7), and their MWs were verified by matrix assisted laser desorption ionization mass spectrometry (MALDI-MS); in all cases, the experimentally determined MWs agreed with their theoretical MWs within 2%.

TABLE 1
Polymer lengths and molecular weights.

| Polymer | AA Number | MW (kDa) |
|---|---|---|
| (E1)80 | 404 | 33.2 |
| (E2)80 | 404 | 32.8 |
| (E3)80 | 404 | 32.1 |
| E1-H1-7.25% | 405 | 33.0 |
| E1-H1-12.5% | 406 | 32.8 |
| E1-H1-25% | 408 | 32.4 |
| E1-H2-12.5% | 410 | 33.3 |
| E1-H2-25% | 416 | 33.4 |
| E1-H2-50% | 428 | 33.6 |
| E1-H3-12.5% | 408 | 33.6 |
| E1-H3-25% | 412 | 34.0 |
| E1-H5-7.25% | 407 | 33.2 |
| E1-H5-12.5% | 410 | 33.3 |
| E1-H5-25% | 416 | 33.4 |
| E1-H5-25%-40mer | 210 | 16.9 |
| E1-H5-25%-120mer | 622 | 49.8 |
| E2-H5-25% | 416 | 33.0 |
| E3-H5-25% | 436 | 34.1 |

*Met leader and Gly-Try-Pro trailer for all polymers

### Example 3

### Structural Characterization

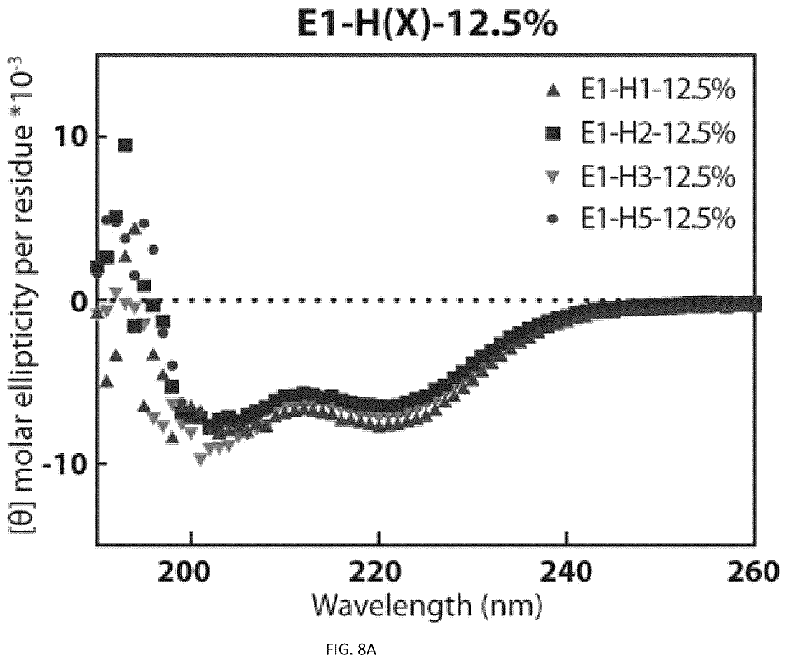

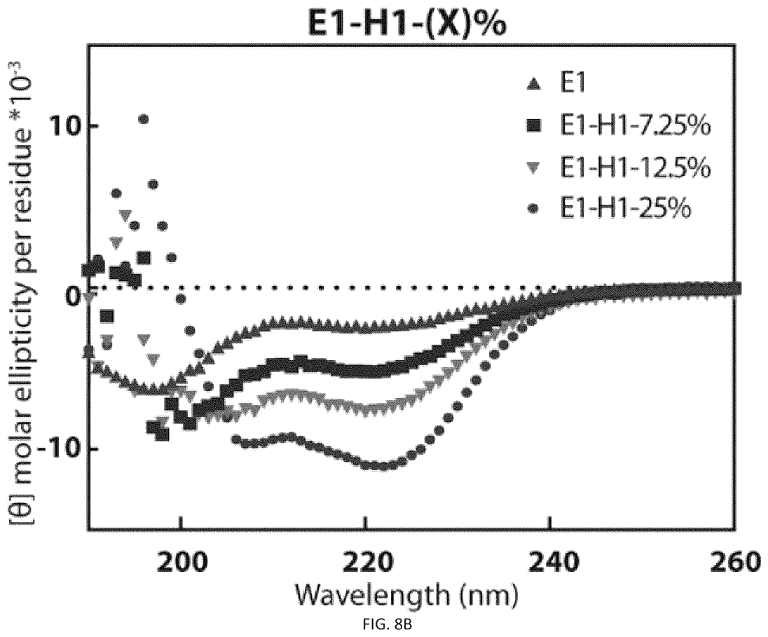

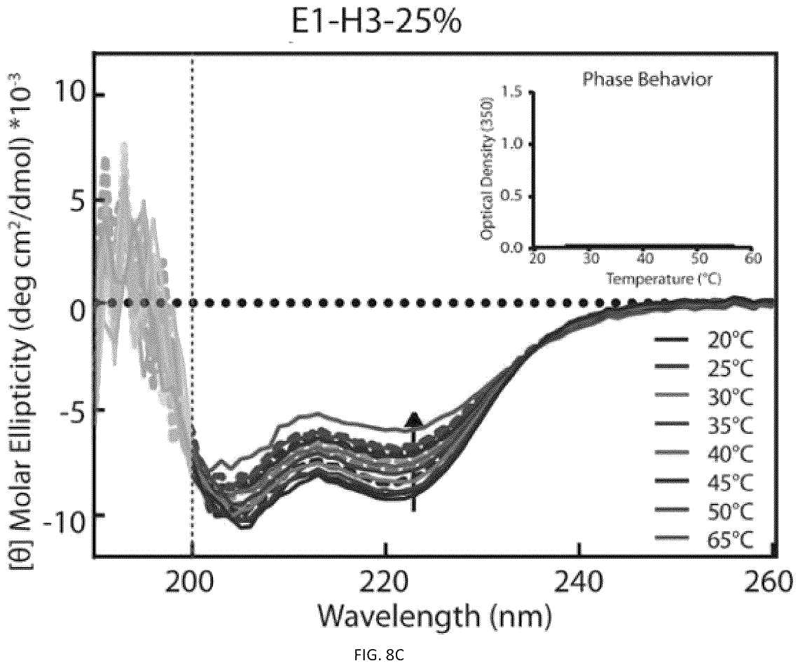

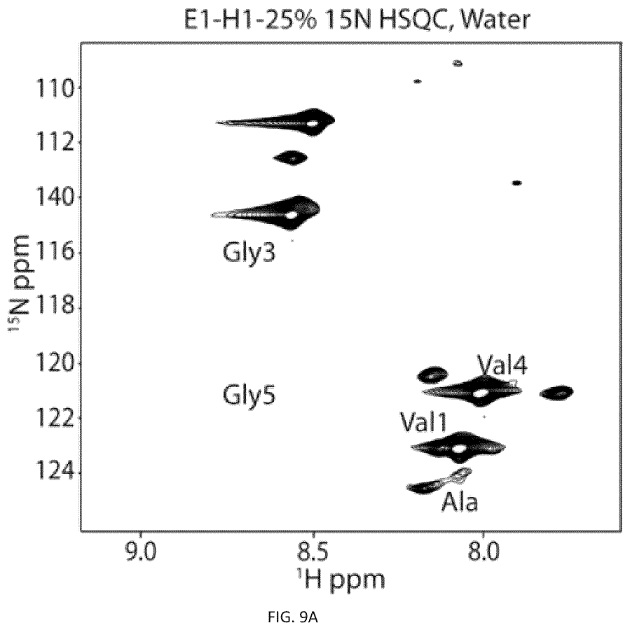

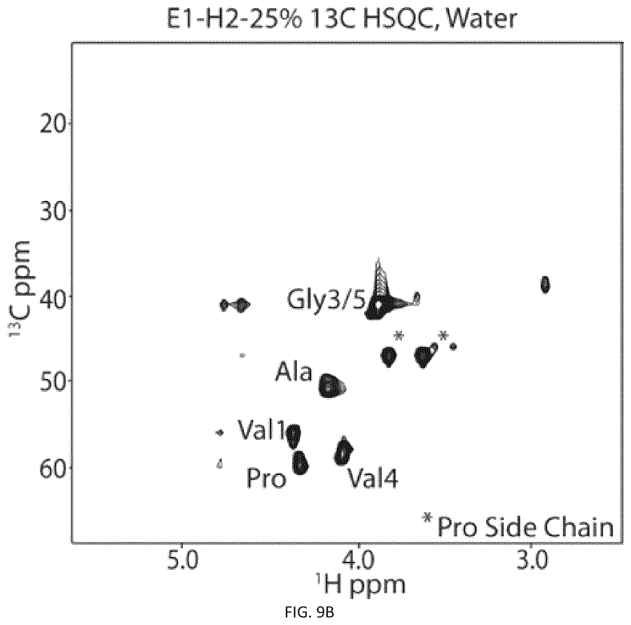

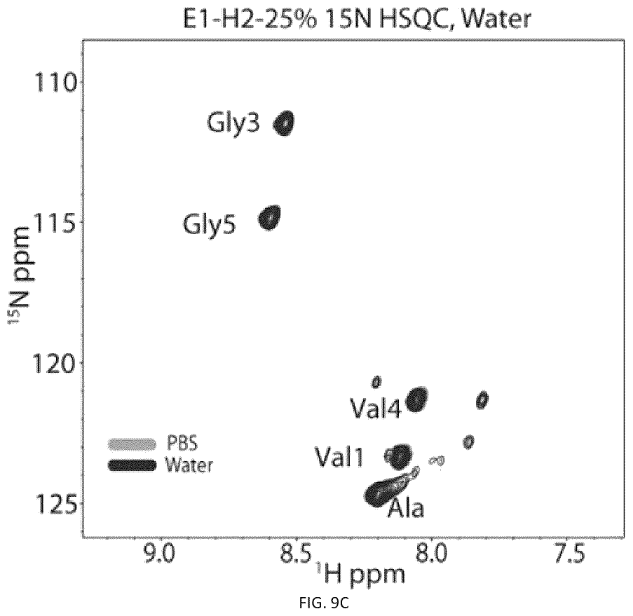

We used ultraviolet circular dichroism (UV-CD) to verify the secondary structure of POPs. All POPs show the negative ellipticity peaks at 222 nm and 208 nm (FIG. 1B-FIG. 1D and FIG. 8). These bands are characteristic of α-helices. Peak magnitudes are largely independent of polyalanine and ELP composition but are highly dependent on total polyalanine percentage. The helices are thermally stable with minimal melting at temperatures of up to 65° C. (FIG. 8). Helicity was quantified using BeStSel (TABLE 2)(Micsonai, A. et al. Proc. Natl. Acad. Sci. U.S.A. 2015, 112, E3095-3103); however, because quantitative analysis of UV-CD data for disordered proteins can be inaccurate, we also used 2D-solution NMR to determine POP helicity. Though the repetitive and proline rich nature of POPs increases the complexity of resonance assignments, identifying key amino acids was still feasible using combinations of triple resonance NMR spectra (FIG. 1F-FIG. 1G and FIG. 9). Based on the backbone carbonyl carbon chemical shifts of the alanine peaks in the H(N)CO spectrum—a particularly sensitive spectral signature for secondary structure changes—90% of the residues within each polyalanine domain (H2) were found to be in a helical conformation at 20° C. This result is supported by predictions from helix-coil transition theory (FIG. 1E) (Muñoz, V. & Serrano, L. Nature Structural Biology 1994, 1, 399-409; Munoz, V. & Serrano, L. Journal of Molecular Biology 1995, 245, 275-296; Munoz, V. & Serrano, L. J. Mol. Biol. 1995, 245, 297-308) and the temperature dependent change of the chemical shifts of backbone carbonyl carbons (TABLE 3). Given the similarity in UV-CD structural signatures (FIG. 1B), the remaining helical compositions can be confidently approximated to a similar degree of structure.

TABLE 3
NMR peak predicted helicity and temperature shift coefficients.

| Peak | Helicity* | Coefficient (ppb/K)** |
|---|---|---|
| 1 | 93.0% | −19.6 |
| 2 | 91.9% | −20.8 |
| 3 | 90.9% | −20.7 |
| 4 | 89.4% | −24.2 |
| 5 | 86.0% | −25.7 |
| 6 | 84.1% | −29.1 |
| 7 | 79.7% | −30.3 |

*determined for H2 at 20° C. based on chemical shifts at 20° C.
**¹³C in H(N)CO spectra

### Example 4

### Sharp Phase Behavior and Tunable Hysteresis

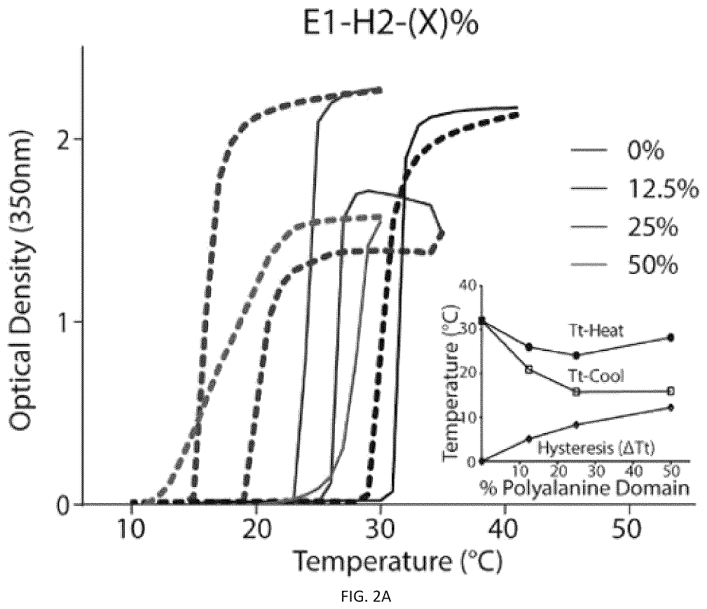

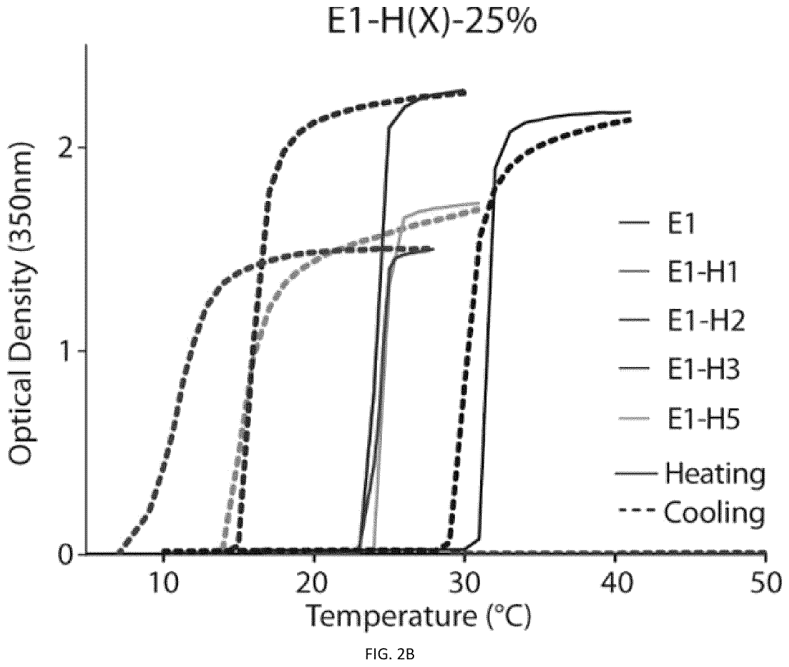

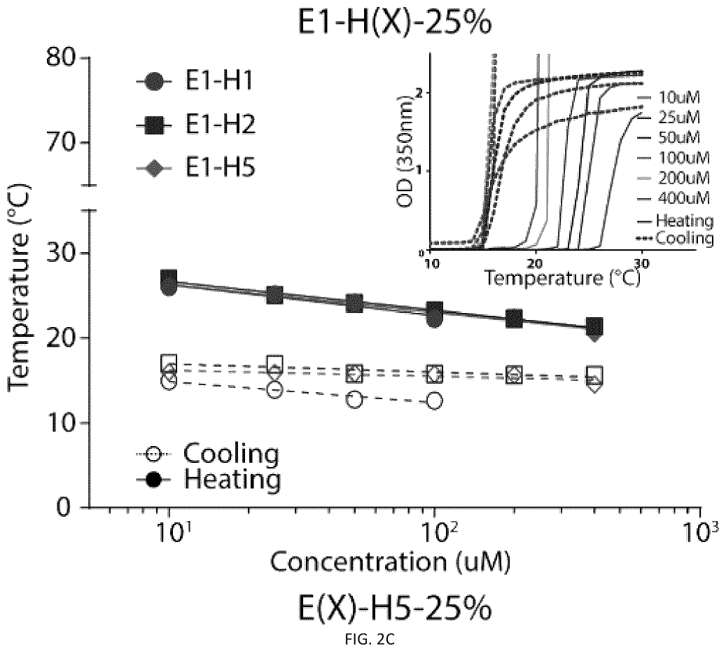

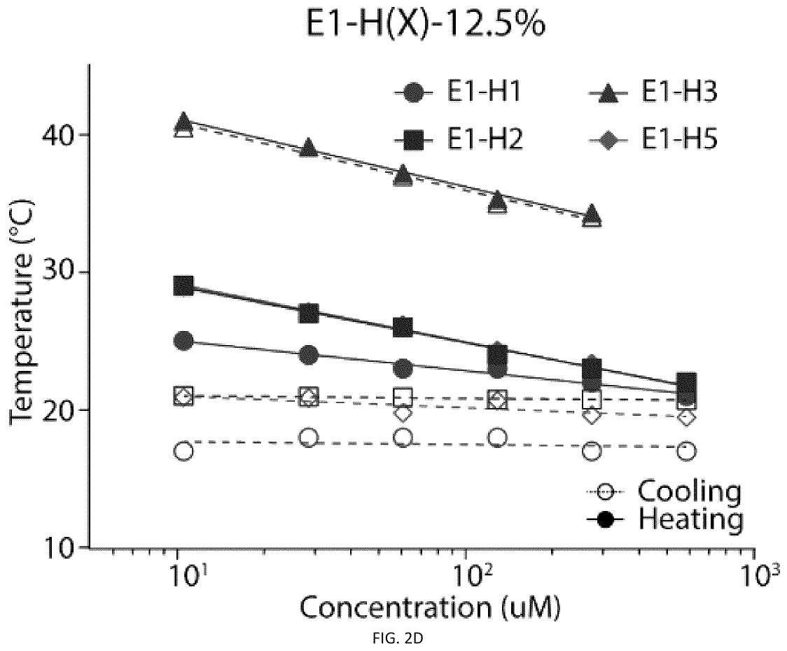

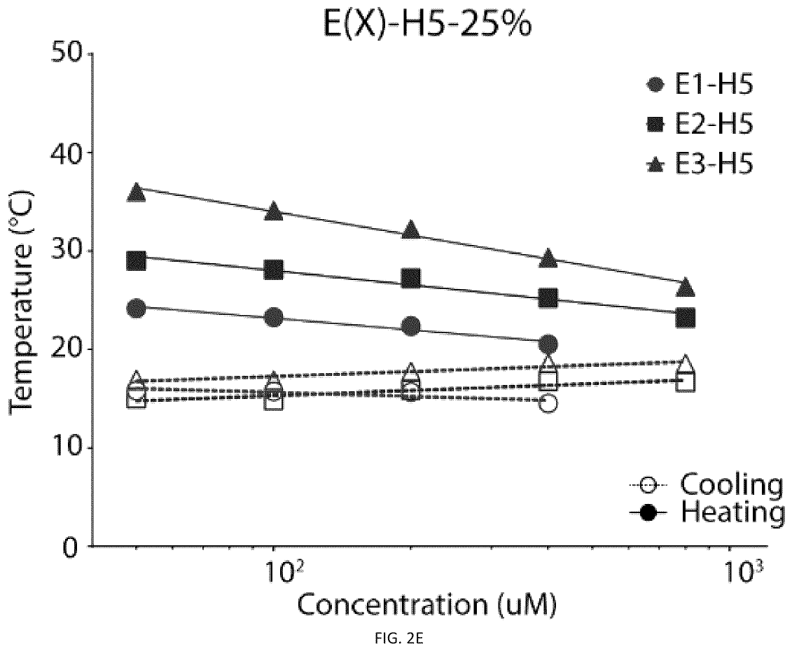

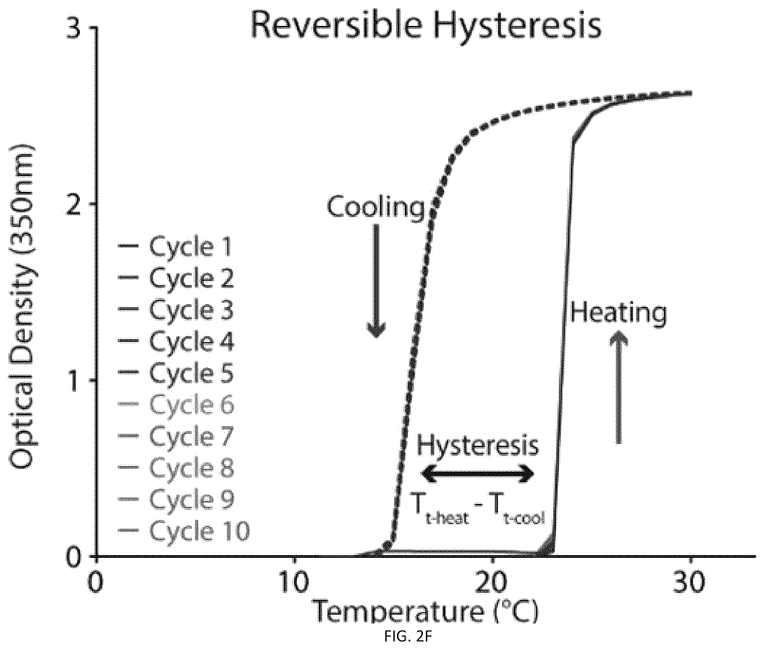

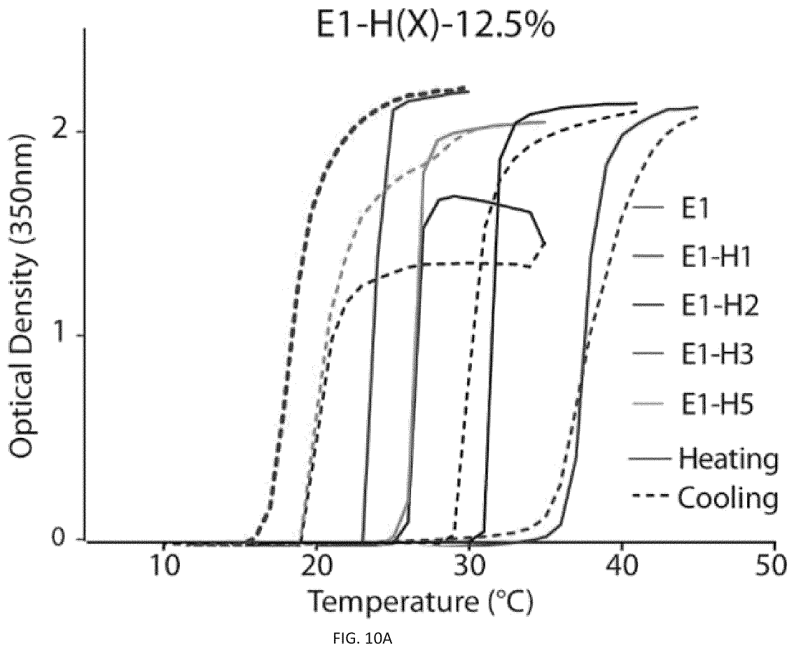

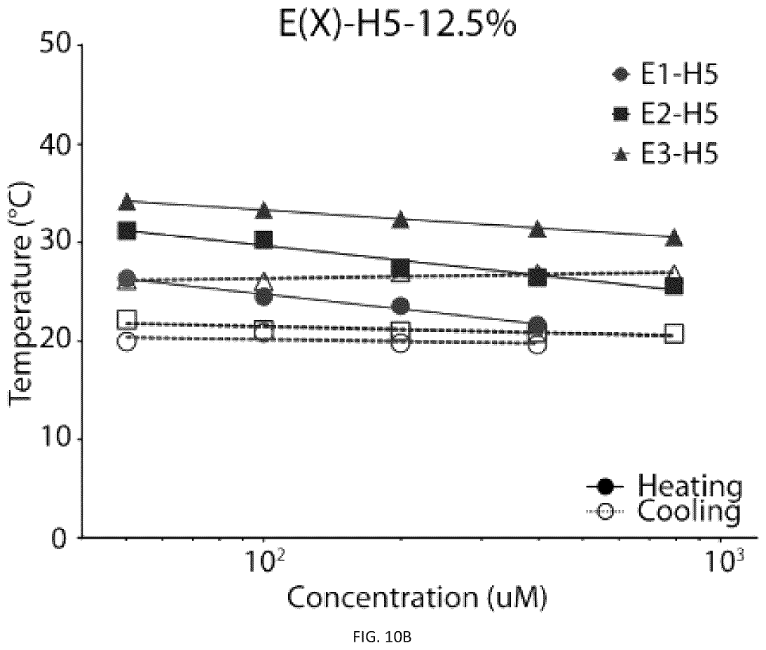

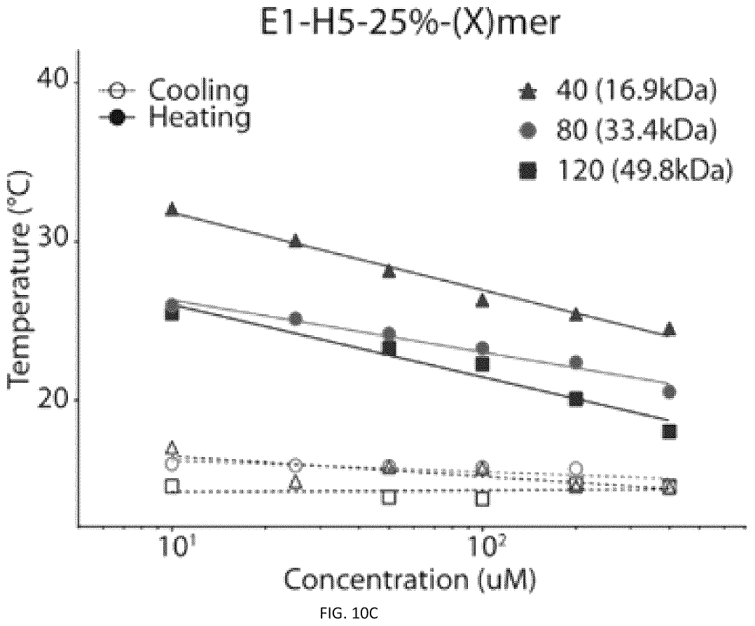

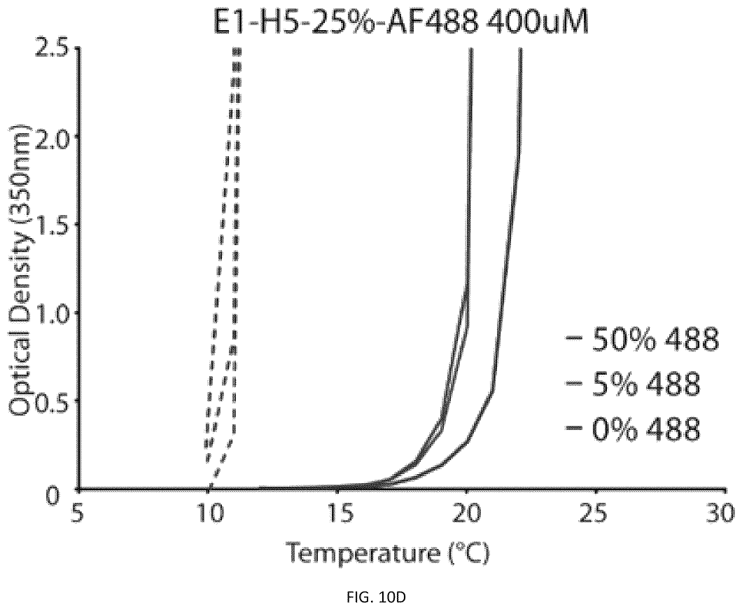

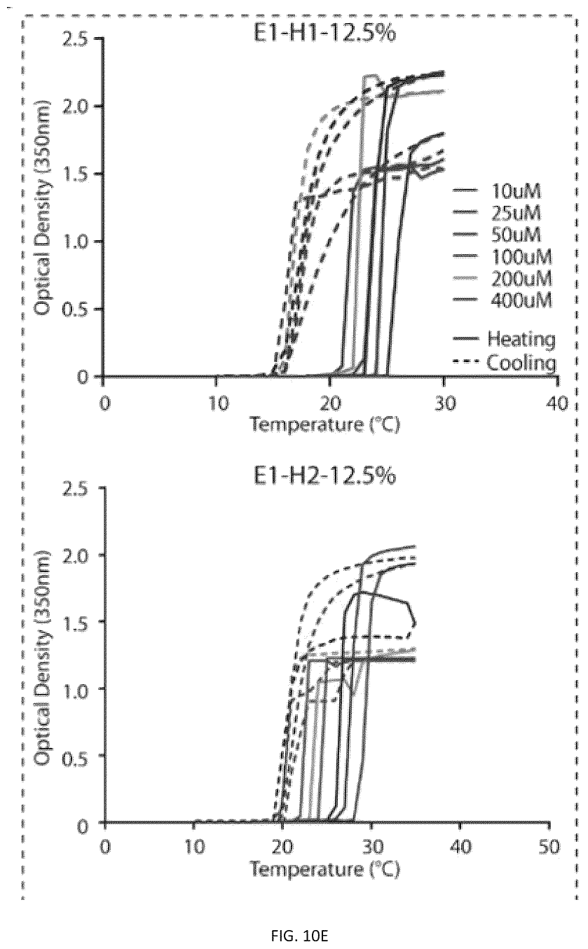

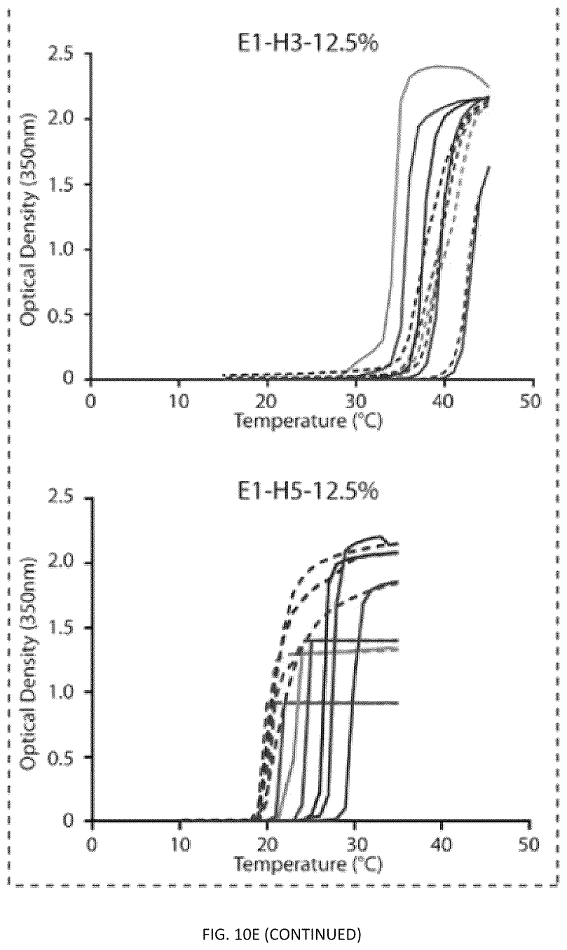

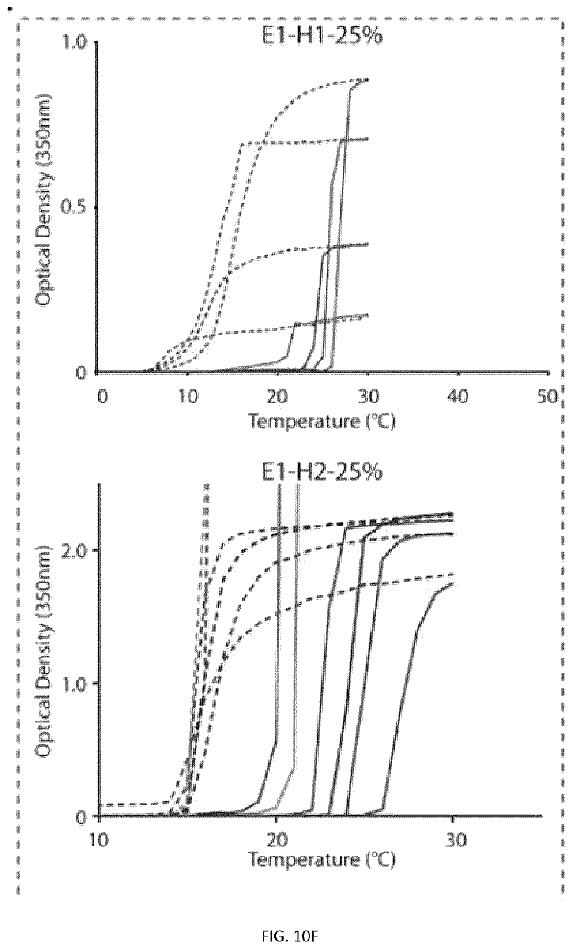

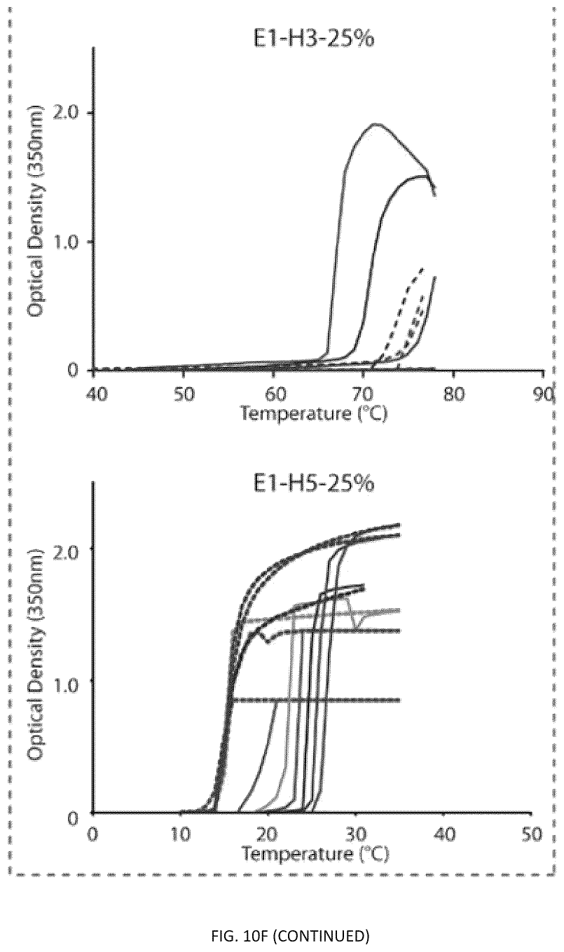

ELPs exhibit thermally reversible LCST behavior, cycling between clear solutions and turbid states. We measured the thermal phase transition of our POPs by monitoring their optical turbidity as a function of temperature. Remarkably, all proteins demonstrate very sharp phase transitions that occur over a 1-2° C. range, even when composed of 50% α-helix (FIG. 2 and FIG. 10). These transition temperatures vary depending on the specific ELP and helix composition due to differences in their hydrophilicity and charge, but all POPs exhibit the sharp phase behavior characteristic of fully disordered ELPs.

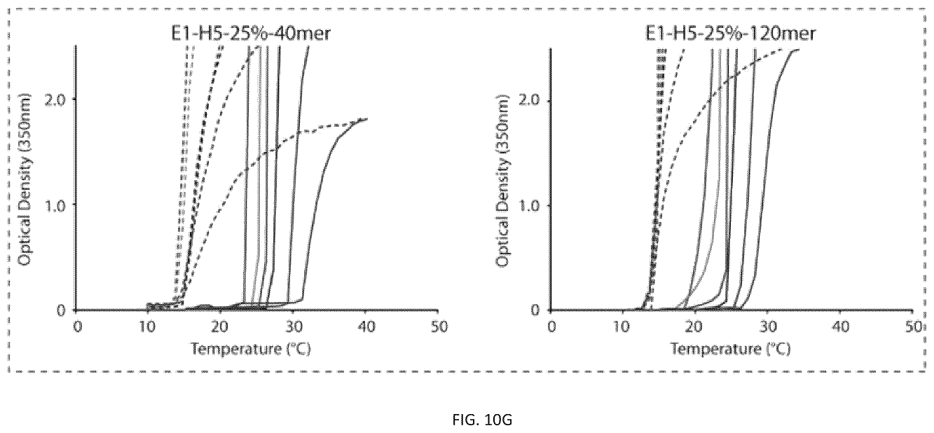

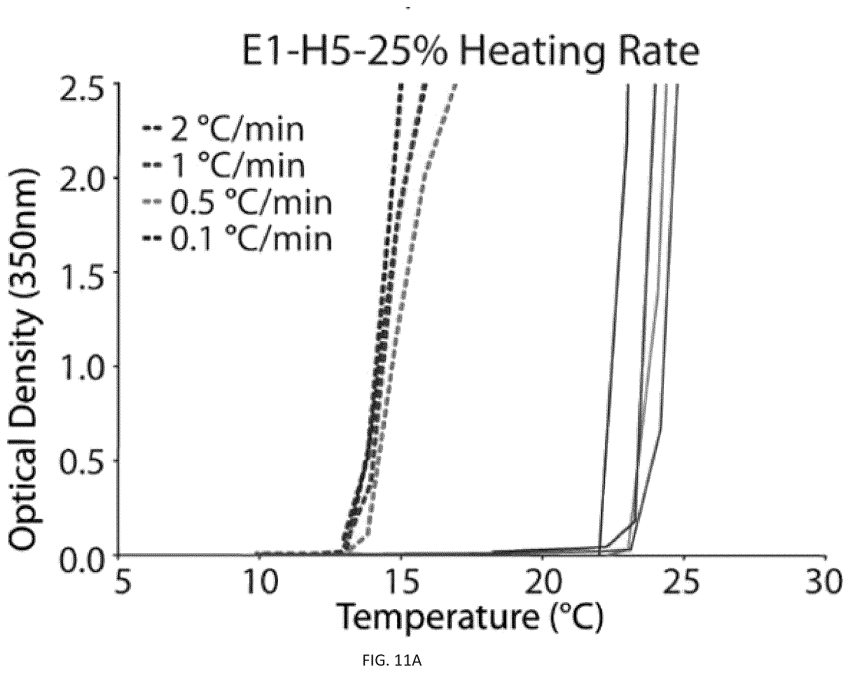

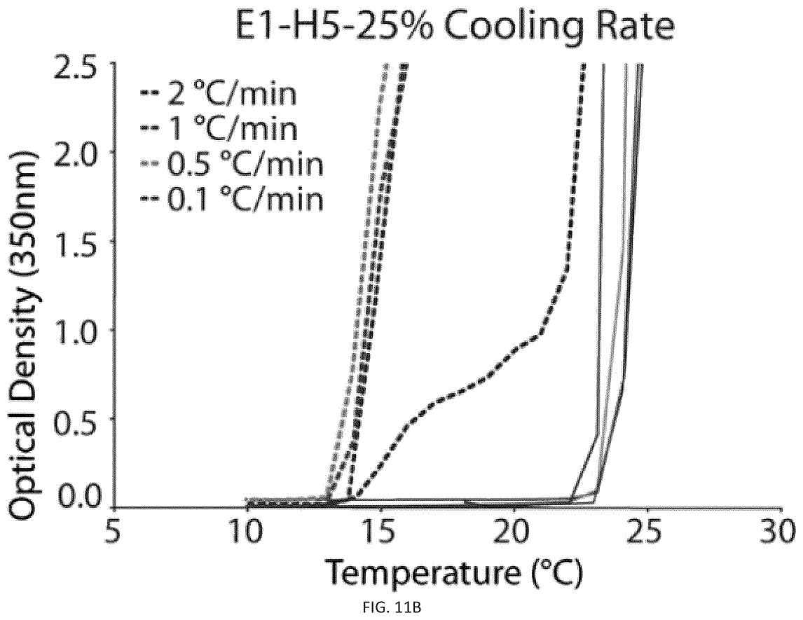

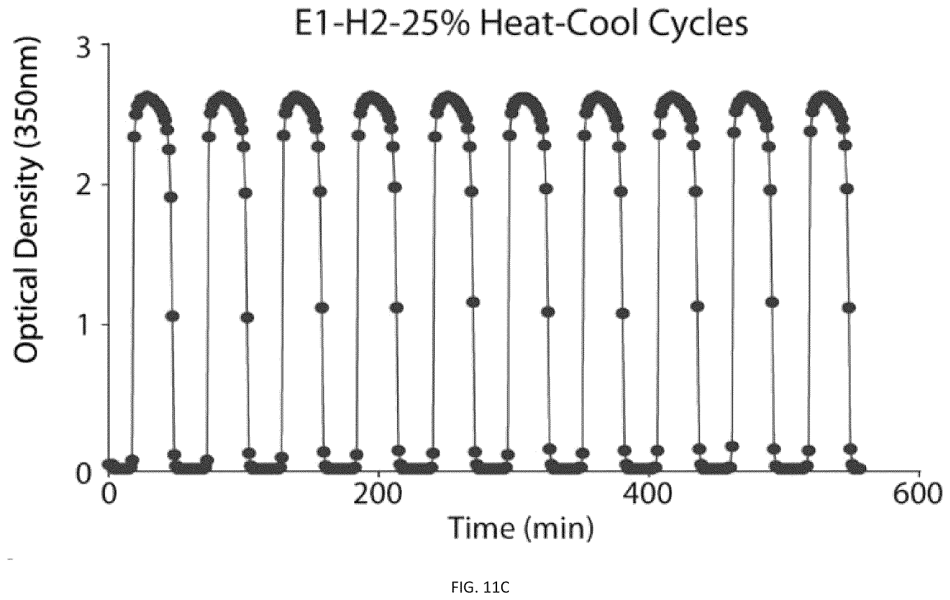

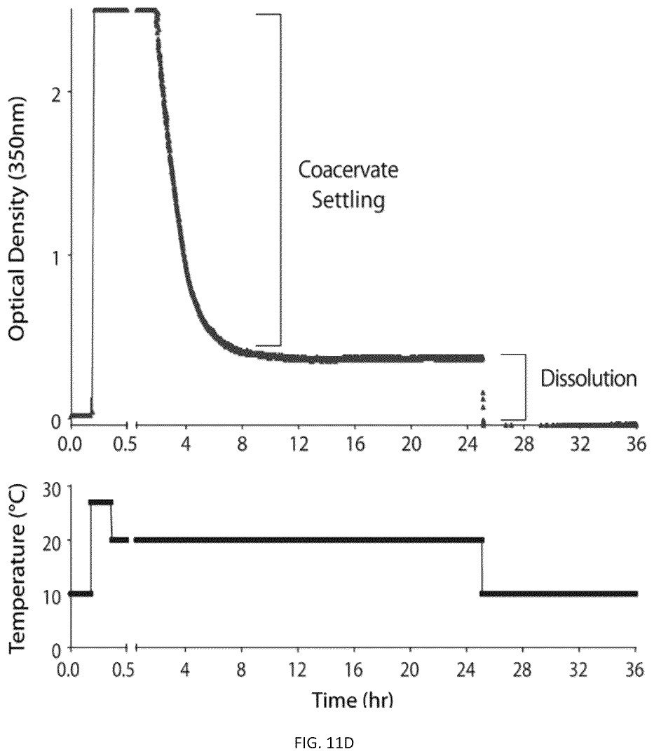

When turbid POP solutions are cooled, they form clear solutions; however, one aspect their behavior was of particular interest—the marked downshift in the Tₜ-cooling from the original Tₜ-heating. This thermal hysteresis, defined as the difference between Tₜ-heating and Tₜ-cooling (ΔTₜ), is not observed in ELPs although it has been advantageous in other recombinant polymers for the development of hyper-stable nano/micro-particles and for stabilizing protein scaffolds. However, the inability to tune the temperature range over which hysteresis occurs in these systems has severely impeded their application. In contrast, the thermal hysteresis in POPs can be precisely controlled as it directly correlates with polymer helicity (FIG. 2A) and inversely correlates with the amount of charge on the helix side chains (FIG. 2B-FIG. 2D and FIG. 10). Importantly, once fully solvated, POPs return to their original state and can be cyclically heated and cooled with no permanent alterations (FIG. 2F). By incorporating helices with sufficient charge repulsion, such as H3, hysteresis can be eliminated altogether. Hysteresis is independent of both heating and cooling rates, and polymers heated and then cooled to their hysteretic range remain aggregated after 24 h (FIG. 11). Subsequent cooling below the Tₜ-cooling after 24 h causes rapid dissolution.

For POPs, The Tₜ-heating scales logarithmically with polymer concentration, in accordance with ELP behavior. However, Tₜ-cooling is independent of concentration (FIG. 2C-FIG. 2E). Altering the ELP composition adjusts the Tₜ-heating appropriately, but does not change the Tₜ-cooling (FIG. 2E). These observations indicate that the Tₜ-heating is controlled by the composition and chain length of the ELP segment, while the helix composition is the primary determinant for Tₜ-cooling. Tuning these two apparently orthogonal parameters—composition of the ELP segment and the fraction of helical residues in the POP—at the sequence level provides a dial to tune the temperature for the onset of thermal hysteresis and the temperature range of hysteresis at the sequence level. These attributes are likely to be useful for specific applications that require hysteresis to encode memory effects.

### Example 5

### A Model for the Mechanism of Hysteresis

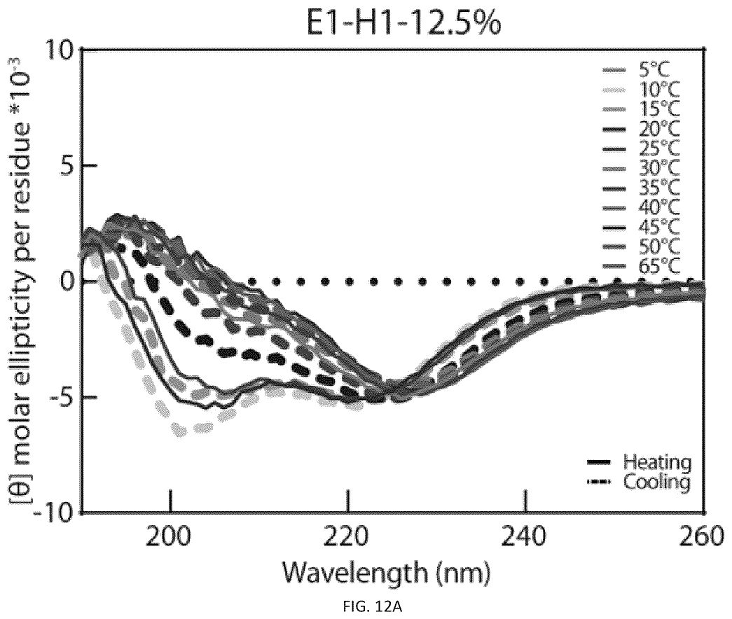

Thermal hysteresis is commonly attributed to changes in secondary structure. Because polyalanine can adopt coil, helical, and beta configurations (Ding, F., et al. Proteins 2003, 53, 220-228), we first analyzed POPs to determine if a secondary structure shift upon aggregation was driving this behavior. UV-CD spectra of a hydrophilic POP (E1-H3-25%) indicate that, in the absence of self-associations, the polymers retain a high degree of helicity up to 65° C. (FIG. 8). POPs that do phase separate show distortions in the UV-CD spectra that are consistent (FIG. 12) with those observed for proposed helical bundles of tropoelastin. These spectral shifts suggest the presence of bundled helices within the POP assemblies. This observation is consistent with the proposed coacervation of tropoelastin, in which polyalanine domains retain their structural integrity during coacervation to stabilize side-chain interactions for crosslinking.

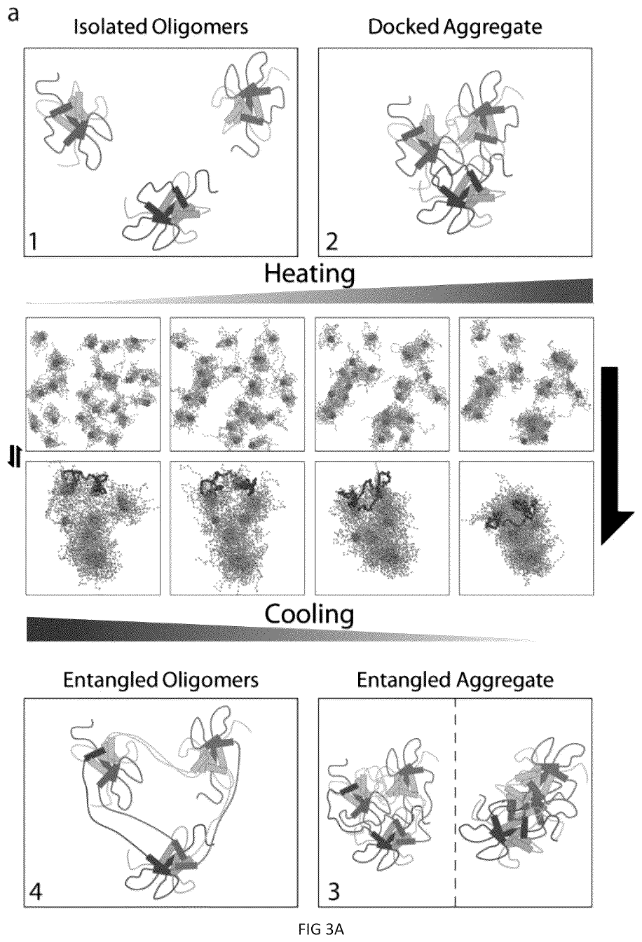

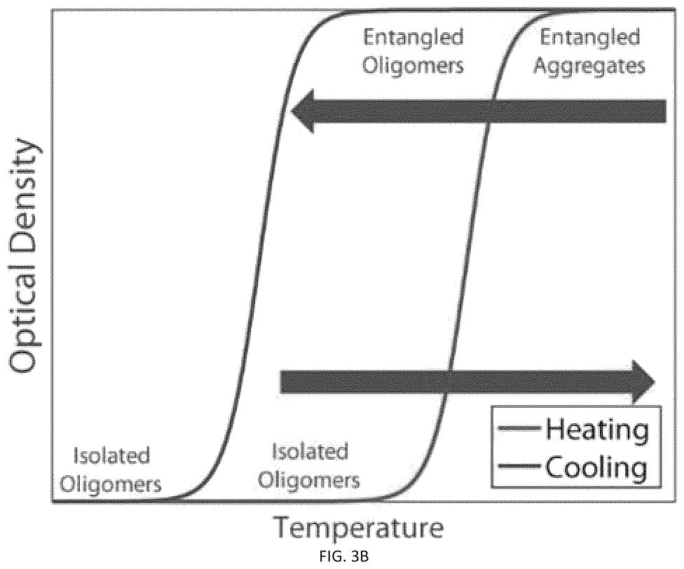

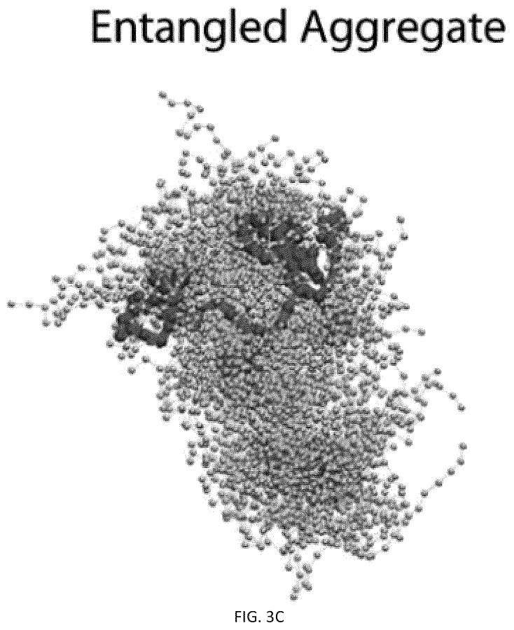

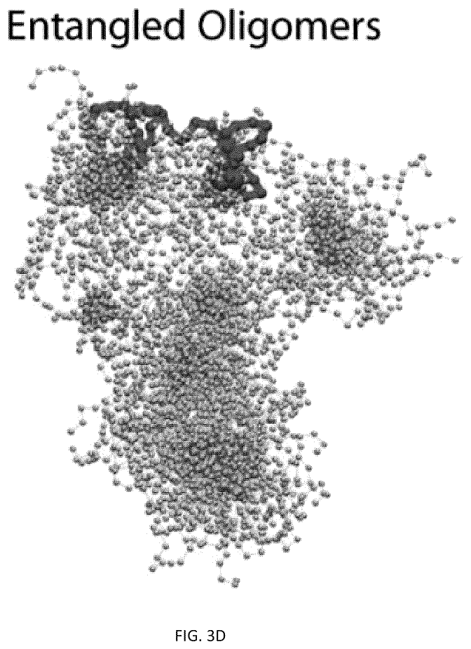

Given the intrinsic tendency of polyalanine to form helical bundles (Bernacki, J. P. & Murphy, R. M. Biochemistry 2011, 50, 9200-9211; Miller, J. S., et al. Journal of the American Chemical Society 2002, 124, 945-962) and the persistence of helices within POP aggregates, we propose helical bundling is a significant contributor to hysteresis. To test this proposal, we performed proof-of-concept assessments using coarse grain molecular dynamics simulations. We used a phenomenological model that separates the protein domains into two categories of pentapeptide “beads”: polyalanine (AAAAA) (SEQ ID NO:38) and ELP (VPGVG) (SEQ ID NO:39). Polyalanine interaction energies (E_{AA}) are always preferred because this promotes polyalanine self-associations; ELP interaction energies (E_{EE}) change with temperature, increasing in strength above the Tₜ-heating. ELP-polyalanine interactions (E_{EA}) were always unfavorable. We simulated a hysteretic cycle for 50 polymers of 25% helicity (E1-H1-25%) in a 25 nm radius spherical box. The results (FIG. 3) suggest that POPs move through four stages during a thermal cycle. (1) Below their Tₜ-heating, POPs are isolated oligomers with local helical clusters that are solvated by ELPs. (2) Above the Tₜ-heating, localized clusters dock due to the increased favorability of ELP hydrophobic interactions. (3) Given sufficient time, the alanine domains exchange with neighboring clusters such that single POPs span multiple clusters, entangling them into a percolated network. Swapping helices between clusters is feasible because of the high density in the docked state and is thermodynamically favored through the entropy of mixing. As the temperature increases further and the ELP repulsive term further decreases, a second reversible transition becomes favored where docked spherical clusters convert into denser, less dynamic linear aggregates. (4) Once cooled below the Tₜ-heating, aggregate entanglement prevents dissolution of the ELP domains, producing entangled oligomers. Unlike the fast and irreversible transition from docked aggregates to entangled aggregates (2-3), transitions between entangled oligomers and isolated oligomers are slow. A sufficient drop in ELP interaction energy (below Tₜ-cooling) leads to eventual solvation of the POPs, diluting the clusters and returning them to their original state.

### Example 6

### Formation of Solid-Like, Fractal Networks

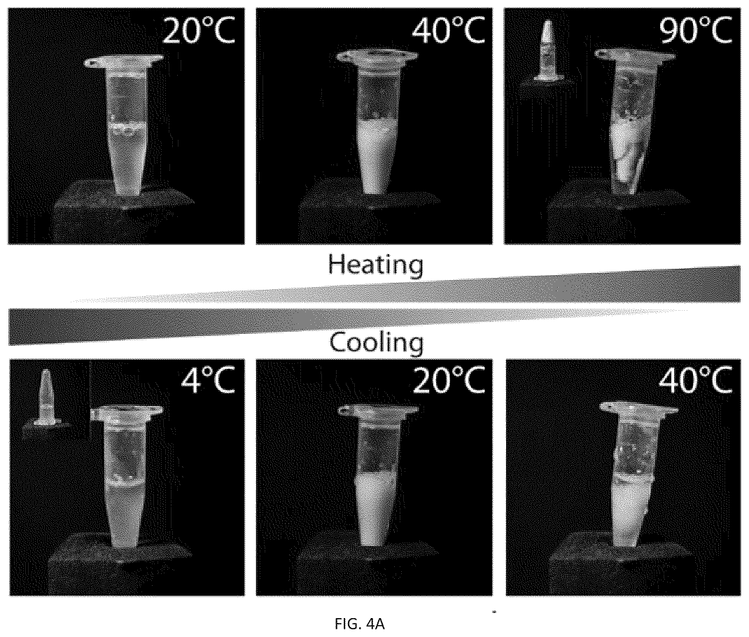

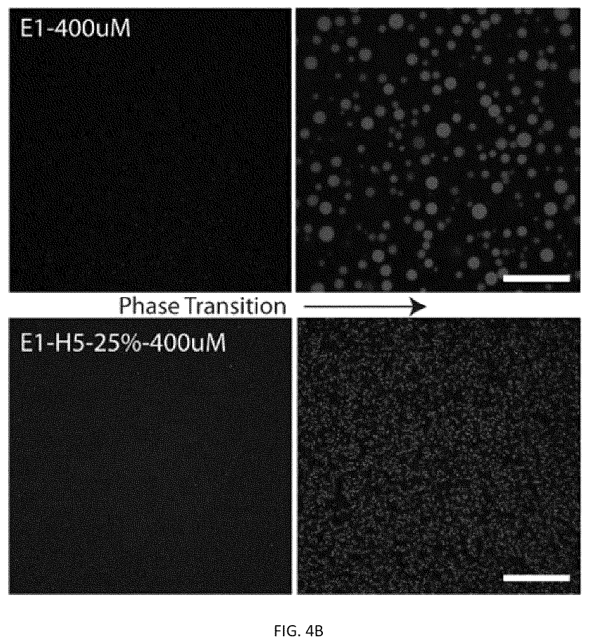

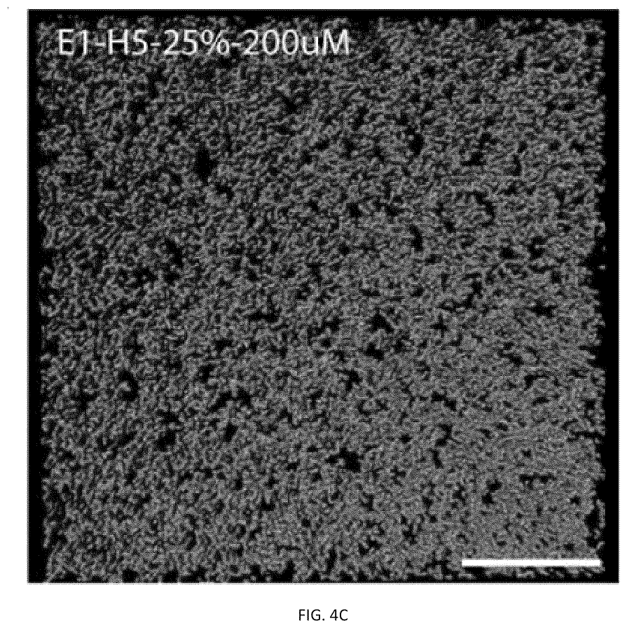

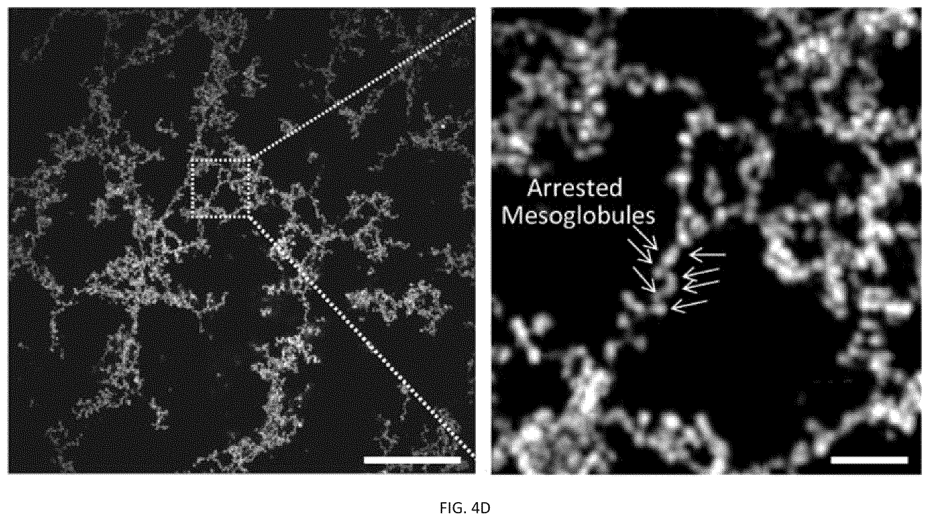

The macroscopic properties of POP aggregates also indicate a mechanism for aggregation distinct from the liquid coacervation of disordered ELPs. Rather than a turbid suspension, POPs transition into mechanically stable, opaque aggregates. These aggregates undergo syneresis at high temperatures, cracking and shrinking as temperatures increase beyond the Tₜ-heating (FIG. 4A). Syneresis suggests percolated crosslinking interactions among polymers, likely due to network formation from the helical clustering that is predicted by our simulations.

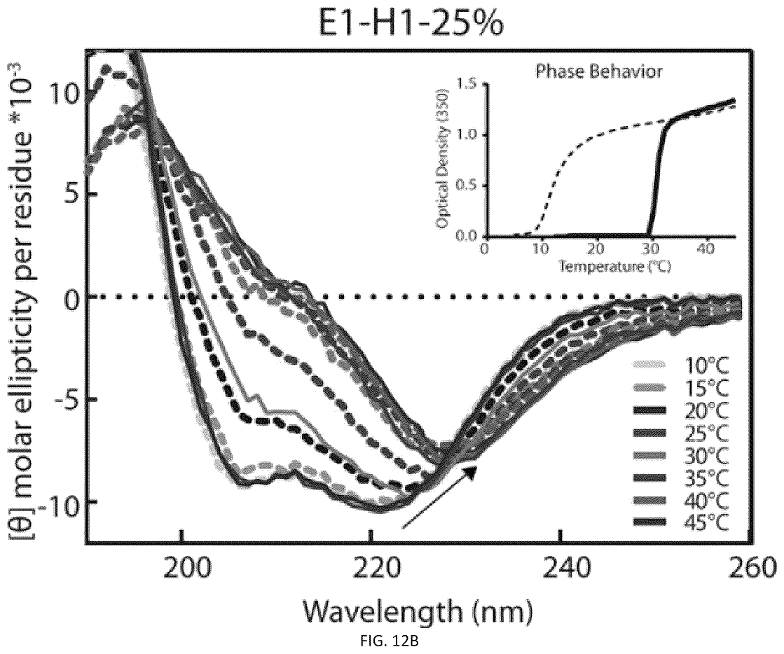

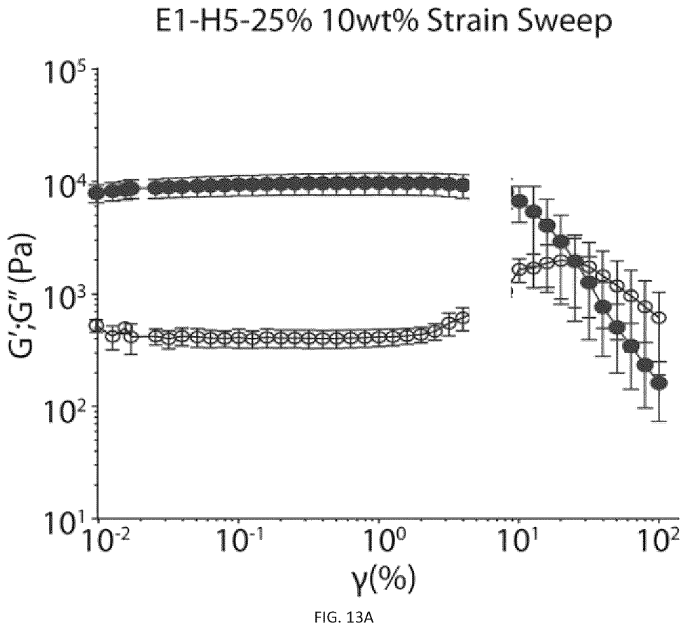

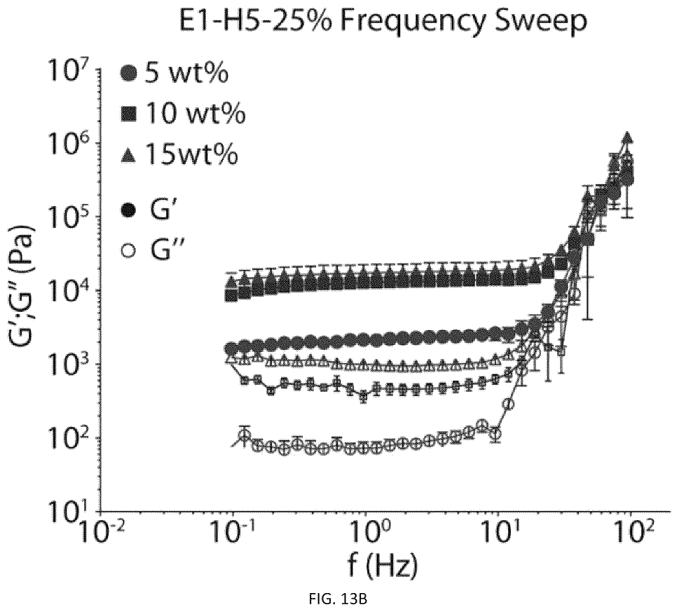

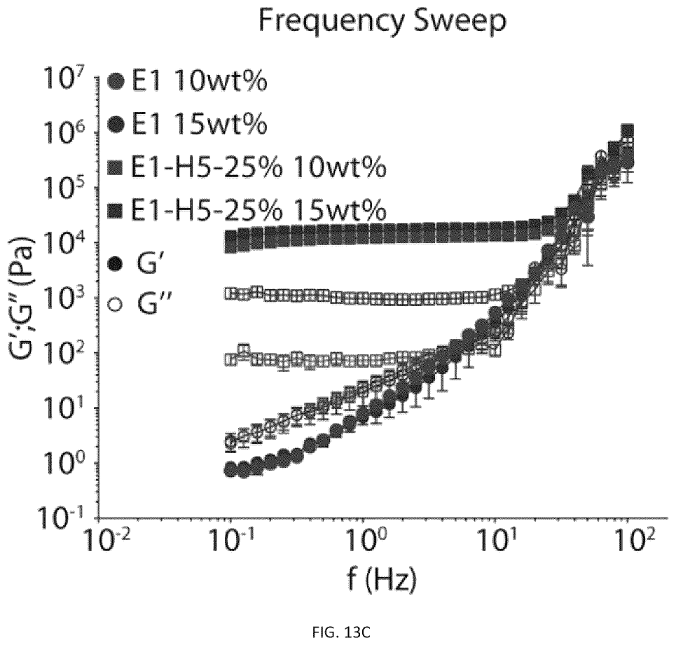

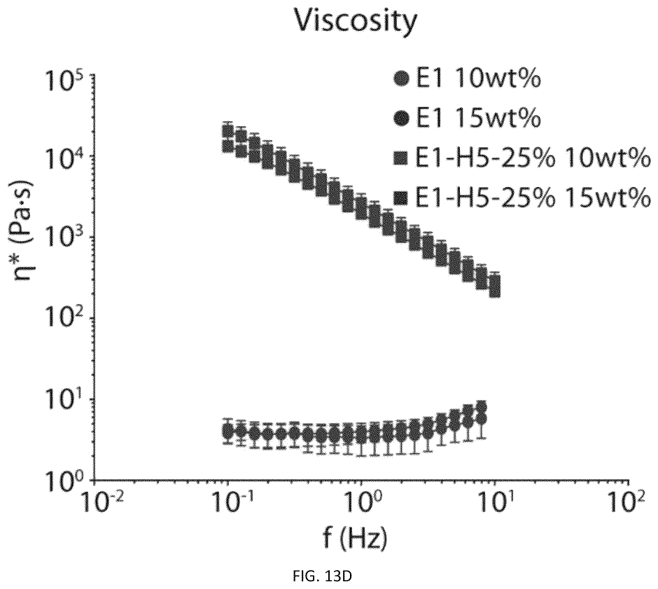

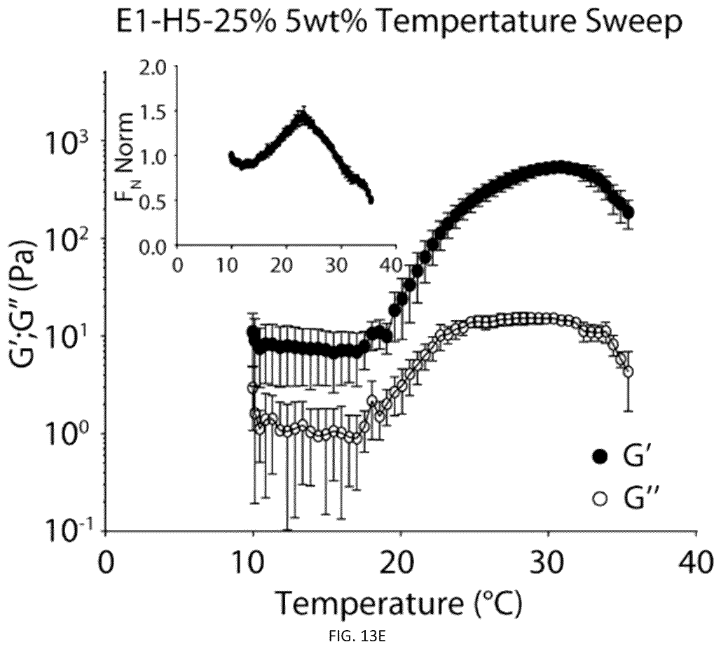

We performed oscillatory rheology on POPs and ELP controls to characterize the mechanical properties of the POP networks and compared them to an ELP control (FIG. 13A-FIG. 13E). Prior to performing oscillatory shear rheology, samples were prepared in PBS and allowed to equilibrate at 4° C. Measurements were taken on a Kinexus Pro (Malvern, Westborough, MA) using a Peltier heating element and a 10 mm parallel plate geometry. Samples were enclosed in a humidified environment to prevent drying during heating, equilibration, and oscillation. Soluble polymer samples were loaded onto the lower portion of the geometry set at 4° C. The upper portion of the geometry was lowered to 0.5 mm, and the instrument was subsequently heated to the experimental temperature (37° C. unless otherwise specified) and allowed to equilibrate for 30 min. To account for volume contraction in ELP and POP gels, samples were run with a normal force control of 0.1N—determined to be the optimal normal force to maintain geometry contact without sample deformation. Uncrosslinked ELP gels were too soft for adequate normal force control, and were instead run with a gap control set to the average gap of their corresponding POP concentration. Each polymer condition was repeated in triplicate. For comparison to chemical crosslinking, an additional ELP (E1₈₀DK) was produced which matched the aspartic acid and lysine distribution of E1-H5-25%, with otherwise identical composition to E1₃₀. Tetrakis(hydroxymethyl)phosphonium chloride (THPC) was used to crosslink available lysines, and, unless otherwise stated, crosslinker was mixed in a 1:1 molar ratio with polymer lysines (a 4:1 molar ratio with polymer). THPC was added to the polymer solution at 4° C. prior heating and equilibration.

Frequency sweeps in the linear viscoelastic region of ELPs above their T, show the loss modulus (G″) (23 Pa, 1 Hz, 10 mg/mL) to be greater than the storage modulus (G′) (8.0 Pa, 1 Hz, 10 mg/mL) and both to be proportional to frequency. This behavior is consistent with liquid-like coacervates. In contrast, POPs exhibit a G′ (12.2 kPa, 1 Hz, 10 mg/mL) that is much greater than G″ (0.36 kPa, 1 Hz, 10 mg/mL) and independent of frequency. This behavior is typical of more solid-like materials. The measured values of G′ were found to be four orders of magnitude greater for POPs when compared to ELPs at equivalent concentrations. POPs also display high viscosity with plastic, shear-thinning flow, while ELPs behave as Newtonian fluids. The shear thinning slope for POPs was unusually high (−0.95) for long-chain, polymers indicative of some network rupture, and this observation is consistent with reported values for tropoelastin networks.

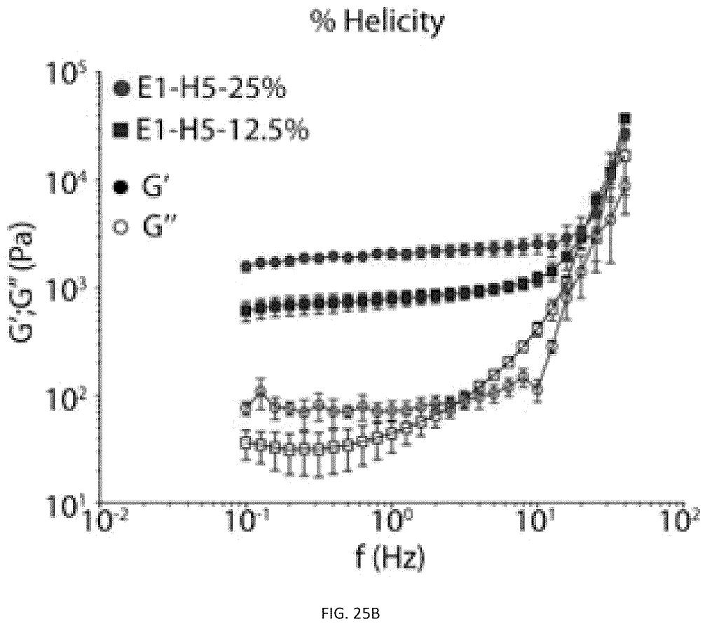

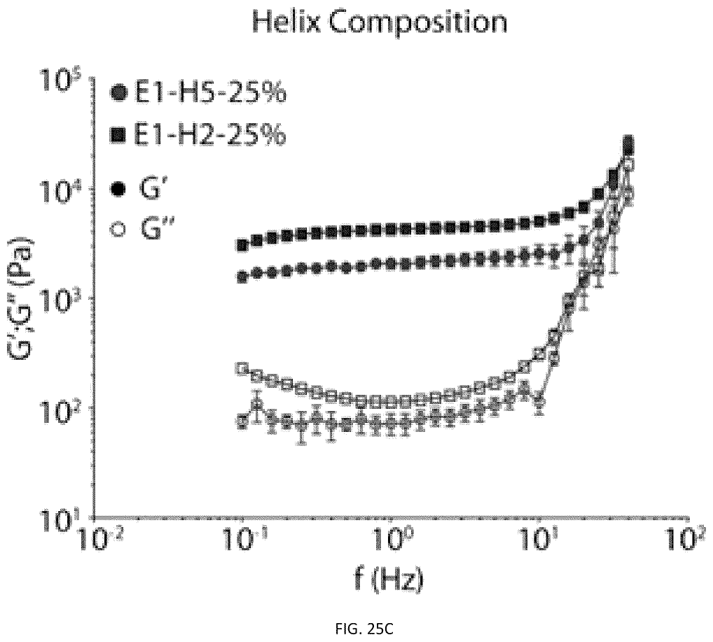

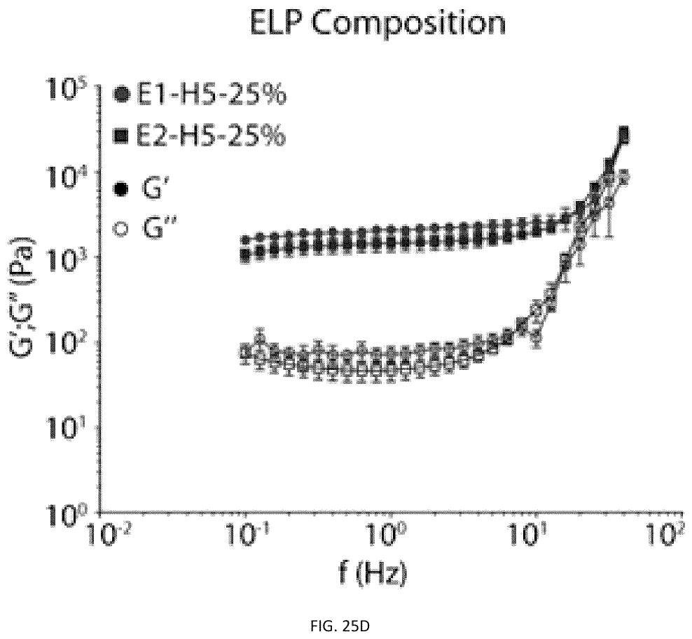

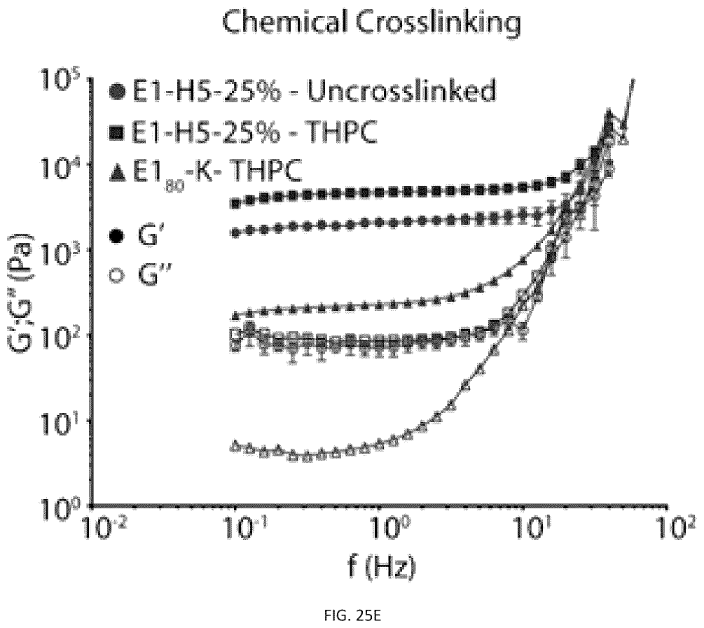

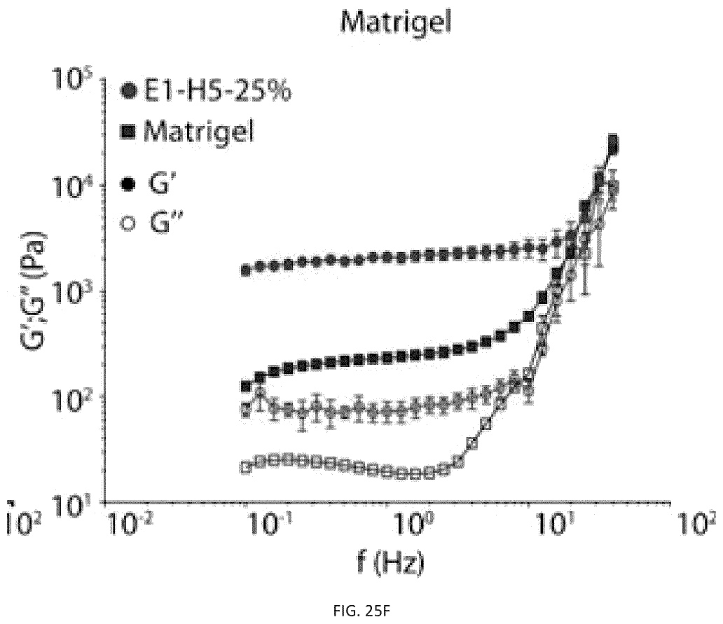

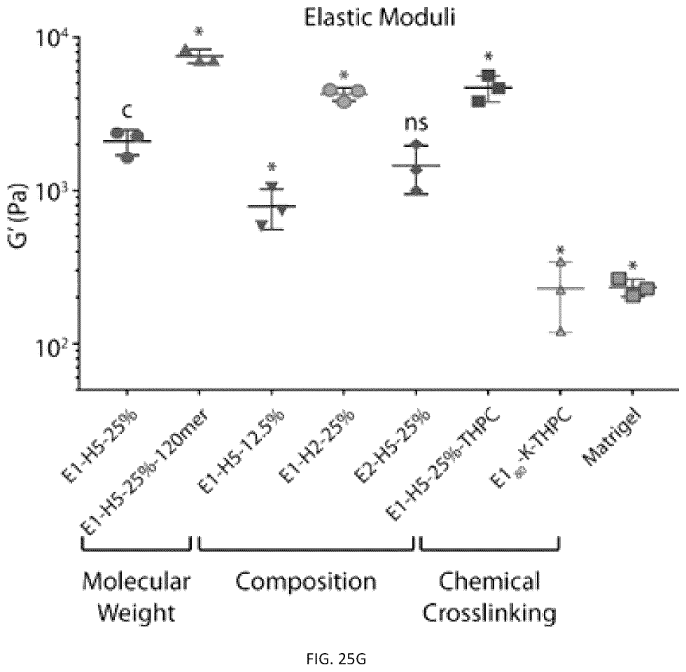

Importantly, POP mechanical properties can be altered with polymer composition (FIG. 25A-FIG. 25G). Material stiffness correlated with MW and helical percentage but is unaffected by the composition of the disordered region. Though POP aggregate stability is driven by physical crosslinking, other crosslinking mechanisms may also be utilized to further modulate mechanical properties. Indeed, POP aggregates chemically crosslinked with Tetrakis(hydroxymethyl)phosphonium chloride (THPC), show a 2-fold increase in stiffness. We also note that chemically crosslinking E1(DK)₈₀, an ELP with identical lysine spacing compared to POP, also increases in stiffness, though their G′ remains an order of magnitude lower than both the chemically crosslinked and POPs lacking chemical crosslinks (FIG. 25A-FIG. 25G). As previous work has shown that fully disordered polymers can achieve similarly high stiffness with sufficient chemical crosslinking densities, even stiffer POPs are likely achievable should the disordered domain be substituted for one with a high density of chemical crosslinking sites.

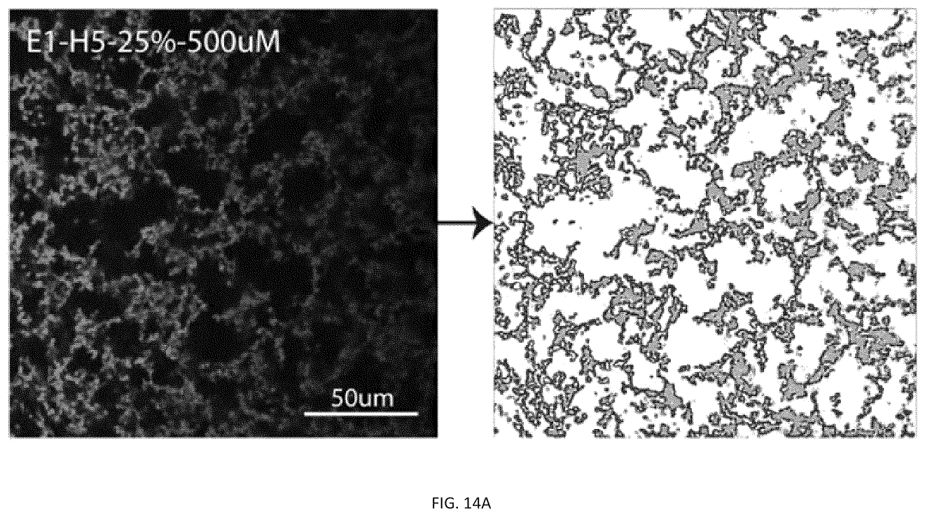

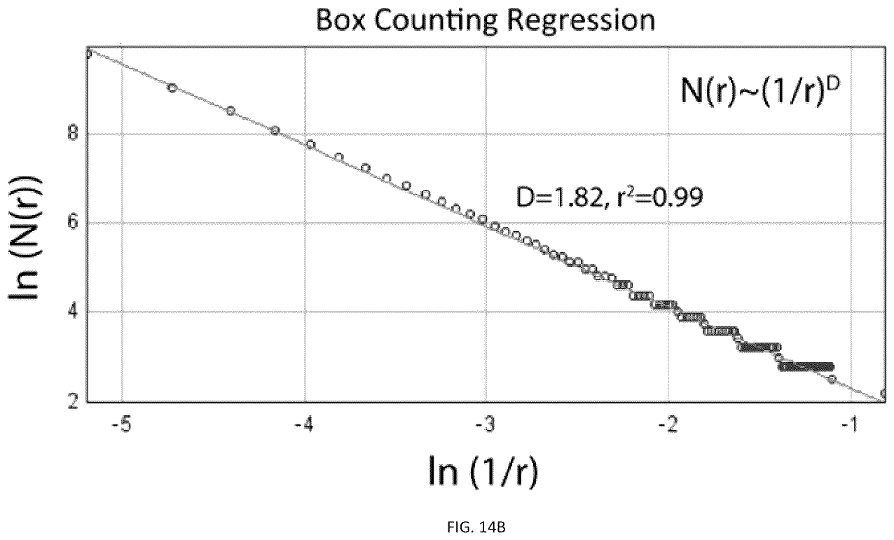

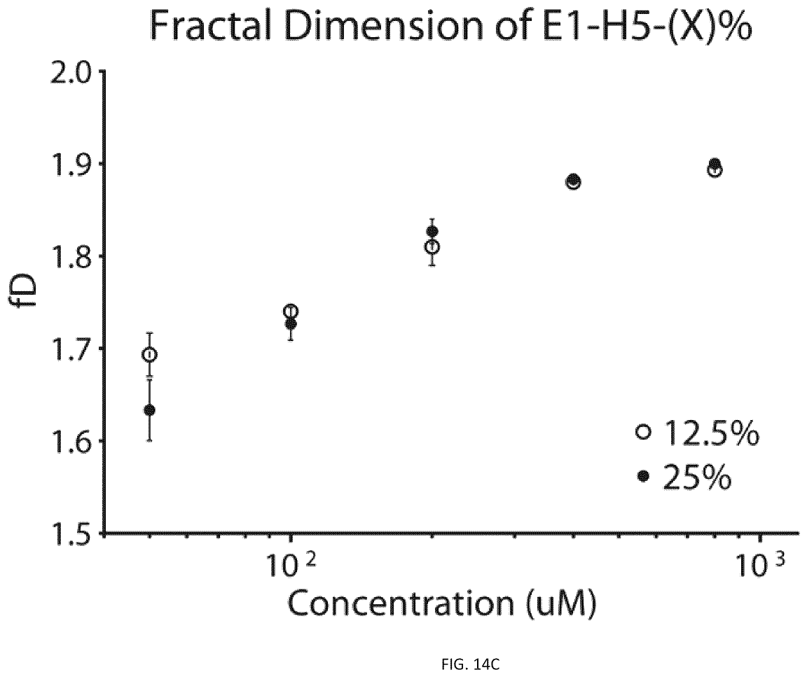

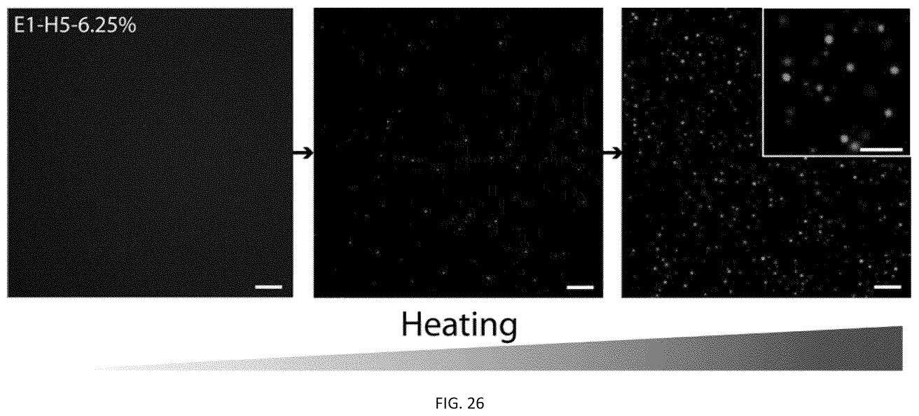

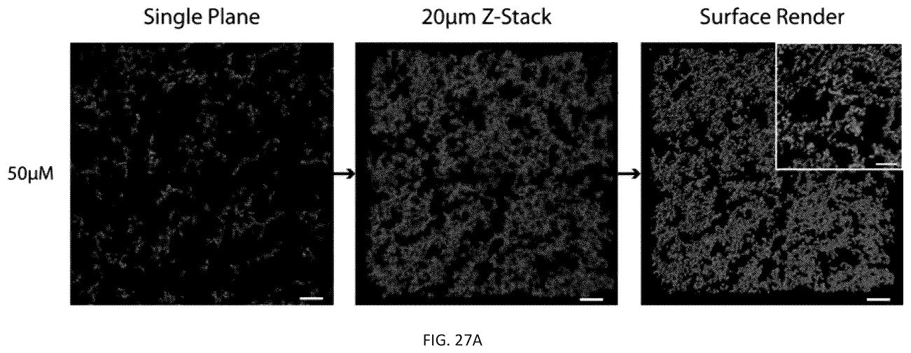

The incorporation of helical domains in POPs also affects microscale phase separation (FIG. 4B-FIG. 4C). While ELPs form micron-sized aggregates that mature and coalesce, forming a colloidal suspension of liquid-like droplets, POPs undergo arrested phase separation into porous networks. POPs with only a single helix form coacervates similar to fully disordered ELPs (FIG. 26), indicating that physical crosslinks between helical domains from separate POP chains are important for network formation. These networks have a fractal-like architecture, with E1-H5-12.5% and E1-H5-25% POP networks having fractal dimensions between 1.6 and 1.9 that are dependent on POP concentration (FIG. 14). This fractal dimension is comparable to that observed for native elastin networks. We highlight the fractal-nature of POP networks as an intriguing observation because fractals are ubiquitous in nature yet difficult to artificially recreate.

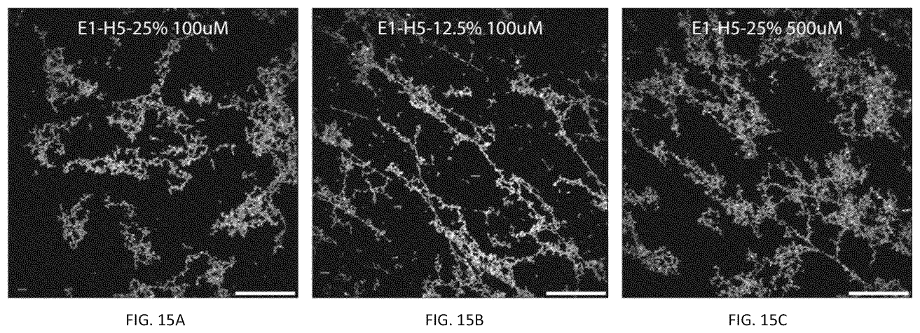

We next used structured illumination microscopy (SIM), a super-resolution microscopy technique (Gustafsson, M. G. L. SHORT COMMUNICATION. Journal of Microscopy 2000, 198, 82-87; Gustafsson, M. G. Proc. Natl. Acad. Sci. U.S.A. 2005, 102, 13081-13086), to better characterize network architecture. SIM revealed the presence of mesoscale polymer globules no larger than 200 nm interconnected with a “pearl-necklace” like architecture (FIG. 4D). This architecture is consistent across multiple polymer compositions (FIG. 15A-FIG. 15C) and is suggestive of a two-stage aggregation process. The polymers initially nucleate like their disordered counterparts (Tₜ-heating driven by the disordered domains). Rather than coalesce, however, the aggregates rapidly link, forming fractal networks. Indeed, our coarse-grain simulations also predict a two-stage process on the nanoscale (aggregate docking and entanglement), and we propose that similar entanglements must also occur on a meso to micro-scale. This type of aggregation is mirrored in tropoelastin, which also undergoes a multistage aggregation process. This process includes an initial hydrophobic coacervation into spherical droplets and subsequent maturation into networks or fibers due to interactions between crosslinking domains.

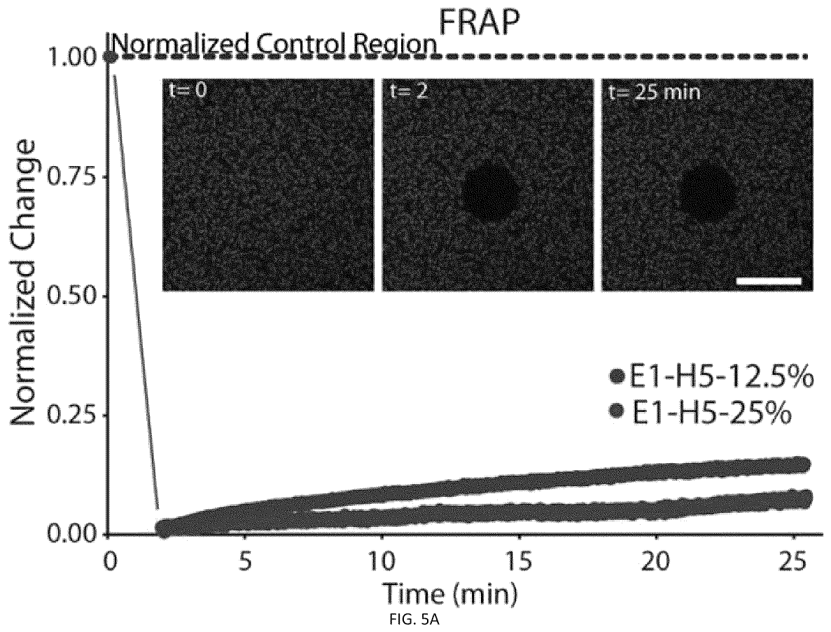

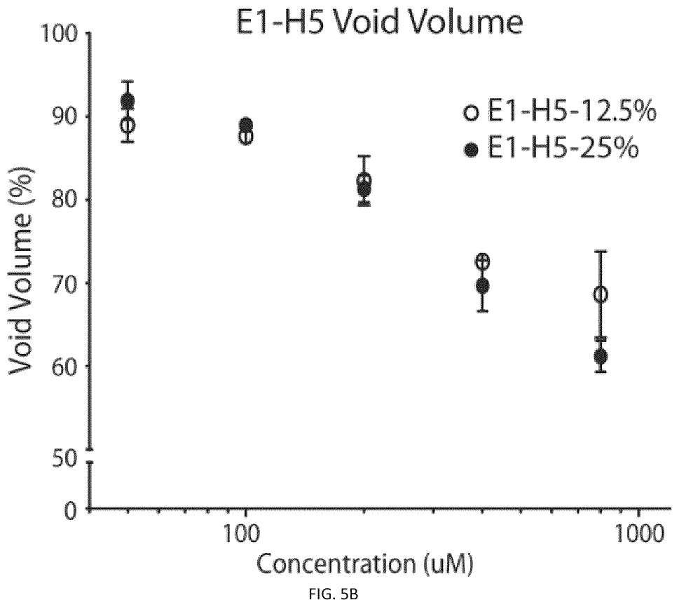

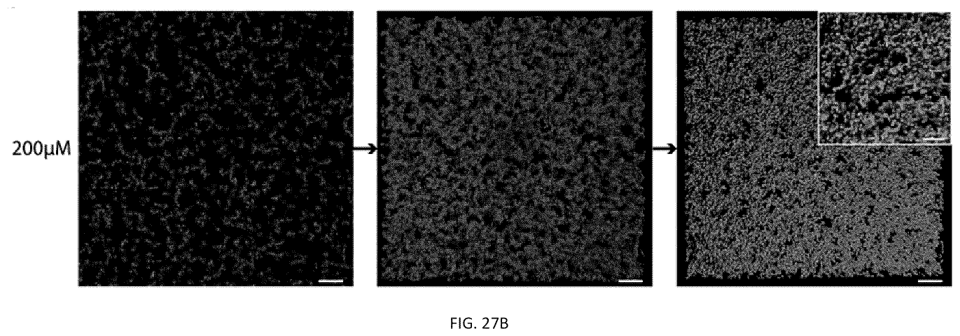

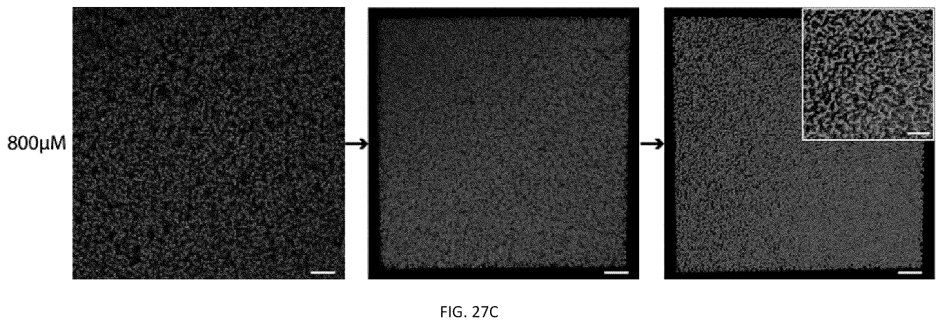

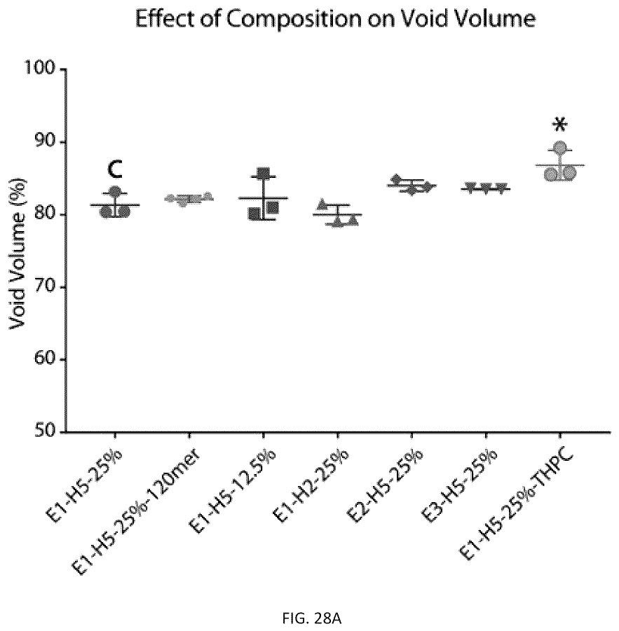

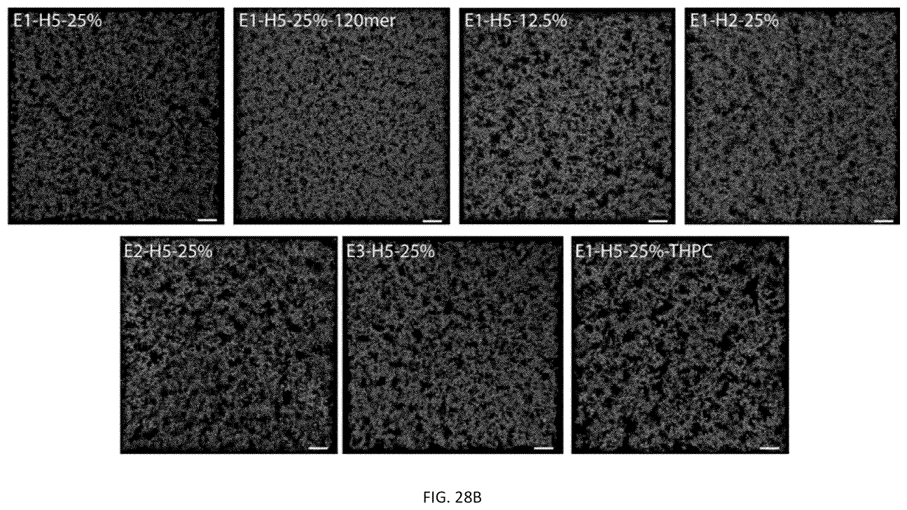

We also measured the internal mobility of POP networks prepared from 12.5 and 25% wt. % POP solutions, by monitoring their fluorescence recovery after photobleaching (FRAP). Minimal recovery was observed after 30 min, suggesting that POP networks are kinetically stable (FIG. 5A). This kinetic stability is likely due to physical crosslinking from helical bundling within the network. There is slightly more recovery for 12.5% networks, but the unrecovered fraction remains high (86%). We can also control network porosity by modulating polymer concentration. Using three-dimensional reconstructions from confocal microscopy, we evaluated the effects of concentration and polymer composition on total void volume, defined as the non-protein rich phase of the network. Within a range of 50 μM (1.6 mg/ml) to 800 μM (25.6 mg/ml) for E1-H5-(X) %, the void volume can be tuned between 90% (˜30-50 μm pores) and 60% (˜3-5 μm pores), with no significant difference in void volume observed between the POPs with 12.5% and 25% helical content over all tested concentrations (FIG. 5B, FIG. 5C and FIG. 27). We also measured the void volume for a variety of POP compositions and found that polymer composition—including changes to MW, helical percentage, helix sequence, and ELP sequence—had no measurable impact on void volume (FIG. 28). This finding allows us to tune porosity of the POP network independently of other network properties. Having porosity as an independently tunable parameter provides a stepwise means to tailor POP networks to specific applications. Toward this end, we envisage the following protocol to orthogonally tune POP properties: (1) choose the desired porosity with concentration; (2) choose other physical properties (mechanical properties or Tₜ-cool) with MW, helix sequence and percentage; (3) finally, choose aggregation temperature (Tₜ-heating) with ELP composition. The ability to tailor POP networks in this manner, and their ability to span 2-3 orders of magnitude in elastic moduli ranging from several hundred Pa to >10 kPa, could be useful, for example, in guiding stem cell differentiation, which are known to be very sensitive to the mechanical properties of the 3-D matrix they are cultured in, but which also require control of the diffusivity of the matrix to enable transport of nutrients and biological signaling factors to cells from the surrounding growth medium.

Our work departs in significant ways from previous studies on block copolymers of bioactive or mechanically active folded protein domains with disordered sequences such as ELPs. Hydrogels and fibers have been produced with ordered segments such as coiled-coils and leucine-zippers to alter their self-assembly; however, these studies have focused on the specific impact of more complicated structural peptides rather than the modular incorporation of ordered versus disordered regions. Likewise, copolymers of disordered domains have produced gels and nanostructures, but these too lack the interplay of order and disorder as a design principle to encode higher order structure. Recombinant combinations of peptide sequences derived from structural proteins such as collagen and silk with elastin are more closely related to this study, although neither the precise and tunable control over thermal hysteresis nor the emergence of an interconnected thermally reversible fractal network architecture has been reported in these studies.

Combinations of order and disorder have also been explored in the field of synthetic polymers. Ratios of atactic (disordered) and isotactic (ordered) polymer blocks have been used as a means to control gelation and thermo-responsive phase transitions. Although synthetic polymers and recombinant peptide polymers each have their own advantages, peptide polymers are attractive for biotechnology and biomedical applications because of their biocompatibility and our ability to design absolute molecular levels through recombinant synthesis. This makes them better suited for tuning material properties via precise—genetically encodable—changes of their amino acid sequence.

### Example 7

### In Situ Network Stability and Cell Penetration

POPs designed to transition below the body temperature (37° C.) are advantageous for forming depots in vivo since they can be handled and injected as liquids, yet rapidly form viscoelastic materials when injected in vivo. Although injectable ELP depots have been similarly used for controlled drug delivery, the homogenous liquid nature of ELP coacervates has limited their applications in tissue engineering. Without chemical crosslinking, ELP coacervates are not mechanically stable enough to support cell growth, and their lack of porosity inhibits cell migration. Research has also shown that porous materials more favorably interact with the immune system, preventing foreign body response and inducing the migration of regenerative immune cells. Polypeptides that exhibit thermally triggered hierarchical self-assembly into stable porous networks can expand the scope of applications for recombinant biomaterials.

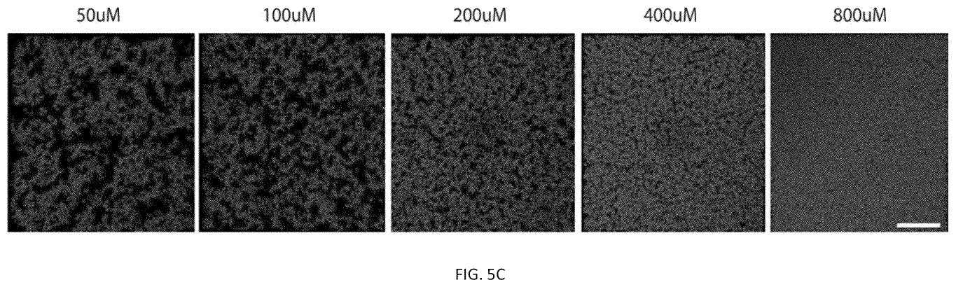

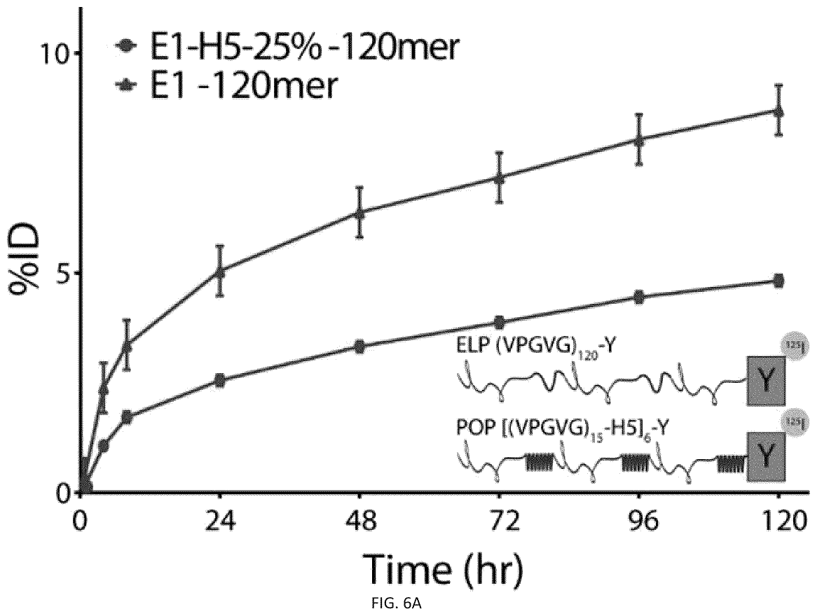

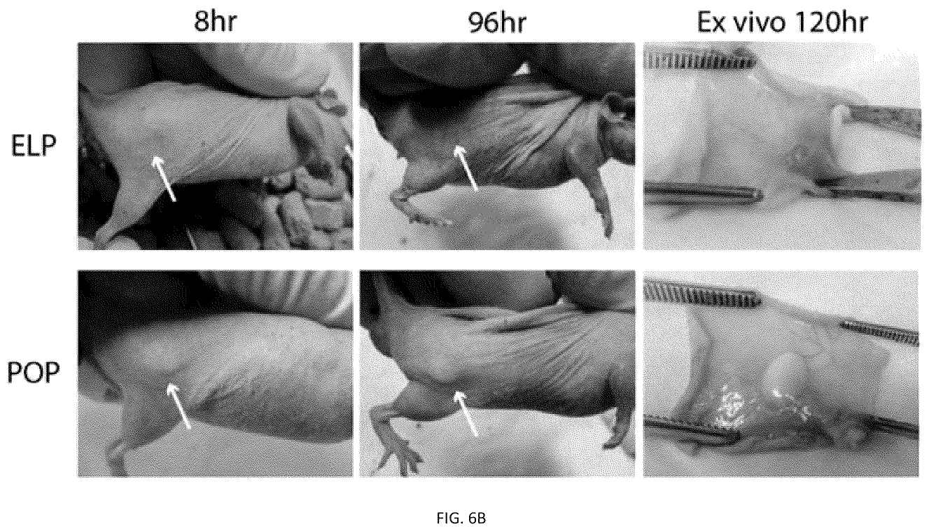

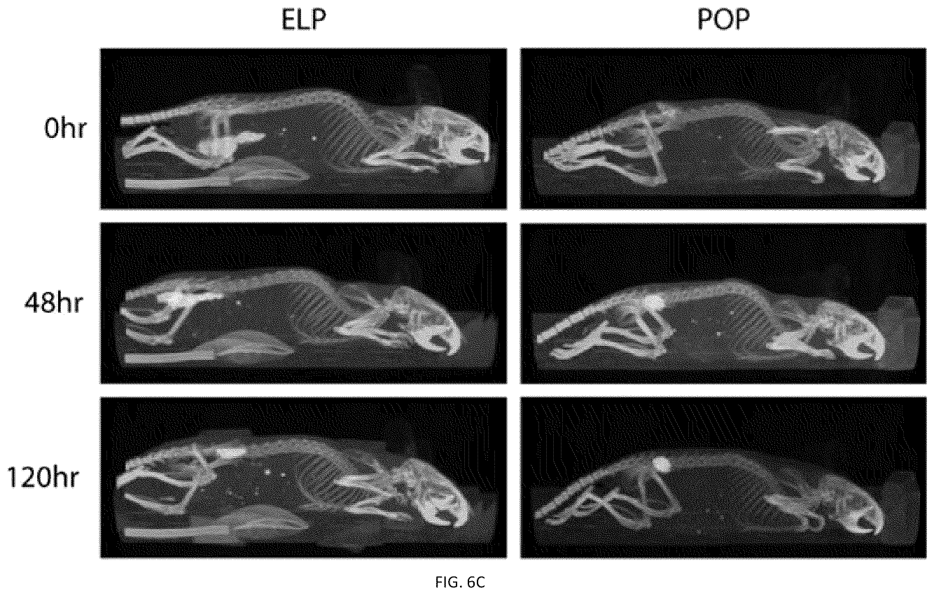

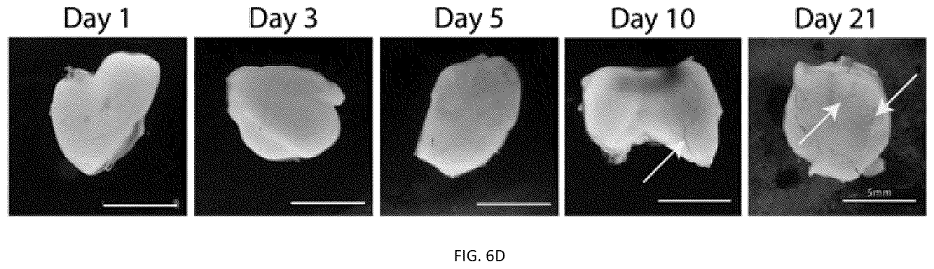

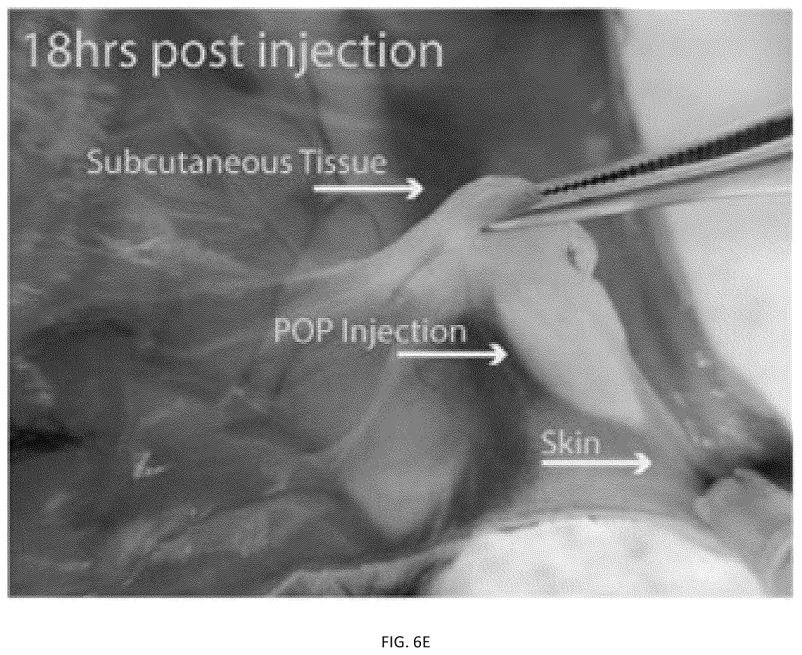

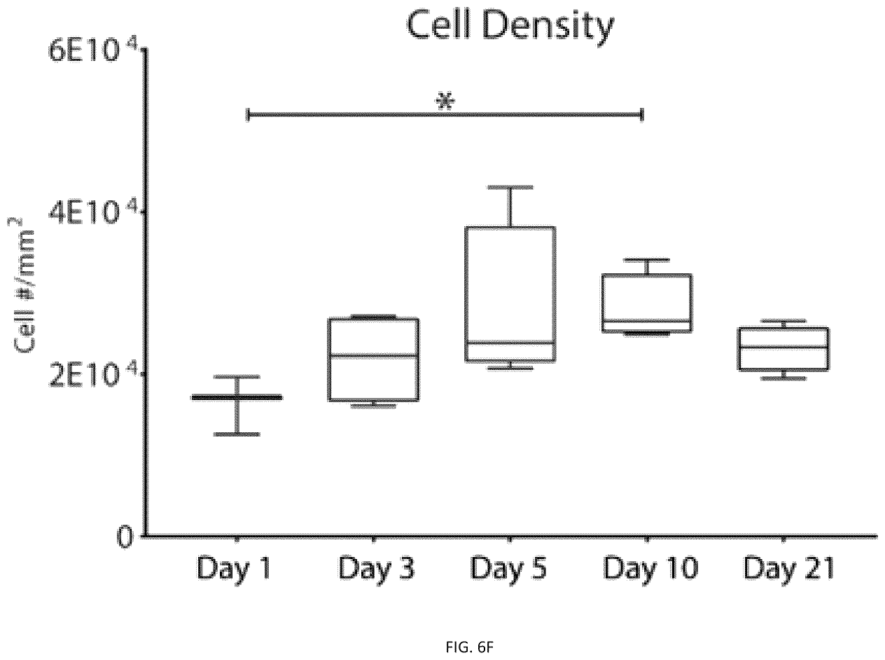

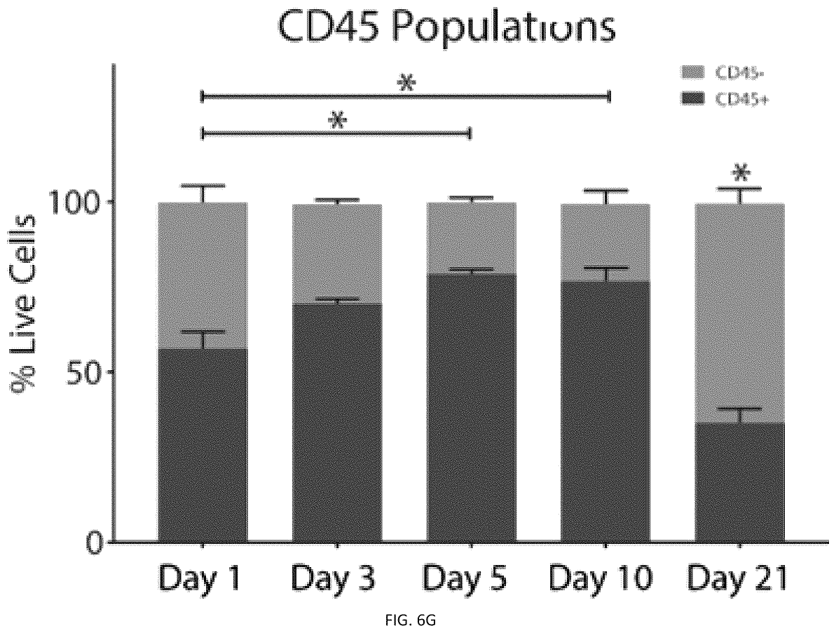

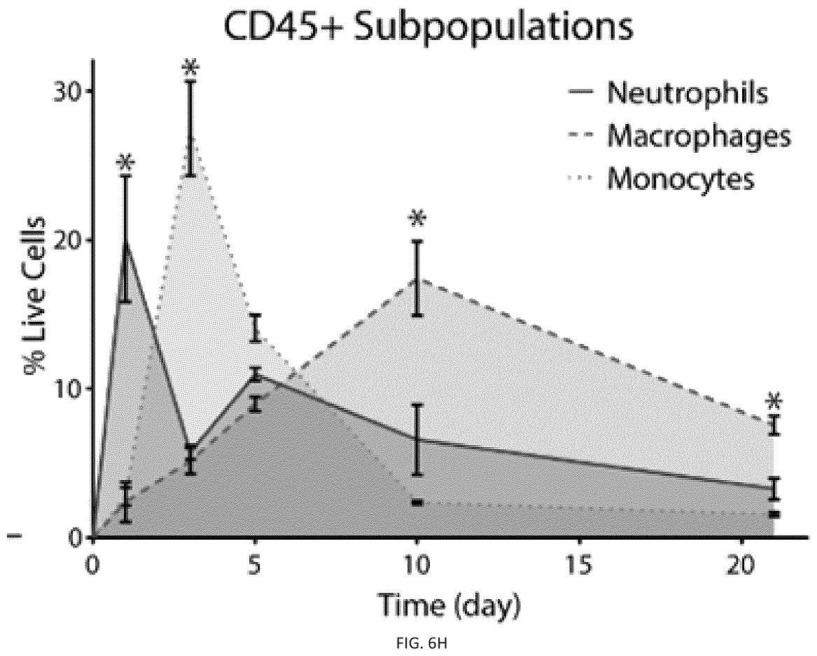

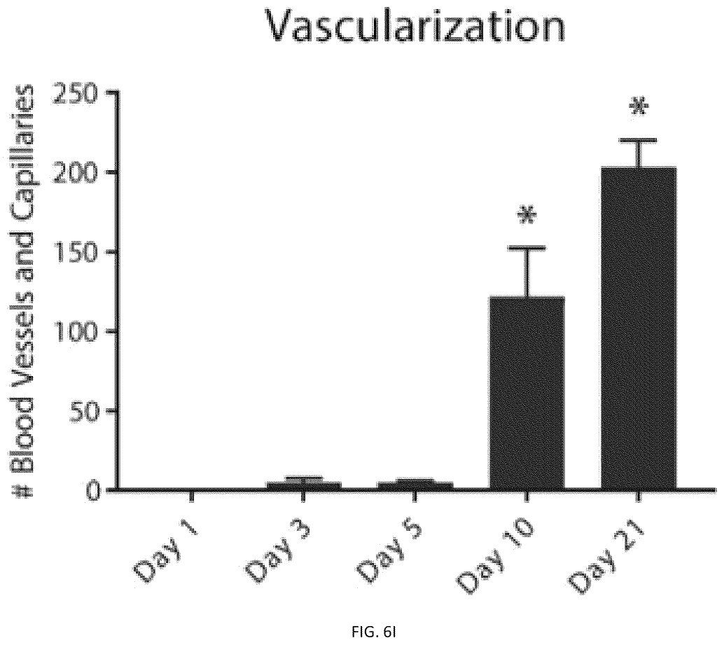

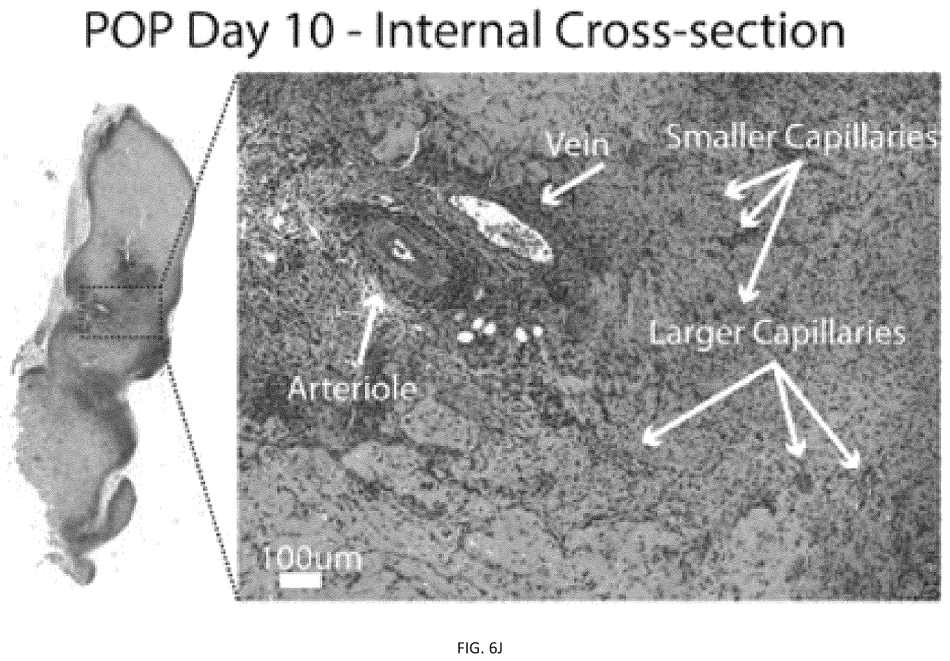

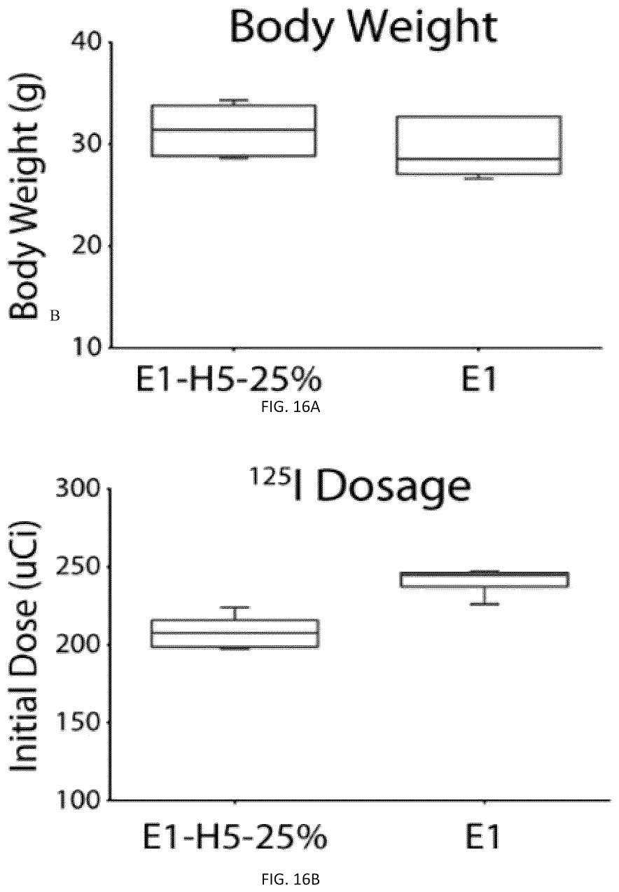

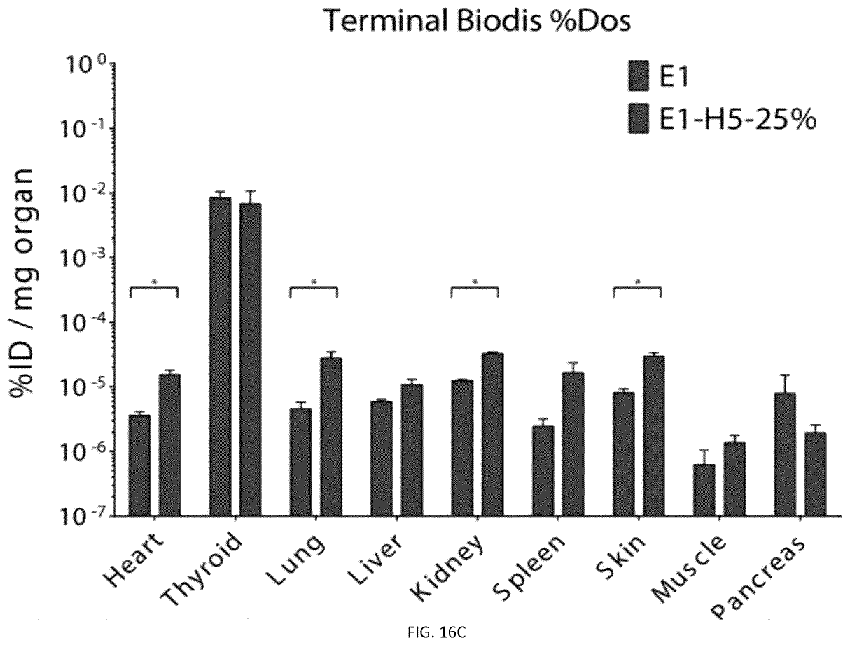

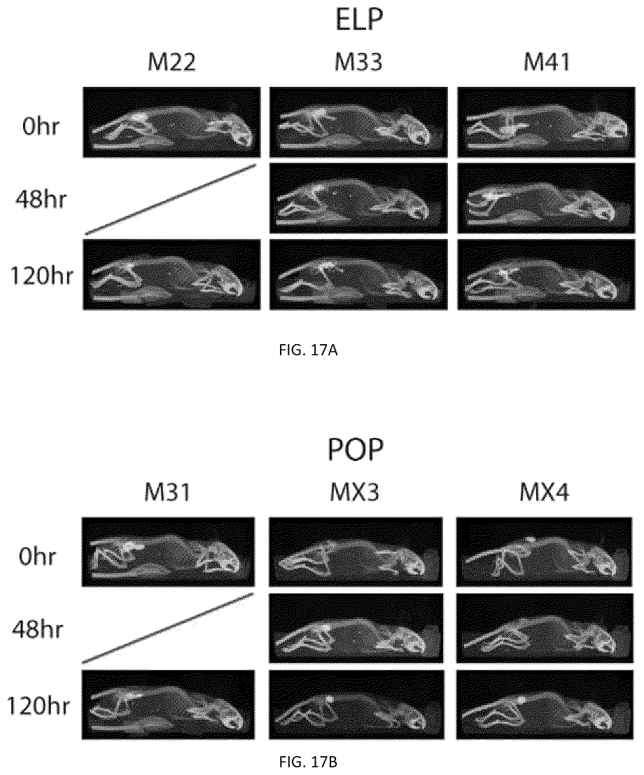

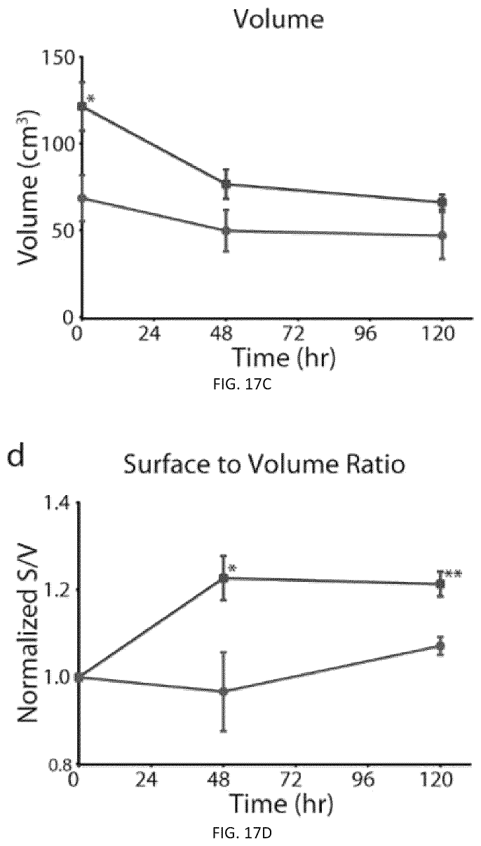

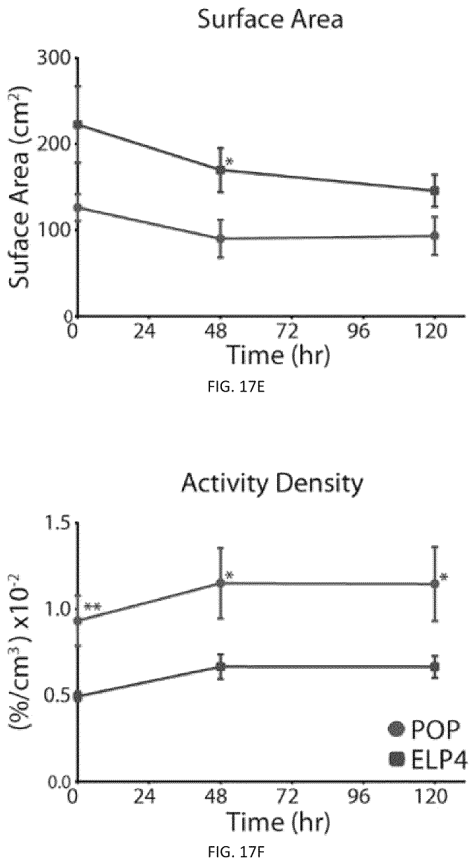

To assess the in vivo behavior of injected POP networks, we injected E1-H5-25%-120 (200 μL at 250 μM, 50 kDa) as subcutaneous (s.c.) depots and compared their pharmacokinetic (PK) properties to fully disordered ELPs of the same base sequence. POP depots, labeled with ¹²⁵I, showed significantly less polymer release (4.8% of initial dose) than their pure ELP counterparts (8.7%) after 120 hours despite their increased porosity and greater surface area (FIG. 6A). Terminal bio-distribution also revealed no critical accumulation in vital organs (FIG. 16). Upon injection, ELPs diffuse in the s.c. space until they are not externally apparent whereas POPs form large, depots that are easily visible through the skin (FIG. 6B). Single-photon emission computed tomography (SPECT) confirms that ELP depots are more diffuse than POP depots with higher surface-to-volume ratios and lower polymer densities (FIG. 6C and FIG. 17).

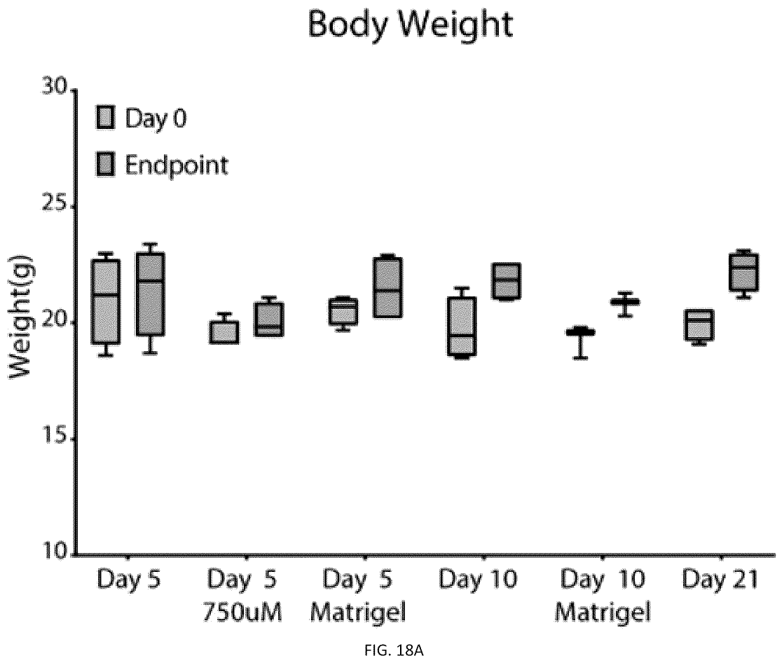

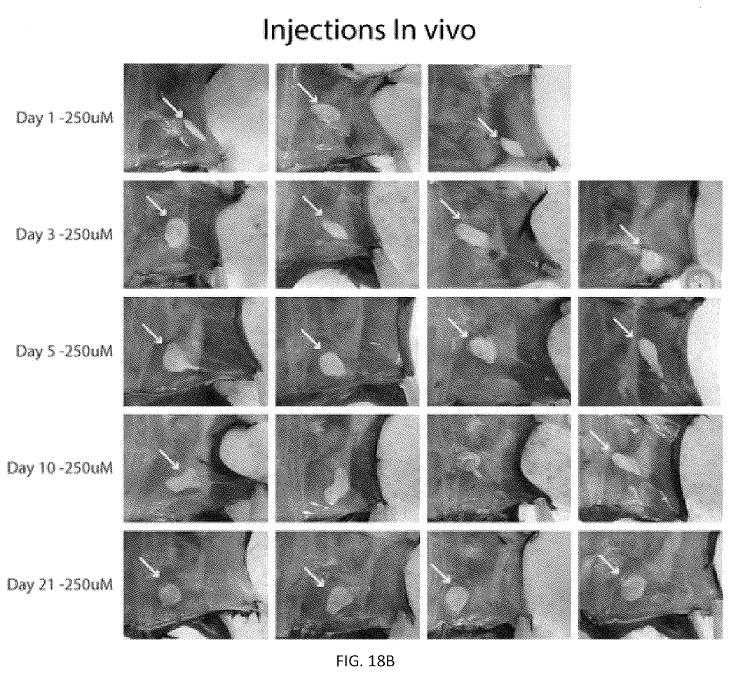

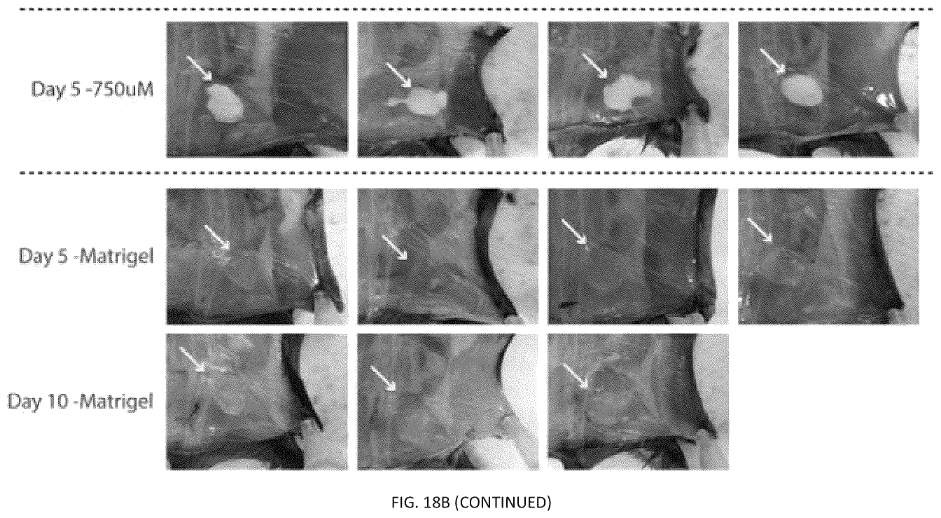

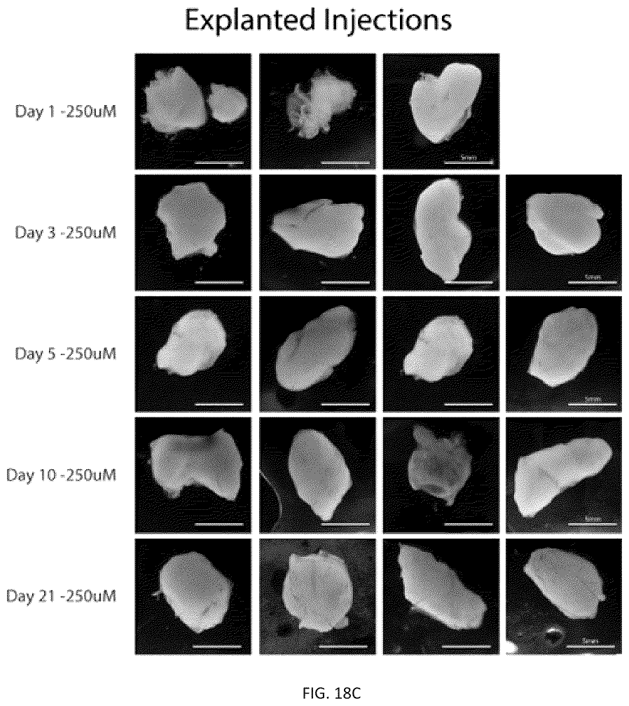

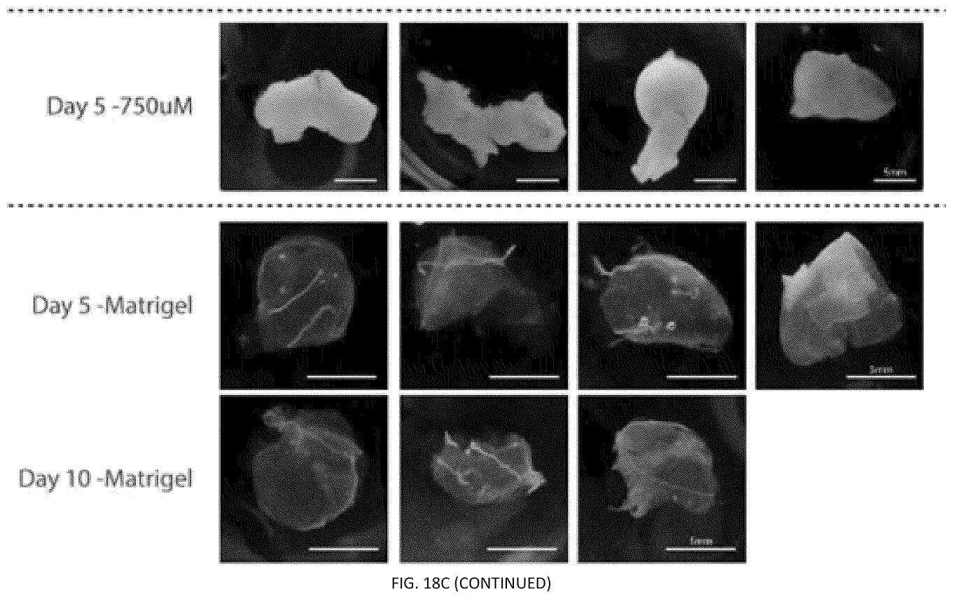

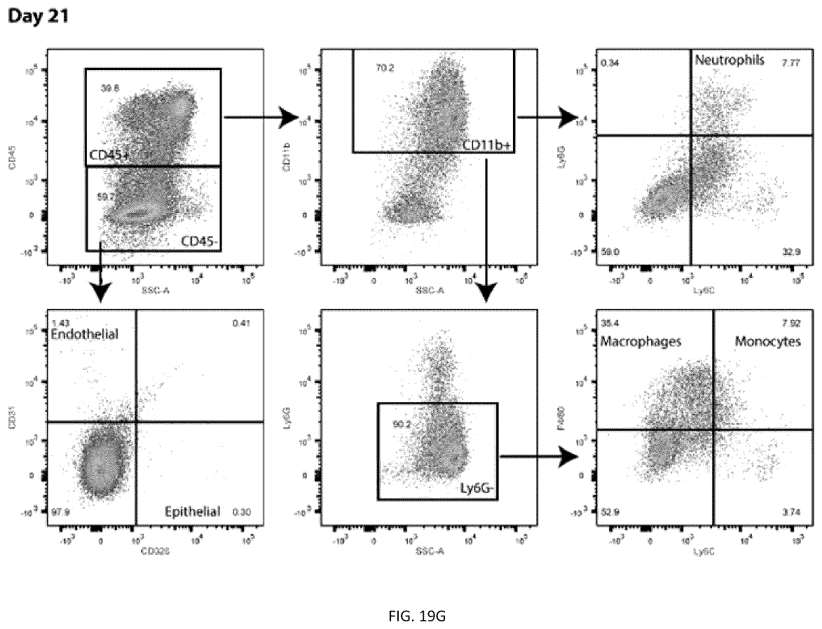

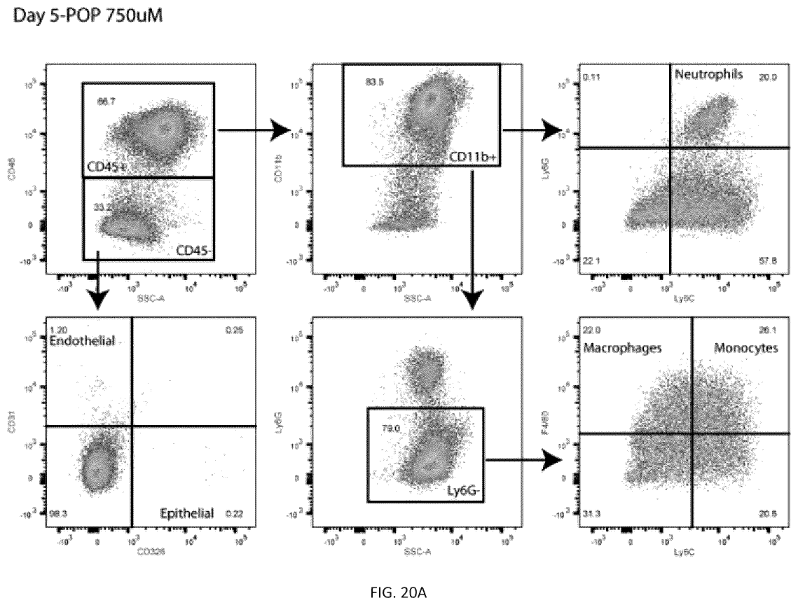

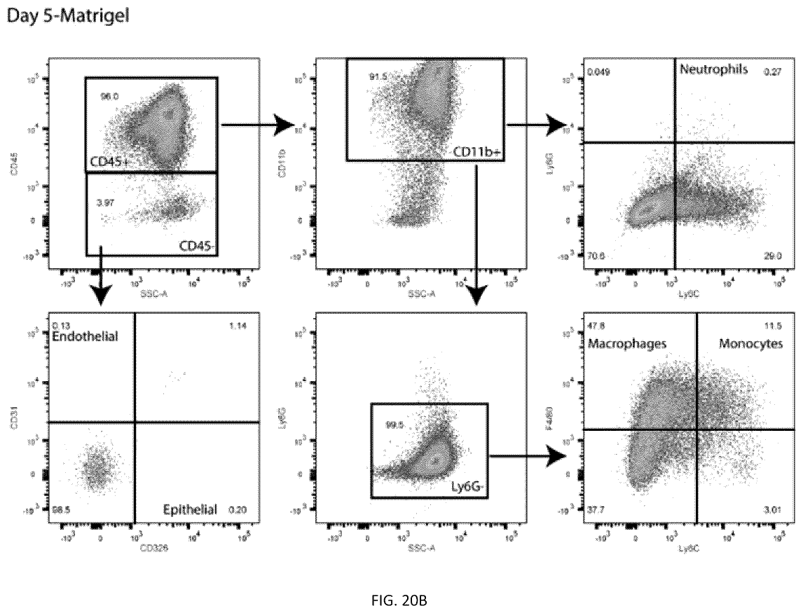

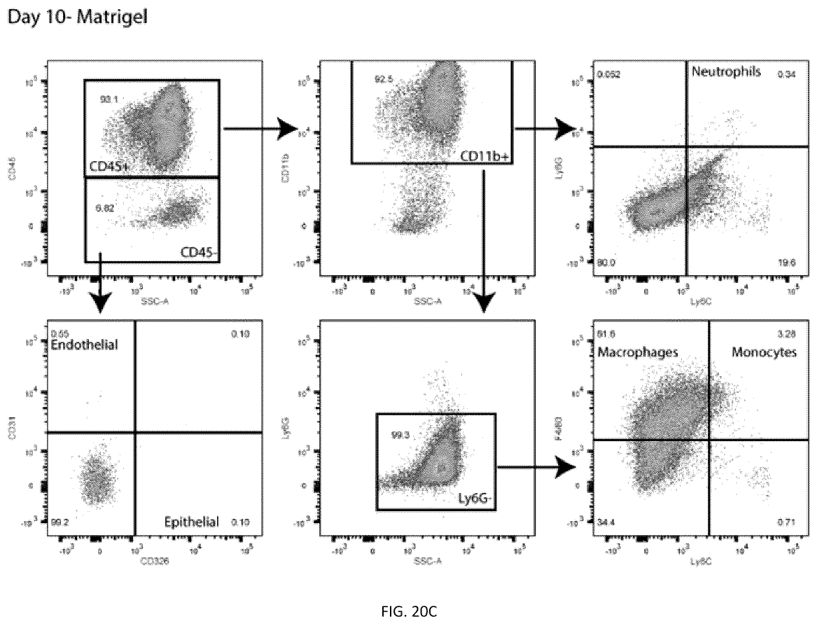

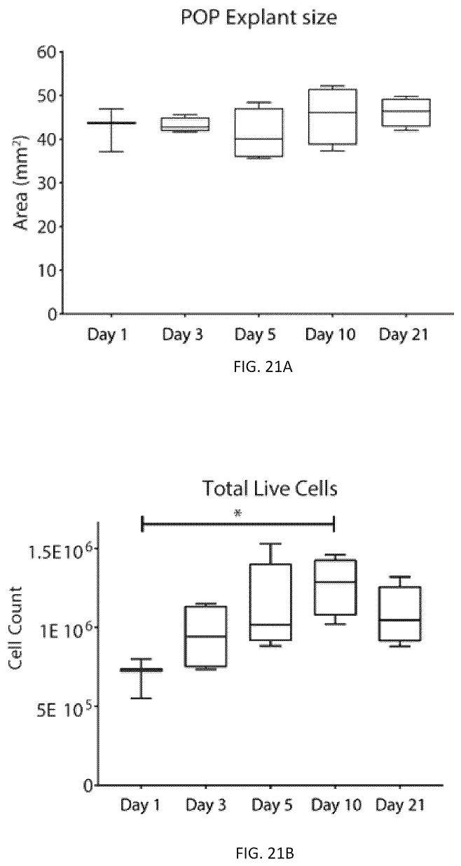

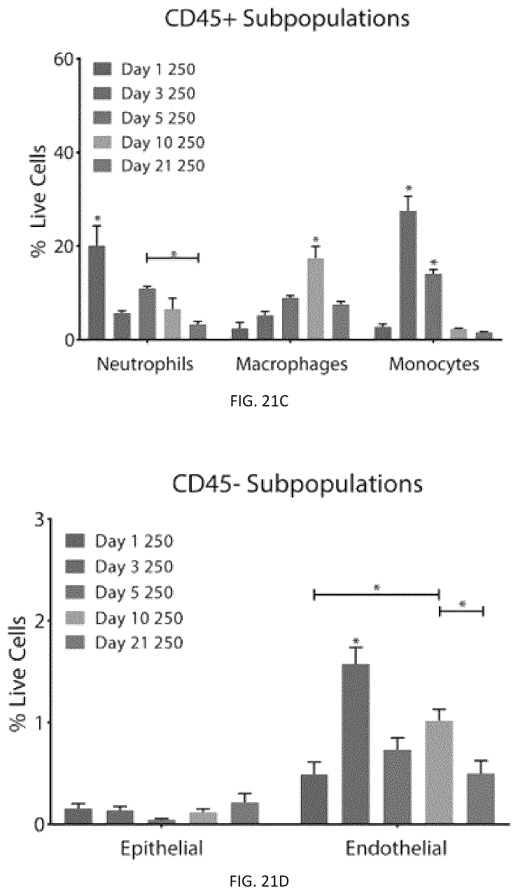

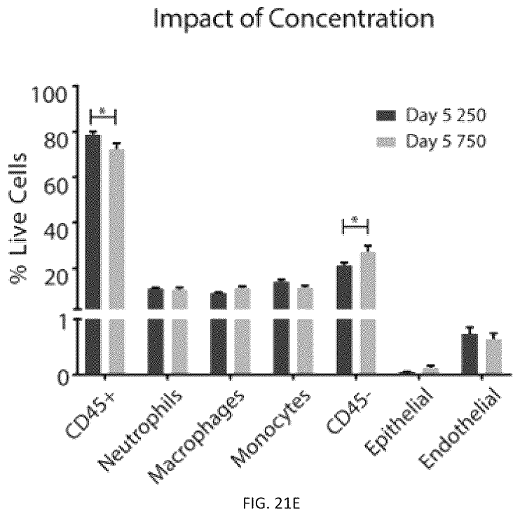

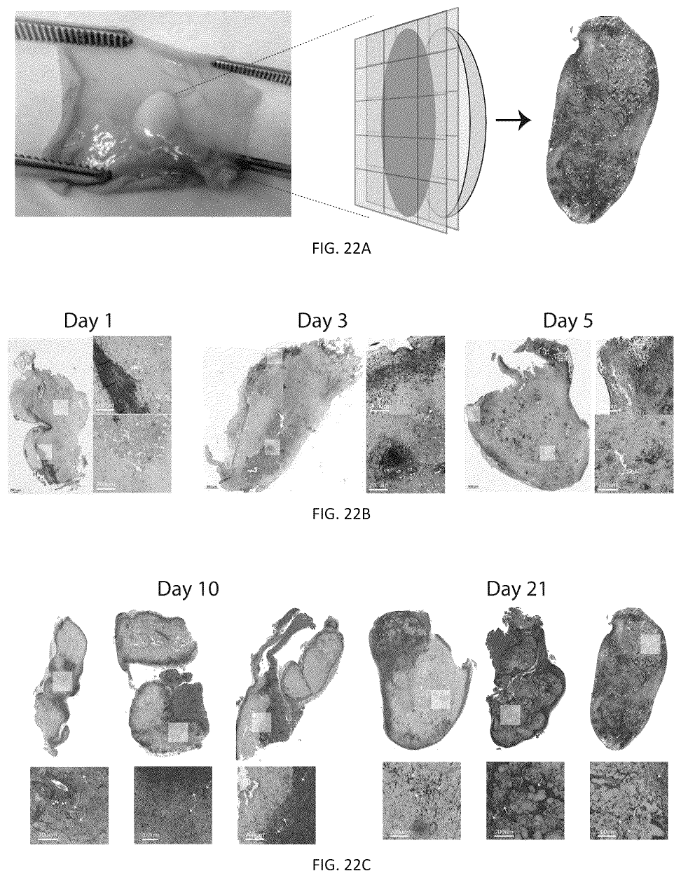

For analysis of POP persistence in the s.c. space and cell recruitment, C57BL/6 mice receiving endotoxin purified (<1 EU/mL) s.c. injections of E1-H5-25%-120 (200 μL at 250 μM, 50 kDa) were monitored over 21 days. Injected depots were excised and either fixed for histological evaluation or processed to extract cells for flow cytometry (FIG. 18-FIG. 20). POPs rapidly and robustly integrate into the s.c. space, creating mechanical connections with surrounding tissue within 24 h (FIG. 6E) and show no significant decrease in size after 21 days (FIG. 6D, FIG. 18, FIG. 15). Initial cell recruitment is high, with cell density peaking at day 10 (FIG. 6F). Recruited cells show the POP depots to undergo a wound-healing response with an initial, mild inflammatory phase that resolves over time followed by angiogenesis and proliferation of non-immune cells. Hematopoietic-derived cells (CD45+) steadily increase up to day 10 with neutrophils, inflammatory monocytes, and macrophages peaking on days 1, 3, and 10 respectively (FIG. 6G-FIG. 6H). By day 21, all hematopoietic derived cells drop off dramatically and non-hematopoietic cells become the dominant population (FIG. 6G-FIG. 6H and FIG. 21). Curiously, E1-H5-25%-120 injected at 750 μM POP concentration did not show significant differences in any recruited cell subtypes from 250 μM (FIG. 21) despite the decrease in porosity. Histology of POP depots supports the presence of a high cellular density, extensive cellular infiltration from surrounding tissue, and no strong fibrin capsule formation (FIG. 22). POPs also show a high degree of vascularization with capillaries and some larger vessels emerging by day 10-with some branching vessels even visible to the unaided eye (FIG. 6D and FIG. 6I-FIG. 6J). The vasculature becomes more uniformly distributed throughout the depots by day 21 (FIG. 22).

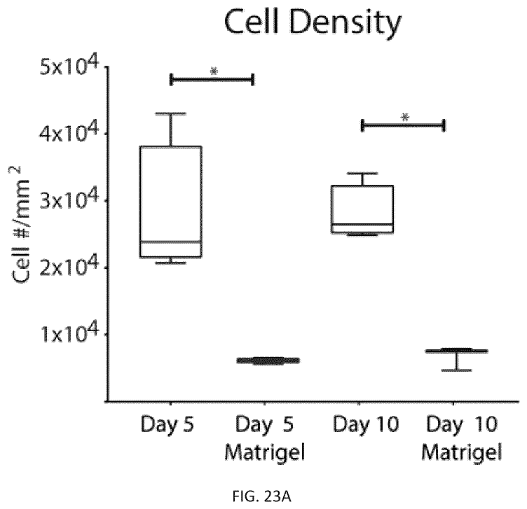

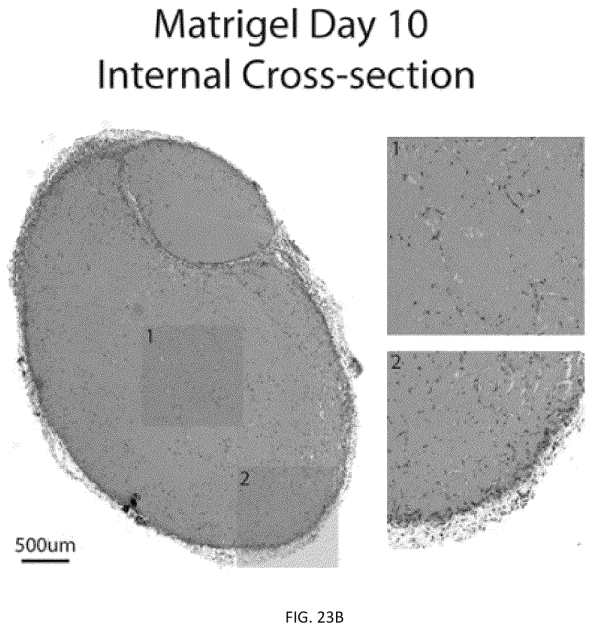

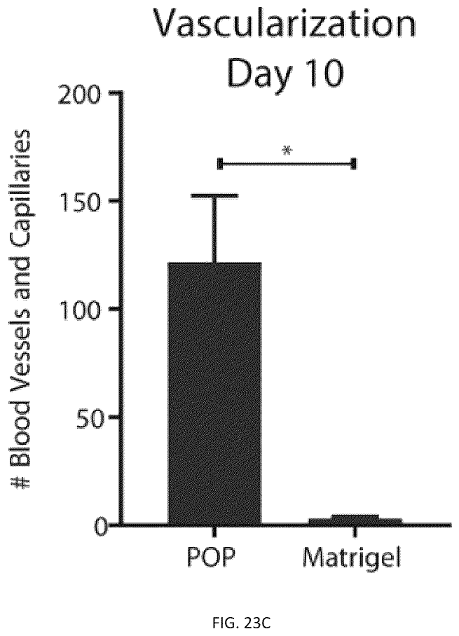

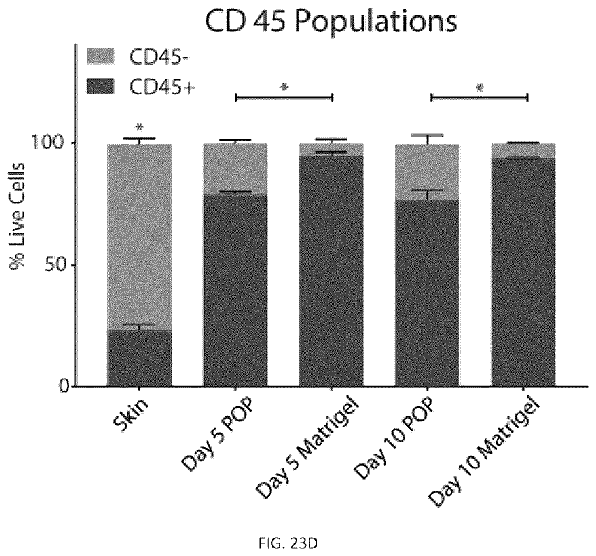

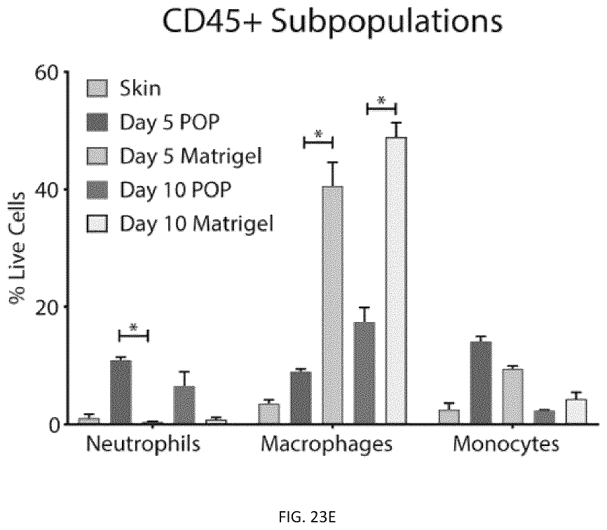

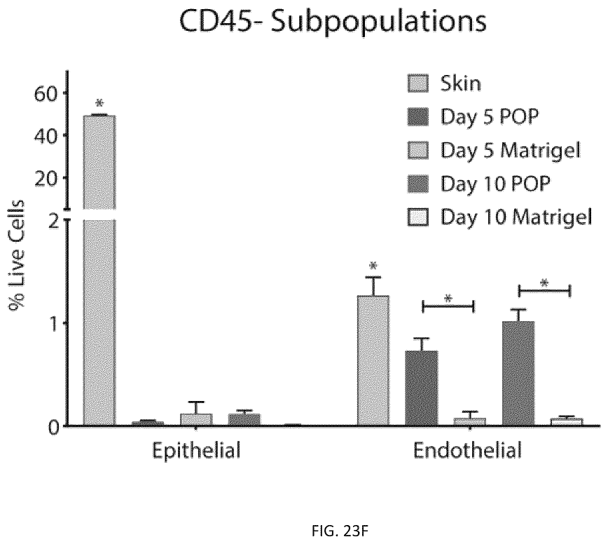

Because fully disordered ELPs disseminate too quickly to form explantable depots, we injected equivalent weight percentages of Matrigel to provide a comparison to an established injectable scaffold. Compared to Matrigel, POPs recruit a greater number of cells, including non-hematopoietic cells, and show dramatically increased mechanical integration and vascularization than Matrigel (FIG. 23). POPs are therefore more useful than Matrigel for applications requiring increased integration of the scaffold surrounding tissue, whereas Matrigel may be more useful for applications requiring greater isolation of the material from surrounding tissue. The angiogenesis of POPs with minimal, resolving inflammation is promising for the use of POPs as an injectable material for regenerative medicine.

Using molecularly engineered polypeptides that precisely encode ordered and disordered segments along the polymer chain, we have developed a simple, modular, and tunable material system to evaluate the impact of molecular order and disorder at the primary sequence level on the structure and properties of the resulting material. By encoding helical domains into ELPs, we show that thermally triggered phase separation does not lead to dense coacervates, but instead drives the hierarchical assembly of porous, viscoelastic networks that are reminiscent of cross-linked elastin. Though the physically cross-linked POP networks retain the thermal reversibility of fully disordered ELPs, the aggregation and dissolution temperatures can be independently controlled by specifying the composition and mass fraction of the disordered and ordered domains respectively. These polymers assemble into 3D scaffolds in vivo that are notably more stable than controls, which are disordered ELP sequences. Analysis of explanted POP depots reveals a progression from mild inflammation that resolves with time, to migration of cells within the scaffold, followed by proliferation and vascularization, indicating that POPs promote wound healing and tissue growth. As the field of intrinsically disordered proteins has expanded, knowledge of the biological importance of the synergy between disordered regions and ordered domains is growing; yet limited information exists on functionalizing these interactions for biomedical applications. Our biopolymer platform is an important step towards uncovering design rules that combine order and disorder to develop a new generation of functional protein biomaterials.

### Example 8

### UV Crosslinkable POPs

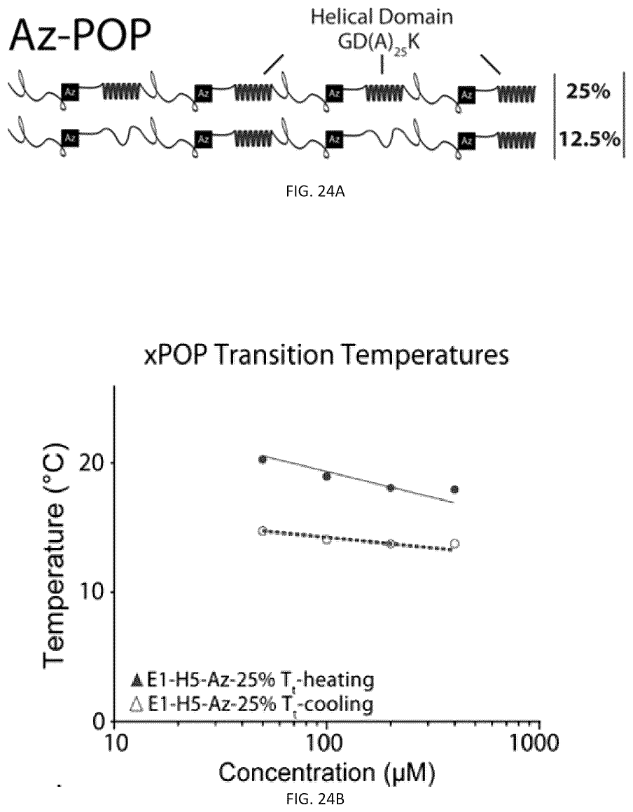

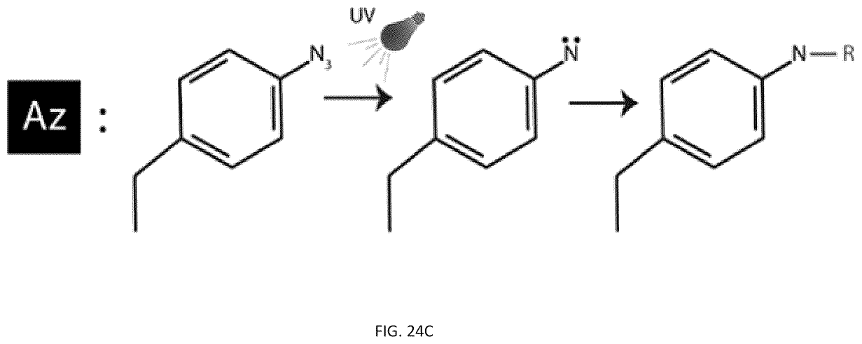

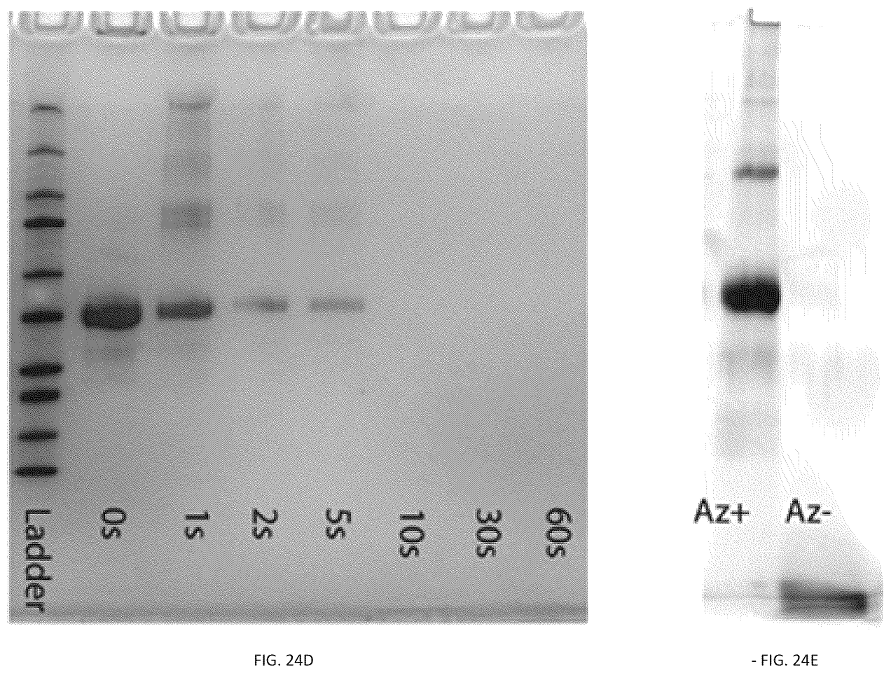

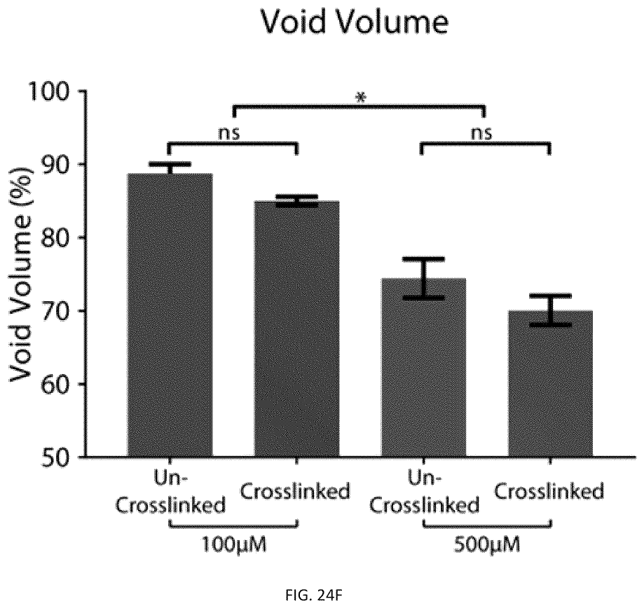

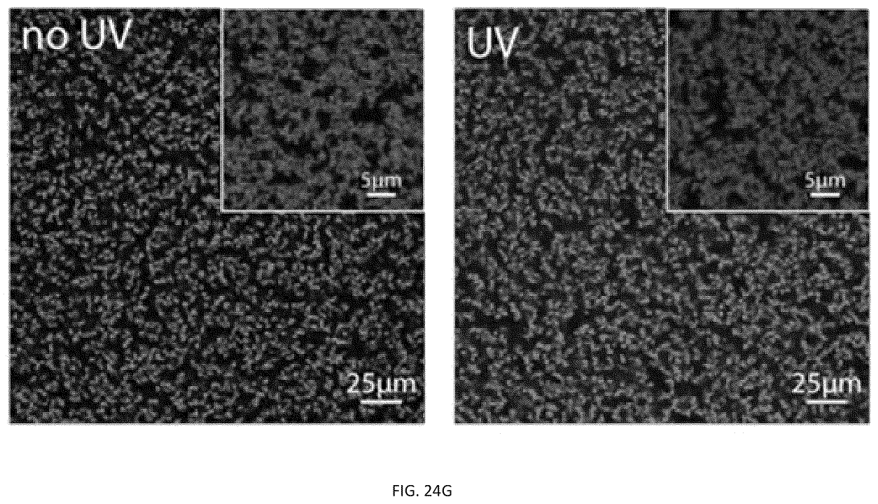

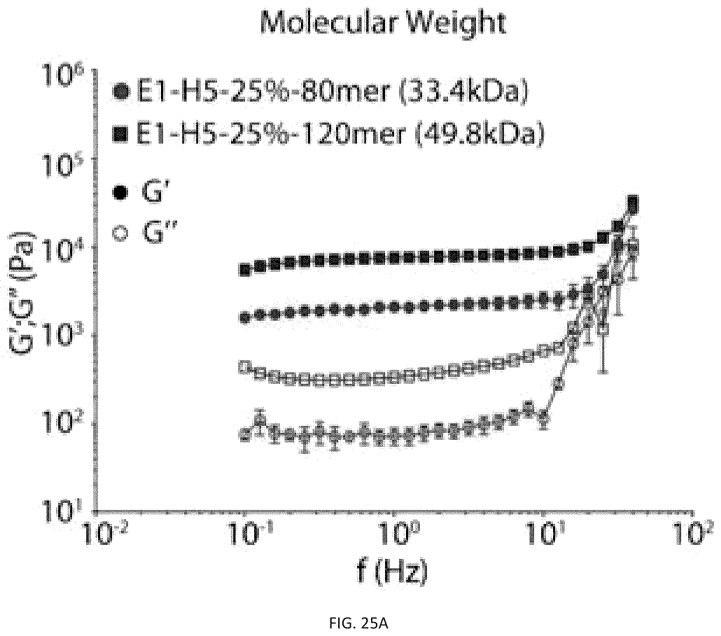

Gene fragments were cloned into a modified pet-24 vector via recursive directional ligation by plasmid reconstruction into chemically competent E. coli. Following their complete synthesis, genes were isolated via restriction digest, and the appropriate fragment cloned into another modified pet-24 vector with a pTac promoter and rrnB terminator instead of the T7 promoter and terminator of the original vector. The plasmids were then con-transfected into c321.ΔA E. coli alongside a pEvol tRNA/aaRS vector with two copies of pAcFRS.1.t1 synthetase—the c321.ΔA genome has previously been edited to remove all instances of the amber stop codon, and the tRNA/aaRS pair has been optimized to recognize the amber stop codon and attach to para-azidophenylalanine (N. W. Choi, J. Kim, S. C. Chapin, T. Duong, E. Donohue, P. Pandey, W. Broom, W. A. Hill, P. S. Doyle, Anal Chem 2012, 84, 9370, which is incorporated by reference herein in its entirety). Liquid cell cultures from 25% glycerol stocks were grown overnight (˜16 hr) in 25 mL 2×YT starter cultures containing 45 μg/mL kanamycin and 25 μg/mL chloramphenicol at 37° C. and 200 rpm. Starter cultures were then transferred to 1 L 2×YT cultures the following morning and grown for −8 hr in the presence of 45 μg/mL kanamycin, 25 μg/mL chloramphenicol, 0.2% arabinose, and 1 mM pAzF at 34° C. and 200 rpm. 1 mM IPTG was then added to induce xPOP expression, and cultures were grown for an additional −16 hr overnight. Proteins were purified using inverse transition cycling as previously described for non-crosslinkable POPs. Purity was determined via SDS-PAGE gel electrophoresis. Samples were then lyophilized and stored at −20° C. All protocols were completed under low-light conditions to avoid undesirable pAzF crosslinking during synthesis and purification. xPOP transition behavior was characterized using a Cary 100 UV-Vis spectrophotometer monitoring optical density at 650 nm. Samples in 1×PBS were heated and cooled at 1° C./min and the point at which the first derivative of the curves was found to be largest in magnitude were recorded as the heating and cooling Tts. Characterization of the UV crosslinkable POPs can be seen in FIG. 24.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A partially ordered polypeptide (POP) comprising: a plurality of disordered domains; and a plurality of structured domains, wherein the POP exhibits phase transition behavior.

Clause 2. The polypeptide of clause 1, wherein the disordered domain comprises at least one of: (i) an amino acid sequence of [VPGXG]ₘ (SEQ ID NO:1), wherein X is any amino acid except proline and m is an integer greater than or equal to 1; (ii) a PG motif comprising an amino acid sequence selected from PG, P(X)ₙG (SEQ ID NO:2), and (B)ₘP(X)ₙG(Z) p (SEQ ID NO:3), or a combination thereof, wherein m, n, and p are independently an integer from 1 to 15, and wherein U, X, and Z are independently any amino acid; (iii) a non-repetitive polypeptide comprising a sequence of at least 60 amino acids, wherein at least about 10% of the amino acids are proline (P), and wherein at least about 20% of the amino acids are glycine (G); (iv) a non-repetitive polypeptide comprising a sequence of at least 60 amino acids, wherein at least about 40% of the amino acids are selected from the group consisting of valine (V), alanine (A), leucine (L), lysine (K), threonine (T), isoleucine (I), tyrosine (Y), serine (S), and phenylalanine (F); and (v) a non-repetitive polypeptide comprising a sequence of at least 60 amino acids, wherein the sequence does not contain three contiguous identical amino acids, wherein any 5-10 amino acid subsequence does not occur more than once in the non-repetitive polypeptide, and wherein when the non-repetitive polypeptide comprises a subsequence starting and ending with proline (P), the subsequence further comprises at least one glycine (G).

Clause 3. The polypeptide of clause 2, wherein the disordered domain comprises an amino acid sequence of [VPGXG]ₘ (SEQ ID NO:1), wherein X is Val, or Ala, or mixture of Ala and Val, and wherein m is an integer from 1 to 50.

Clause 4. The polypeptide of clause 3, wherein X is a mixture of Ala and Val in a ratio from 10:1 to 1:10 (Ala:Val).

Clause 5. The polypeptide of clause 4, wherein X is a mixture of Ala and Val in a ratio of 1:1 or 1:4.

Clause 6. The polypeptide of any one of clauses 1-5, wherein the structured domain comprises at least one of: (i) a polyproline domain, each polyproline domain comprising at least 5 proline residues and having at least about 50% of the amino acids in a PPI polyproline helical conformation or a PPII polyproline helical conformation; and (ii) a polyalanine domain, each polyalanine domain comprising at least 5 alanine residues and having at least about 50% of the amino acids in an alpha-helical conformation.

Clause 7. The polypeptide of clause 6, wherein the structured domain comprises a polyalanine domain.

Clause 8. The polypeptide of clause 7, wherein at least about 60% of the amino acids in each polyalanine domain are in an alpha-helical conformation.

Clause 9. The polypeptide of clause 7 or 8, wherein the polyalanine domain comprises an amino acid sequence of [Bₚ(A)_{q}Zᵣ]ₙ (SEQ ID NO:4) or [(BAₛ)ₜZᵣ]ₙ (SEQ ID NO:5), wherein B is Lys, Arg, Asp, or Glu; A is Ala; Z is Lys, Arg, Asp, or Glu; n is an integer from 1 to 50; p is an integer from 0 to 2; q is an integer from 1 to 50; r is an integer from 0 to 2; s is an integer from 1 to 5; and t is an integer from 1 to 50.

Clause 10. The polypeptide of any one of clauses 1-5 and 7-9, wherein the structured domain comprises (A)₂₅ (SEQ ID NO:6), K(A)₂₅K (SEQ ID NO:7), (KAAAA)₅K (SEQ ID NO:8), or D(A)₂₅K (SEQ ID NO:9), or a combination thereof.

Clause 11. The polypeptide of any one of the preceding clauses, wherein the POP comprises alternating disordered domains and structured domains.

Clause 12. The polypeptide of any one of the preceding clauses, wherein about 4% to about 75% of the POP comprises structured domains.

Clause 13. The polypeptide of any one of the preceding clauses, wherein the POP is soluble below a lower critical solution temperature (LCST).

Clause 14. The polypeptide of any one of the preceding clauses, wherein the POP has a transition temperature of heating (Tt-heating) and a transition temperature of cooling (Tt-cooling).

Clause 15. The polypeptide of clause 14, wherein the transition temperature of heating (Tt-heating) and transition temperature of cooling (Tt-cooling) are identical.

Clause 16. The polypeptide of clause 14, wherein the transition temperature of heating (Tt-heating) is greater than the transition temperature of cooling (Tt-cooling).

Clause 17. The polypeptide of any one of clauses 14-16, wherein the transition temperature of heating (Tt-heating) is concentration-dependent.

Clause 18. The polypeptide of any one of clauses 14-16, wherein the transition temperature of cooling (Tt-cooling) is concentration-independent.

Clause 19. The polypeptide of any one of clauses 14-16, wherein the Tt-heating is primarily determined by the disordered domains, and wherein the Tt-cooling is primarily determined by the structured domains.

Clause 20. The polypeptide of any one of clauses 14-18, wherein the POP forms an aggregate above the Tt-heating.

Clause 21. The polypeptide of clause 20, wherein the aggregate resolubilizes when cooled to below the Tt-cooling.

Clause 22. The polypeptide of clause 20, wherein the aggregate is a stable three-dimensional matrix.

Clause 23. The polypeptide of clause 20, wherein the aggregate is fractal-like.

Clause 24. The polypeptide of clause 20, wherein the aggregate is porous with a void volume.

Clause 25. The polypeptide of clause 24, wherein the void volume is tunable.

Clause 26. A scaffold comprising a plurality of the polypeptide of any one of clauses 1-25 at a temperature greater than the transition temperature, such that the polypeptide forms an aggregate.

Clause 27. A cellular scaffold comprising the scaffold of clause 26, and a plurality of cells.

Clause 28. A method for forming a cellular scaffold, the method comprising: mixing cells with a plurality of the polypeptide of any one of clauses 1-25 at a first temperature less than the transition temperature of the polypeptide, such that the polypeptide does not form an aggregate; and incubating the polypeptides at a second temperature suitable for cellular growth and greater than the transition temperature, such that the polypeptides form an aggregate with the cells encapsulated within, to form the cellular scaffold.

Clause 29. The method of clause 28, further comprising implanting the cellular scaffold into a subject.

Clause 30. A method for forming a cellular scaffold, the method comprising: mixing cells with a plurality of the polypeptide of any one of clauses 1-25 to form a mixture, at a first temperature less than the transition temperature of the polypeptide, such that the polypeptide does not form an aggregate; and injecting the mixture at the first temperature into a subject, wherein the subject is at a second temperature greater than the transition temperature, such that the polypeptides form an aggregate with the cells encapsulated within, to form the cellular scaffold in the subject.

Clause 31. A method for forming a scaffold, the method comprising: injecting into a subject a plurality of the polypeptide of any one of clauses 1-23 at a first temperature less than the transition temperature of the polypeptide, such that the polypeptide does not form an aggregate prior to injection, wherein the subject is at a second temperature greater than the transition temperature, such that the polypeptides form an aggregate to form the scaffold in the subject.

Clause 32. The method of any one of clauses 28-30, wherein the cells within the scaffold integrate into the surrounding cells or tissues of the subject.

Clause 33. The method of any one of clauses 28-31, wherein the cells of the subject surrounding the scaffold integrate into the scaffold.

Clause 34. The method of clause 32, wherein the cells within the scaffold modify the surrounding cells or tissues of the subject.

Clause 35. The method of any one of clauses 29-34, wherein the cells within the scaffold, the cells integrating into the scaffold, or the cells modified by the scaffold form new vasculature.

Clause 36. The method of clause 28, further comprising: reducing the temperature to the first temperature, such that the aggregate/scaffold solubilizes; and separating the cells from the solubilized scaffold.

Clause 37. The method of clause 36, wherein the separating step comprises centrifugation.

Clause 38. The scaffold or method of any one of clauses 26-30 and 32-37, wherein the cells comprise stem cells, bacterial cells, or human tissue cells.

Clause 39. The scaffold or method of any one of clauses 26-38, wherein the scaffold has low immunogenicity or low antigenicity.

Clause 40. The scaffold or method of any one of clauses 26-39, wherein the scaffold promotes at least one of cell growth, recruitment, and differentiation.

Clause 41. A drug delivery composition comprising: a plurality of POPs according to any one of clauses 1-25, self-assembled into an aggregate above the Tₜ-heating; and an agent encapsulated within the aggregate.

Clause 42. The drug delivery composition of clause 41, wherein the agent recruits dendritic cells.

Clause 43. A method of delivering an agent to a subject, the method comprising: encapsulating the agent in an aggregate, the aggregate comprising a plurality of POPs according to any one of clauses 1-25; and administering the aggregate to the subject.

Clause 44. A method of treating a disease in a subject in need thereof, the method comprising administering the drug delivery composition of clause 41 to the subject.

Clause 45. The method of clause 44, wherein administering the drug delivery composition results in the formation of new vasculature, wound healing, or a combination thereof in the subject.

Clause 46. A method of increasing the maximum tolerated dose of an agent, the method comprising: encapsulating the agent in an aggregate of POPs according to any one of clauses 1-25; and administering the agent-encapsulated aggregate to a subject.

Clause 47. The composition of clause 41 or the method of any one of clauses 43-46, wherein the agent comprises a small molecule, a polynucleotide, a polypeptide, a carbohydrate, or a combination thereof.

Sequences
(SEQ ID NO: 1)
[VPGXG]ₘ
(SEQ ID NO: 2)
P(X)ₙG
(SEQ ID NO: 3)
(B)ₘP(X)ₙG(Z)ₚ
(SEQ ID NO: 4)
[Bp(A)_{q}Zr]ₙ
(SEQ ID NO: 5)
[(BAₛ)ₜZr]ₙ
(SEQ ID NO: 6)
(A)₂₅
(SEQ ID NO: 7)
K(A)₂₅K
(SEQ ID NO: 8)
(KAAAA)₅K
(SEQ ID NO: 9)
D(A)₂₅K
(SEQ ID NO: 10)
PXXG
(SEQ ID NO: 11)
PXXXG
(SEQ ID NO: 12)
PXXXXG
(SEQ ID NO: 13)
PXXXXXG
(SEQ ID NO: 14)
PXXXXXXG
(SEQ ID NO: 15)
PXXXXXXXG
(SEQ ID NO: 16)
PXXXXXXXXG
(SEQ ID NO: 17)
PXXXXXXXXXG
(SEQ ID NO: 18)
PXXXXXXXXXXG
(SEQ ID NO: 19)
PXXXXXXXXXXXG
(SEQ ID NO: 20)
PXXXXXXXXXXXXG
(SEQ ID NO: 21)
PXXXXXXXXXXXXXG
(SEQ ID NO: 22)
PXXXXXXXXXXXXXXG
(SEQ ID NO: 23)
PXXXXXXXXXXXXXXXG
(SEQ ID NO: 24)
TGTGGGTGTTCCGGGCGTAGGTGTCCCAGGTGTGGGCGTA
CCGGGCGTTGGTGTTCCTGGTGTCGGCGTGCCGGG
(SEQ ID NO: 25)
CGGCACGCCGACACCAGGAACACCAACGCCCGGTACGCCC
ACACCTGGGACACCTACGCCCGGAACACCCACACC
(SEQ ID NO: 26)
CGTGGGTGTTCCGGGCGTAGGTGTCCCAGGTGCGGGCGTA
CCGGGCGTTGGTGTTCCTGGTGTCGGCGTGCCGGG
(SEQ ID NO: 27)
CGGCACGCCGACACCAGGAACACCAACGCCCGGTACGCCC
GCACCTGGGACACCTACGCCCGGAACACCCACGCC
(SEQ ID NO: 28)
CGCCGGAGTGCCAGGCGTGGGTGTTCCAGGAGCAGGCGTT
CCAGGTGTGGGTGTTCCTGG
(SEQ ID NO: 29)
AGGAACACCCACACCTGGAACGCCTGCTCCTGGAACACCC
ACGCCTGGCACTCCGGCGCC
(SEQ ID NO: 30)
TGCGGCCGCAGCTGCGGCGGCAGCCGCGGCTGCCGCGGCT
GCAGCGGCAGCCGCGGCTGCGGCGGCCGCAGCTGCGGG
(SEQ ID NO: 31)
CGCAGCTGCGGCCGCCGCAGCCGCGGCTGCCGCTGCAGCC
GCGGCAGCCGCGGCTGCCGCCGCAGCTGCGGCCGCACC
(SEQ ID NO: 32)
TAAAGCGGCCGCAGCTGCGGCGGCAGCCGCGGCTGCCGCG
GCTGCAGCGGCAGCCGCGGCTGCGGCGGCCGCAGCTGCGA
AAGG
(SEQ ID NO: 33)
TTTCGCAGCTGCGGCCGCCGCAGCCGCGGCTGCCGCTGCA
GCCGCGGCAGCCGCGGCTGCCGCCGCAGCTGCGGCCGCTT
TACC
(SEQ ID NO: 34)
TAAAGCGGCCGCAGCTAAAGCCGCGGCAGCGAAAGCAGCC
GCGGCGAAAGCCGCAGCTGCGAAAGCGGCAGCCGCGAAGG
G
(SEQ ID NO: 35)
CTTCGCGGCTGCCGCTTTCGCAGCTGCGGCTTTCGCCGCG
GCTGCTTTCGCTGCCGCGGCTTTAGCTGCGGCCGCTTTAC
C
(SEQ ID NO: 36)
TGATGCGGCCGCAGCTGCGGCGGCAGCCGCGGCTGCCGCG
GCTGCAGCGGCAGCCGCGGCTGCGGCGGCCGCAGCTGCGA
AAGG
(SEQ ID NO: 37)
TTTCGCAGCTGCGGCCGCCGCAGCCGCGGCTGCCGCTGCA
GCCGCGGCAGCCGCGGCTGCCGCCGCAGCTGCGGCCGCAT
CACC
(SEQ ID NO: 38)
AAAAA
(SEQ ID NO: 39)
VPGVG

1. A partially ordered polypeptide (POP) comprising:
a plurality of disordered domains; and
a plurality of structured domains, wherein the POP exhibits phase transition behavior.

2. The polypeptide of claim 1, wherein the disordered domain comprises at least one of:
(i) an amino acid sequence of [VPGXG]ₘ (SEQ ID NO:1), wherein X is any amino acid except proline and m is an integer greater than or equal to 1;
(ii) a PG motif comprising an amino acid sequence selected from PG, P(X)ₙG (SEQ ID NO:2), and (B)ₘP(X)ₙG(Z)ₚ (SEQ ID NO:3), or a combination thereof, wherein m, n, and p are independently an integer from 1 to 15, and wherein U, X, and Z are independently any amino acid;
(iii) a non-repetitive polypeptide comprising a sequence of at least 60 amino acids,
wherein at least about 10% of the amino acids are proline (P), and
wherein at least about 20% of the amino acids are glycine (G);
(iv) a non-repetitive polypeptide comprising a sequence of at least 60 amino acids,
wherein at least about 40% of the amino acids are selected from the group consisting of valine (V), alanine (A), leucine (L), lysine (K), threonine (T), isoleucine (I), tyrosine (Y), serine (S), and phenylalanine (F); and
(v) a non-repetitive polypeptide comprising a sequence of at least 60 amino acids,
wherein the sequence does not contain three contiguous identical amino acids,
wherein any 5-10 amino acid subsequence does not occur more than once in the non-repetitive polypeptide, and
wherein when the non-repetitive polypeptide comprises a subsequence starting and ending with proline (P), the subsequence further comprises at least one glycine (G).

3. The polypeptide of claim 2, wherein the disordered domain comprises an amino acid sequence of [VPGXG]ₘ (SEQ ID NO:1), wherein X is Val, or Ala, or mixture of Ala and Val, and wherein m is an integer from 1 to 50.

4. The polypeptide of claim 3, wherein X is a mixture of Ala and Val in a ratio from 10:1 to 1:10 (Ala:Val).

5. The polypeptide of claim 4, wherein X is a mixture of Ala and Val in a ratio of 1:1 or 1:4.

6. The polypeptide of any one of claims 1-5, wherein the structured domain comprises at least one of:
(i) a polyproline domain, each polyproline domain comprising at least 5 proline residues and having at least about 50% of the amino acids in a PPI polyproline helical conformation or a PPII polyproline helical conformation; and
(ii) a polyalanine domain, each polyalanine domain comprising at least 5 alanine residues and having at least about 50% of the amino acids in an alpha-helical conformation.

7. The polypeptide of claim 6, wherein the structured domain comprises a polyalanine domain.

8. The polypeptide of claim 7, wherein at least about 60% of the amino acids in each polyalanine domain are in an alpha-helical conformation.

9. The polypeptide of claim 7 or 8, wherein the polyalanine domain comprises an amino acid sequence of [Bₚ(A)_{q}Zᵣ]ₙ (SEQ ID NO:4) or [(BAₛ)ₜZᵣ]ₙ (SEQ ID NO:5),
wherein B is Lys, Arg, Asp, or Glu;
A is Ala;
Z is Lys, Arg, Asp, or Glu;
n is an integer from 1 to 50;
p is an integer from 0 to 2;
q is an integer from 1 to 50;
r is an integer from 0 to 2;
s is an integer from 1 to 5; and
t is an integer from 1 to 50.

10. The polypeptide of any one of claims 1-5 and 7-9, wherein the structured domain comprises (A)₂₅ (SEQ ID NO:6), K(A)₂₅K (SEQ ID NO:7), (KAAAA)₅K (SEQ ID NO:8), or D(A)₂₅K (SEQ ID NO:9), or a combination thereof.

11. The polypeptide of any one of the preceding claims, wherein the POP comprises alternating disordered domains and structured domains.

12. The polypeptide of any one of the preceding claims, wherein about 4% to about 75% of the POP comprises structured domains.

13. The polypeptide of any one of the preceding claims, wherein the POP is soluble below a lower critical solution temperature (LCST).

14. The polypeptide of any one of the preceding claims, wherein the POP has a transition temperature of heating (Tt-heating) and a transition temperature of cooling (Tt-cooling).

15. The polypeptide of claim 14, wherein the transition temperature of heating (Tt-heating) and transition temperature of cooling (Tt-cooling) are identical.

16. The polypeptide of claim 14, wherein the transition temperature of heating (Tt-heating) is greater than the transition temperature of cooling (Tt-cooling).

17. The polypeptide of any one of claims 14-16, wherein the transition temperature of heating (Tt-heating) is concentration-dependent.

18. The polypeptide of any one of claims 14-16, wherein the transition temperature of cooling (Tt-cooling) is concentration-independent.

19. The polypeptide of any one of claims 14-16, wherein the Tt-heating is primarily determined by the disordered domains, and wherein the Tt-cooling is primarily determined by the structured domains.

20. The polypeptide of any one of claims 14-18, wherein the POP forms an aggregate above the Tt-heating.

21. The polypeptide of claim 20, wherein the aggregate resolubilizes when cooled to below the Tt-cooling.

22. The polypeptide of claim 20, wherein the aggregate is a stable three-dimensional matrix.

23. The polypeptide of claim 20, wherein the aggregate is fractal-like.

24. The polypeptide of claim 20, wherein the aggregate is porous with a void volume.

25. The polypeptide of claim 24, wherein the void volume is tunable.

26. A scaffold comprising a plurality of the polypeptide of any one of claims 1-25 at a temperature greater than the transition temperature, such that the polypeptide forms an aggregate.

27. A cellular scaffold comprising the scaffold of claim 26, and a plurality of cells.

28. A method for forming a cellular scaffold, the method comprising:
mixing cells with a plurality of the polypeptide of any one of claims 1-25 at a first temperature less than the transition temperature of the polypeptide, such that the polypeptide does not form an aggregate; and
incubating the polypeptides at a second temperature suitable for cellular growth and greater than the transition temperature, such that the polypeptides form an aggregate with the cells encapsulated within, to form the cellular scaffold.

29. The method of claim 28, further comprising implanting the cellular scaffold into a subject.

30. A method for forming a cellular scaffold, the method comprising:
mixing cells with a plurality of the polypeptide of any one of claims 1-25 to form a mixture, at a first temperature less than the transition temperature of the polypeptide, such that the polypeptide does not form an aggregate; and
injecting the mixture at the first temperature into a subject, wherein the subject is at a second temperature greater than the transition temperature, such that the polypeptides form an aggregate with the cells encapsulated within, to form the cellular scaffold in the subject.

31. A method for forming a scaffold, the method comprising:
injecting into a subject a plurality of the polypeptide of any one of claims 1-23 at a first temperature less than the transition temperature of the polypeptide, such that the polypeptide does not form an aggregate prior to injection,
wherein the subject is at a second temperature greater than the transition temperature, such that the polypeptides form an aggregate to form the scaffold in the subject.

32. The method of any one of claims 28-30, wherein the cells within the scaffold integrate into the surrounding cells or tissues of the subject.

33. The method of any one of claims 28-31, wherein the cells of the subject surrounding the scaffold integrate into the scaffold.

34. The method of claim 32, wherein the cells within the scaffold modify the surrounding cells or tissues of the subject.

35. The method of any one of claims 29-34, wherein the cells within the scaffold, the cells integrating into the scaffold, or the cells modified by the scaffold form new vasculature.

36. The method of claim 28, further comprising:
reducing the temperature to the first temperature, such that the aggregate/scaffold solubilizes; and
separating the cells from the solubilized scaffold.

37. The method of claim 36, wherein the separating step comprises centrifugation.

38. The scaffold or method of any one of claims 26-30 and 32-37, wherein the cells comprise stem cells, bacterial cells, or human tissue cells.

39. The scaffold or method of any one of claims 26-38, wherein the scaffold has low immunogenicity or low antigenicity.

40. The scaffold or method of any one of claims 26-39, wherein the scaffold promotes at least one of cell growth, recruitment, and differentiation.

41. A drug delivery composition comprising:
a plurality of POPs according to any one of claims 1-25, self-assembled into an aggregate above the Tt-heating; and
an agent encapsulated within the aggregate.

42. The drug delivery composition of claim 41, wherein the agent recruits dendritic cells.

43. A method of delivering an agent to a subject, the method comprising:
encapsulating the agent in an aggregate, the aggregate comprising a plurality of POPs according to any one of claims 1-25; and
administering the aggregate to the subject.

44. A method of treating a disease in a subject in need thereof, the method comprising administering the drug delivery composition of claim 41 to the subject.

45. The method of claim 44, wherein administering the drug delivery composition results in the formation of new vasculature, wound healing, or a combination thereof in the subject.

46. A method of increasing the maximum tolerated dose of an agent, the method comprising:
encapsulating the agent in an aggregate of POPs according to any one of claims 1-25; and
administering the agent-encapsulated aggregate to a subject.

47. The composition of claim 41 or the method of any one of claims 43-46, wherein the agent comprises a small molecule, a polynucleotide, a polypeptide, a carbohydrate, or a combination thereof.


SEQUENCE LISTING (39 sequences)

SEQ ID NO: 1 - protein, 5 aa, synthetic construct
  REPEAT 1..5: note = sequence 1-5 may repeat one or more times
  VARIANT 4: note = X is any amino acid except proline
VPGXG

SEQ ID NO: 2 - skipped

SEQ ID NO: 3 - protein, 47 aa, synthetic construct
  VARIANT 2..15: note = Asx at positions 2-15 can either be present or absent
  VARIANT 17..31: note = X is any amino acid independent of Glx at positions 32-47
  VARIANT 32..47: note = Glx at positions 32-47 are independent of X at positions 17-31
  VARIANT 17..31: note = where any one or all amino acids at positions 18-31 can be either present or absent
BBBBBBBBBBBBBBBPXXXXXXXXXXXXXXXGZZZZZZZZZZZZZZZ

SEQ ID NO: 4 - protein, 54 aa, synthetic construct
  VARIANT 1..2: note = Any or all Asx at positions 1-2 can be either present or absent
  REPEAT 1..54: note = sequence 1-54 may repeat 1-50 times
  VARIANT 4..52: note = Any one or all Ala at positions 4-52 can be either present or absent
  VARIANT 53..54: note = Any or all Glx at positions 53-54 can be either present or absent
BBAAAAAAAAAAAAAAAAAAAAAAAAAAAAAAAAAAAAAAAAAAAAAAAAAAZZ

SEQ ID NO: 5 - skipped

SEQ ID NO: 6 - protein, 25 aa, synthetic construct
AAAAAAAAAAAAAAAAAAAAAAAAA

SEQ ID NO: 7 - protein, 27 aa, synthetic construct
KAAAAAAAAAAAAAAAAAAAAAAAAAK

SEQ ID NO: 8 - protein, 26 aa, synthetic construct
KAAAAKAAAAKAAAAKAAAAKAAAAK

SEQ ID NO: 9 - protein, 27 aa, synthetic construct
DAAAAAAAAAAAAAAAAAAAAAAAAAK

SEQ ID NO: 10-23 - skipped (14 entries)

SEQ ID NO: 24 - DNA, 75 bp, synthetic construct
  source 1..75: mol_type = other DNA
tgtgggtgttccgggcgtaggtgtcccaggtgtgggcgtaccgggcgttggtgttcctggtgtcggcgtgccggg

SEQ ID NO: 25 - DNA, 75 bp, synthetic construct
  source 1..75: mol_type = other DNA
cggcacgccgacaccaggaacaccaacgcccggtacgcccacacctgggacacctacgcccggaacacccacacc

SEQ ID NO: 26 - DNA, 75 bp, synthetic construct
  source 1..75: mol_type = other DNA
cgtgggtgttccgggcgtaggtgtcccaggtgcgggcgtaccgggcgttggtgttcctggtgtcggcgtgccggg

SEQ ID NO: 27 - DNA, 75 bp, synthetic construct
  source 1..75: mol_type = other DNA
cggcacgccgacaccaggaacaccaacgcccggtacgcccgcacctgggacacctacgcccggaacacccacgcc

SEQ ID NO: 28 - DNA, 60 bp, synthetic construct
  source 1..60: mol_type = other DNA
cgccggagtgccaggcgtgggtgttccaggagcaggcgttccaggtgtgggtgttcctgg

SEQ ID NO: 29 - DNA, 60 bp, synthetic construct
  source 1..60: mol_type = other DNA
aggaacacccacacctggaacgcctgctcctggaacacccacgcctggcactccggcgcc

SEQ ID NO: 30 - DNA, 78 bp, synthetic construct
  source 1..78: mol_type = other DNA
tgcggccgcagctgcggcggcagccgcggctgccgcggctgcagcggcagccgcggctgcggcggccgcagctgcggg

SEQ ID NO: 31 - DNA, 78 bp, synthetic construct
  source 1..78: mol_type = other DNA
cgcagctgcggccgccgcagccgcggctgccgctgcagccgcggcagccgcggctgccgccgcagctgcggccgcacc

SEQ ID NO: 32 - DNA, 84 bp, synthetic construct
  source 1..84: mol_type = other DNA
taaagcggccgcagctgcggcggcagccgcggctgccgcggctgcagcggcagccgcggctgcggcggccgcagctgcgaaagg

SEQ ID NO: 33 - DNA, 84 bp, synthetic construct
  source 1..84: mol_type = other DNA
tttcgcagctgcggccgccgcagccgcggctgccgctgcagccgcggcagccgcggctgccgccgcagctgcggccgctttacc

SEQ ID NO: 34 - DNA, 81 bp, synthetic construct
  source 1..81: mol_type = other DNA
taaagcggccgcagctaaagccgcggcagcgaaagcagccgcggcgaaagccgcagctgcgaaagcggcagccgcgaaggg

SEQ ID NO: 35 - DNA, 81 bp, synthetic construct
  source 1..81: mol_type = other DNA
cttcgcggctgccgctttcgcagctgcggctttcgccgcggctgctttcgctgccgcggctttagctgcggccgctttacc

SEQ ID NO: 36 - DNA, 84 bp, synthetic construct
  source 1..84: mol_type = other DNA
tgatgcggccgcagctgcggcggcagccgcggctgccgcggctgcagcggcagccgcggctgcggcggccgcagctgcgaaagg

SEQ ID NO: 37 - DNA, 84 bp, synthetic construct
  source 1..84: mol_type = other DNA
tttcgcagctgcggccgccgcagccgcggctgccgctgcagccgcggcagccgcggctgccgccgcagctgcggccgcatcacc

SEQ ID NO: 38 - protein, 5 aa, synthetic construct
AAAAA

SEQ ID NO: 39 - protein, 5 aa, synthetic construct
VPGVG